(19) United States
(12) Patent Application Publication
(10) Pub. No.: US 2023/0277449 A1
(43) Pub. Date: Sep. 7, 2023

(54) ORAL CAVITY POLYMERIC DELIVERY SYSTEMS

(71) Applicant: YISSUM RESEARCH DEVELOPMENT COMPANY OF THE HEBREW UNIVERSITY OF JERUSALEM LTD., Jerusalem (IL)

(72) Inventors: Abraham Jacob DOMB, Jerusalem (IL); Amnon HOFFMAN, Jerusalem (IL); Constantin ITIN, Beit Shemesh (IL)

(21) Appl. No.: 18/005,197

(22) PCT Filed: Jul. 14, 2021
(86) PCT No.: PCT/IL2021/050860

§ 371 Date: Jan. 12, 2023

Related U.S. Application Data
(60) Provisional application No. 62/705,774, filed on Jul. 15, 2020.
(60) Provisional application No. 62/705,775, filed on Jul. 15, 2020.

(51) Int. Cl. A61K 9/00; A61K 9/20; A61K 45/06

(52) U.S. Cl. CPC A61K 9/006; A61K 9/2018; A61K 9/2027; A61K 9/2054; A61K 45/06

(57) ABSTRACT

The invention provides devices, formulations, and methods for systemic delivery of agents in the oral and buccal cavity and the gastrointestinal system, including immediate and prolonged drug delivery. The invention is particularly applicable to poorly water-soluble agents and drugs.

### TECHNOLOGICAL FIELD

The invention generally pertains to devices, formulations, and methods for local and systemic delivery of actives and drugs through mucosal tissues and orans of the oral cavity and esophagus, including immediate and prolonged delivery. The invention is particularly applicable to poorly water-soluble agents and drugs.

### BACKGROUND

Among various routes of drug delivery, the oral route is probably the most preferable by patients and practitioners alike. Oral administration, however, has its disadvantages. One of the more notorious is the hepatic first-pass metabolism and enzymatic degradation inside the gastrointestinal (GI) tract. As a result, alternative methods of drug absorption are being developed, with specific emphasis on absorptive mucosae or transmucosal routes, i.e., administration through the membrane linings of ocular, rectal, vaginal, nasal or oral cavities. Transmucosal routes provide a number of benefits for systemic drug delivery over oral delivery such as a bypass of the first-pass effect, avoidance of pre-systemic elimination inside the GI tract, and, for some drugs, preservation of bacterial flora and enzymatic activity contributing to drug absorption.

The nasal cavity as an advantageous site for systemic drug delivery has already reached certain commercial status with drugs such as thyrocalcitonin [1-2] and LHRH [3]. It suffers however from several weakness, especially in chronic applications, such as risks of irritation and irreversible damage to the ciliary function in the nasal cavity, owing to which this route remains less preferable. The same is true for the other transmucosal routes, where poor patient adherence characteristic of rectal, vaginal and ocular deliveries renders them limited to specific applications and mainly to local treatments.

The situation is different for transmucosal delivery through the oral cavity. As mentioned above, the oral transmucosal drug delivery bypasses the first-pass effect (or metabolism) and avoids pre-systemic elimination in the GI tract. The oral membrane is comparatively permeable and furnished with an extensive blood supply. It is sturdy and shows short recovery after stress or injury [4]. All these make the oral mucosa an engaging and potentially preferred site for systemic drug delivery.

Transmucosal delivery of drugs through the oral cavity is generally classified into three categories depending on the site of administration, all three involve local and systemic drug delivery: (a) sublingual delivery via mucosal membranes lining the ground of the mouth; (b) buccal delivery via the mucosal membranes lining the buccal mucosa or the cheeks; and (c) and delivery via the oral cavity membranes.

Buccal mucosa has several properties making it an advantageous site for systemic drug delivery using various types of removable devices in the form of tablets or films. These include being approachable for administration and removal of a device in case of adverse effect, large enough area (~50 cm²) available for permeation, relative stiffness enabling adherence of a device, and cellular turnover time of 4-14 days enabling to maintain the adherence for many hours or even days while retaining the ability for rapid recovery in case of tissue damage. In addition, the microenvironment at the site of attachment can be directly and easily modified enabling control over drug release. Substances successfully permeating the buccal mucosa enter the systemic circulation without being exposed to the harsh conditions of the GI tract, barriers of absorption after per-oral administration and first-pass metabolism in the enterocytes and liver [5].

### REFERENCES

Dal Negra, R., Turco, P., Pomari, C., and Trevisan, F., Calcitonin nasal spray in patienmts with chronic asthma: a double-blind crossover study vs placebo, Int. J. Clin. Pharmacol. Ther. Toxicol., 29:144-146, 1991.

Plosker, G.L. and McTavish, D., Intranasal salcatonin (salmon calcitonin). A review of its pharmacological properties and role in the management of postmenopausal osteoporosis, Drugs Aging, 8:378-400, 1996.

Nakane, S., Kakumoto, M., Yulimatsu, K., and Chien, Y.W., Oramucosal delivery of LHRH: Pharmacokinetic studies of controlled and enhanced transmucosal permeation, Pharm. Dev. Tech., 1:251-259, 1996.

Squier, C.A., The permeability of oral mucosa, Crit. Rev. Oral Biol. Med., 2:13-32, 1991.

E. Meng-Lund, E. Marxen, A.M.L. Pedersen, A. Müllertz, B. Hyrup, R. Holm, J. Jacobsen, Ex vivo correlation of the permeability of metoprolol across human and porcine buccal mucosa, J Pharm Sci, 103:2053-2061, 2014.

### GENERAL DESCRIPTION

Oral mucosal drug delivery is an alternative method of systemic drug delivery that offers several advantages over both injectable and enteral methods, and further enhances drug bioavailability because the mucosal surfaces are usually rich in blood supply, providing the means for rapid drug transport to the systemic circulation and avoiding, in most cases, degradation by first-pass hepatic metabolism. The systems contact with the absorption surface resulting in a better absorption, and also prolong residence time at the site of application to permit once or twice daily dosing. For some drugs, this results in rapid onset of action via a more comfortable and convenient delivery route than the intravenous (IV) route. Not all drugs, however, can be administered through the oral mucosa because of the characteristics of the oral mucosa and the physicochemical properties of the drug. Although many drugs have been evaluated for oral transmucosal delivery, few are commercially available owing to high costs associated with developing this type of product. Transmucosal products are a relatively new drug delivery strategy. Transmucosal drug delivery promises four times the absorption rate of skin, for example.

Solubility behavior of a given candidate drug is one of the biggest challenges of successful drug delivery. About 40% of drugs developed by Pharma industry and about 90% of the drugs in development pipeline are poorly soluble drugs. Poorly water-soluble drugs tend to be eliminated from the GI tract before they get the opportunity to be fully dissolve and be absorbed into the blood circulation. This results in low bioavailability and poor dose response proportionality that hinders their clinical translations. Dose augmentation at times causes topical toxicity in the GI tract and results in decline in patient compliance. In general, poorly water-soluble drugs show a number of negative clinical effects including potentially serious issues of inter-patient variability, inefficient treatment, higher patient costs, and more importantly, increased risks of toxicity.

Several technologies have been successfully employed to enhance the solubility, dissolution, and bioavailability of poorly soluble drugs, using particle size reduction, solid dispersions, lipid-based delivery systems, inclusion complexes, and others. Still, there is need for development of new approaches that will successfully overcome the limitations of poorly soluble drugs and also poorly permeable drugs.

The main objective of the present invention is to provide a convenient and versatile device for controlled delivery of a wide range of actives and drugs through the tissues of oral cavity and throat, including the oral mucosa. The devices of the invention can be applied for local and systemic drug delivery for treatment or alleviation of various clinical and pre-clinical conditions, such as halitosis, smoking withdrawal, etc.

One of the advantages of the present technology is in its applicability to poorly water-soluble actives and drugs, and its potential to provide a solution to the problems of poor solubility, bioavailability, and poor absorption. This feature is attributed to the use of a specific pre-formulation wherein the poorly water-soluble active(s) are incorporated and whereby they are atomized and presented into the device in a more absorbable, permeable, and bioavailable form.

Another important advantage resides in its ability to provide immediate and/or prolonged release of actives into the oral tissues and blood circulation, and further to use combinations of actives, and to provide unilateral and bilateral release of actives into the oral tissues and/or oral cavity. Naturally, the range of these abilities stems from a number of elements, e.g., components of the pre-formulation (dispersants, lipids and amphiphilic materials) providing protective and controlled release properties to actives included in the device; chemical components of the device (various poly(carboxylic acids) and small molecule alcohols) providing the features of shape, flexibility and texture and attributing various adhesion, durability or disintegration properties to the device; and the oral mucosa per se contributing to a faster absorption and delivery of actives due to abundant blood supply and permeability.

One important distinction of the present technology is in being solvent-free, i.e., the formulation and the other chemical components of the devices of the invention are provided as solid powders and mixed in various proportions, without any solvents, and then molded into tablets or films of desired shape, thickness, and size, as per the specific applications and dimensions of the human oral cavity. This exceptionally straightforward and simple approach for producing a wide versatility of highly adaptable products is one of the advantages of this invention.

Another distinction of the technology is in being applied and acting through the mucous membrane lining tissues and/or muscle tissues of the oral cavity and throat, and in being adapted to fit the dimensions and architecture of the oral cavity, e.g., the gingivae, hard palate, cheek mucosa, mouth floor and tongue. This feature is attributed to the fast-dissolving and adherence properties of the device owing to which it remains attached and dissolves in situ in the oral cavity to permit therapeutically meaningful release and delivery of actives.

In practical terms, the invention provides hard and flexible films and tablets that can be made from various poly(carboxylic acid) polymers (up to 90%) and edible alcohols, and preformulated and atomized lipophilic or poorly water-soluble actives, which mixed and molded under pressure into desired shapes and forms.

Of particular interest is the feature of controlled adhesiveness of the devices of the invention, which is attributed to and can be modulated by specific features such as texture (rough and smooth tablets), certain additives (sodium carbonate) and others, and further, the use of this feature to provide various types of devices with symmetric and asymmetric adhesive properties.

As the first prototype, the inventors have developed a bio-adhesive devices (tablets or sticker) that can adhere to a tissue surface in the oral cavity (EXAMPLE 1). The adhesive stickers of the invention can be made with various degrees of thickness, flexibility and adherence, and can adhere to the tissue on one side or two sides. The stickers are made from mixtures of polyols and polycarboxylic acids with addition of small molecule alcohols that provide flexibility without affecting the adhesion properties. The stickers further contain preformulated or non-preformulated active agent(s) to be delivered via the oral mucosa and/or the oral cavity. The active can be any pharmacological and non-pharmacological active for maintenance of health or alleviation, treatment or prevention of abnormal conditions or disorders, encompassing oral health and general health and systemic conditions or disorders. Certain examples of such agents are drugs, peptides, proteins, nucleic acids, natural and synthetic drugs and further, herbal remedies, nutrients, and vitamins.

In terms of polymers, it is contemplated that the stickers of the invention can be made of various types of water-soluble or hydrophilic polymers. In many cases, the devices of the invention use polycarboxylic acids or combinations of polyols and polycarboxylic acids. Non-limiting examples are hydroxy propyl cellulose, hydroxypropyl methyl cellulose and their amylose derivatives, polyvinyl alcohol, natural polysaccharide gums, sulfated and non-sulfated carrageenans, hyaluronic acid, chitosan and their mixtures. Another example is the group of crosslinked poly(acrylic acid) and methacrylic acid and their copolymers, alginates, hyaluronic acid, carboxymethyl cellulose and their mixtures.

In terms of edible alcohols, suitable examples are ethanol, isopropanol, glycerol, monoglycerides, ethyl lactate, short chain polyethylene glycols, and tributyl citrate.

In terms of releasable active agents, attractive candidates are analgesics, anesthetics, antiseptics, antibacterial agents, antiviral agents, disinfectants, anti-halitosis agents, anti-inflammatory agents, opioids, melatonin, caffeine, nicotine, stimulating agents, sleep inducing agents and antidepressants, and further complex actives such as herbal and animal extracts, probiotic bacteria or fungi, biological drugs such as peptides, proteins, oligos or polynucleotides, and antibodies.

The active can be presented in the device (sticker) in a formulated atomized form using the pre-formulation of the invention, or alternatively, it can be directly incorporated into the device. While the first is suitable for poorly water-soluble or lipophilic actives, the second can be used for water-soluble and highly permeable actives. From another point on view, the actives can be directed to treating or alleviation of oral disorders, or alternatively, actives intended for systemic delivery and targeting other tissues and sites.

The sticker can be double- or single-sided in terms of attaching properties and laterality of active release. Such sticker can be made of films with different textures on both sides or by layering of films with different adhesive properties and different composition of actives - actives intended for release into the tissue and actives intended for releaase into the oral cavity.

Another type of stickers is site specific stickers that can be placed on lesions or ulcers in the oral cavity (e.g., aphthae).

Another interesting application is gastro-retentive films that remain in the stomach to allow controlled release or sustained release of incorporated drugs in the stomach and the GI track. In many instances, such film is provided in a capsule or a carrier for easy swallowing to be released and unfolded in the stomach because to its size or floating properties. One example of such gastro-retentive delivery systems are films containing L-Dopa, one of the common drugs for treating Parkinson disease (PD).

The second prototype produced within the present framework has been a buccal device for systemic immediate and/or prolonged delivery of actives with poor or intermediate water-solubility. The device is non-invasive and safe, and can be adapted for delivery of actives with complex kinetics or narrow therapeutic window (EXAMPLE 2).

The inventors presently exemplify a buccal mucoadhesive, biocompatible and biodegradable delivery device in a form of adhesive tablet (ABPD) containing a hydrogel-based core comprising apomorphine (APO) - an active with intermediate lipophilicity represented by a log P of 2.0 and relatively low bioavailability. The device was successfully tested in-vivo in a porcine model. The inventors demonstrate that upon adherence of the device to the inner side of the cheek, ABPD releases APO with a characteristic sustained release profile with a steady plateau drug levels in plasma within 30 min after administration, and for at least at least 8 h up to ABPD removal - a therapeutic window of at least 8 h. APO bioavailability from the buccal device was estimated in the range of 55% to 80% as opposed to per oral bioavailability of < 2%.

APO (or levodopa) is considered effective treatment for patients presenting symptoms of PD. The main limitation is the necessity of administering this drug via continuous parenteral or subcutaneous infusions. The present studies in the porcine model suggest that comparable therapeutically relevant APO levels in plasma may be reproduced in humans, and thus, can offer an alternative mode of APO delivery for treating PD, meant to substitute the conventional infusions. No safety issues were found.

In addition, a novel approach was adopted whereby a permeation marker, antipyrine (ANT), was incorporated into the buccal device. This enabled to explain the APO pharmacokinetic profile and achieve better understanding of APO pharmacokinetics post buccal administration. Present studies urge implementation of this method as a standard approach in future research on buccal mucosal drug delivery.

In the next series of experiments, the device was tested two models: (1) an in-vivo model in rat was investigated for APO permeability rate through murine buccal mucosa; and (2) an ex-vivo Using diffusion chamber with mounted excised porcine buccal mucosa (EXAMPLE 3). APO permeability through the buccal mucosa of the rat suggested that this model system does not reflect clinically relevant plasma levels. Nonetheless, these studies yielded several insights into the processes governing buccal delivery of actives, and APO in particular, and methods controlling them.

Permeability was assessed by a ‘cocktail approach’ using co-administration of APO and permeability markers metoprolol and atenolol at physiological pH 7.4. Several ways of modulating the delivery of APO were investigated. Lowering pH to 5.9 slowed permeability of APO and passive transcellular marker metoprolol by 6- and 2-fold, respectively. Addition of 1:1 ethanol:propylene glycol solution produced elevation of permeability rates by 4- and 3-fold, respectively. Addition of nanolipospheres highly elongated the lag time of APO and metoprolol. No changes in the permeability rates of the passive paracellular marker atenolol were observed. Simulation of the obtained APO data to estimate human buccal delivery suggested that therapeutically relevant blood levels could be obtained by the above manipulations.

In summary, these experiments showed that APO has relatively high permeability rates through buccal mucosa, and it is consistent with a passive transcellular diffusion. Lower pH impeded permeation rate and subsequently prolonged dose release. Addition of organic solvents, in contrast, increased permeation rate. Addition of lipidic nanoparticles ‘depot’ delayed delivery. More generally, it was shown that APO delivery via buccal mucosa using the devices and methods of the invention can provide a therapeutically relevant approach to substitute infusions for treating PD.

The last example focuses on the permeability of CBD via oral mucosa using the devices and pre-formulations of the invention in pig animal model (EXAMPLE 4). These experiments showed that CBD permeates oral mucosa and achieves clinically relevant plasma levels, the permeation is very slow and is suitable for prolonged delivery. Further, due to its very high lipophilicity CBD tends to accumulate inside the mucosa, resulting in continued prolonged release of CBD into the blood stream long after the removal of the device. It can be presumed that this absorption profile is characteristic of other highly lipophilic substances. Another important finding has been the importance of masking the drug inside a ‘shell’ to achieve maximal delivery through oral mucosa, or else the drug might be washed out by the salivary flow and ingested in the GI tract. This finding can be useful in terms of structural considerations in design and development of next generation delivery devices targeting oral mucosa as the primary administration site.

### BRIEF DESCRIPTION OF THE DRAWINGS

To better understand the subject matter that is disclosed herein and to exemplify how it may be carried out in practice, embodiments will now be described by way of non-limiting examples with reference to the accompanying drawings.

FIG. 1 illustrates certain embodiments of the controlled delivery device of the invention. Three layer tablets were prepared by compression molding loading first the mixture of 20-50 mg Carbopol (CP) or Carbopol:Hydroxypropyl methylcellulose (CP:HPMC); on top of this layer, 100 mg CP:HPMC powder with 10% (w/w) glycerol; and another layer of adhesive powder 20 to 50 mg CP or CP:HPMC mixture without glycerol; the powder was compressed into tablets.

FIG. 2 illustrates the effect of glycerol content on the flexibility properties of the devices and films of the invention.

FIG. 3 shows the results of flexibility test, wherein devices of the invention with various glycerol content were subjected to elevating degrees of deformation or bending force. The devices containing 7% glycerol were significantly more flexible than those without the glycerol, regardless of the exerted force.

FIGS. 4A-4B show release profiles from devices containing various types of herbal extracts (Salvia, Echinacea Rozmarin) detected at A: 280 nm and B: 320 nm. The results indicate continuous prolonged release of actives within the experimental timeframe 8 h.

FIG. 5 shows the effect of phenol lyophilization on the release profile from the devices of the invention, suggesting contribution of phenol lyophilization by about 10%.

FIG. 6 shows release profiles from the devices of the invention containing various types of actives (from left to right: Dyclonine, Salvia, Rozmarin, Echinacea, Lemon oil, Eucalyptus oil) and phenol lyophilization (last column).

FIG. 7 illustrates the effect of glycerol on the flexibility of the devices (tablets). Smooth and rough devices were produced with the basic formula 80% CP and 20% HPMC and 0%, 10% and 20% glycerol. Figure shows the difference between hard and flexible tables, suggesting that the addition of glycerol leads increased flexibility.

FIGS. 8A-8D illustrate the transparency and flexibility features of the devices with various content of polymers with the basic formula of 90% w/w polymer mixture and glycerol, and specific examples of A: polyvinylpyrrolidone, B: poly(vinyl alcohol), C: poly(acrylic acid) and D: hydroxypropyl cellulose, and 10% w/w glycerol.

FIG. 9 illustrates the durability feature of the devices (films) as a function of time under conditions mimicking the mouth moisture and temperature (phosphate buffer (pH 6.8) at 37° C. The medium was changed every 10 min and saved for the determination drug content (dyclonine). Pictures show that during the first 30 min, the films were intact and constantly releasing dyclonine. After 30 min, the films started to disintegrate.

FIG. 10 illustrates the effect of the total amount of the components on the thickness on durability of the devices. The devices were made of 71.2% CP, 8.9% HPC, 8.9% carrageenan (CGN), 1% dyclonine and 10% glycerol (w/w), compressed and molded to contain various amounts of the mixture (80 mg, 160 mg and 240 mg) yielding films with various thickness (180 µm, 360 µm and 530 µm, respectively). The thinnest film (80 mg) was more flexible and had better transparency compared to thicker films (160 mg and 240 mg). All films survived 20 bending tests.

FIGS. 11A-11E illustrate the durability, transparency and flexibility features with various polymers using the basic formula 90% polymer mixture and specific examples of A: Microcystalline cellulose, B: Carboxymethyl cellulose (CMC), C: Eudragit, D: Pullulan and E: Pectin, and 10% glycerol. Picture show various degrees of brittleness and transparency depending on the type of polymer or polymer mixture.

FIG. 12 shows release profiles from devices made of Polyvinylpyrrolidone (PVP) or Poly(acrylic acid CP and 2,4 dichlorobenzyl alcohol. Both PVP and CP films show continuous prolonged release over the period of at least 25 min.

FIG. 13 shows adhesion properties of devices made of Polyvinylpyrrolidone (PVP), Poly(acrylic acid (CP) or k-carrageenan (CGN), indicating that films with PVP and CP have superior adhesion properties.

FIGS. 14A-14B shows semi-logarithmic plots of plasma concentrations of apomorphine (APO) and APO metabolites vs time in experimental animals (pigs) following IV administration of 1.0 mg/kg dose of 10 mg/ml APO in 1.0-1.5 min infusion. Mean (±SEM), n = 4. A: (●) APO (plasma concentration in ng/ml). B: (o), (□), and (x) APO quinone, APO glucuronide, and APO sulfate, respectively (plasma concentrations as a ratio of peak areas metabolite to internal standard obtained from chromatogram).

FIG. 15 shows a proportion of APO released from the hydrogel at pH 5.9 into aqueous solution vs time through dialysis bag. 20 mg APO in 0.5 ml hydrogel with gelatin concentrations of (●) - 0%^{w}/_{w}, (x) - 10%^{w}/_{w}, (o) - 12%^{w}/_{w} and (□) - 15%^{w}/_{w}. Mean (±SD), n = 3.

FIG. 16 shows (●) plasma APO concentration vs. time (mean ±SEM) following APO administration from buccal prolonged release delivery device containing ~2.0 mg/kg dose of 38.30 mg/ml APO in hydrogel-based core with exposed area for absorption of 4.74 cm² to four pigs with average weight of 43 kg. (o) a device containing 1.37 mg/kg dose of 18.80 mg/ml APO in hydrogel core with exposed area for absorption of 7.84 cm² (n = 1). The arrow indicates the time when the device was removed.

FIG. 17 shows semi-logarithmic plots of concentration vs. time (mean ± SEM) following administration of the buccal prolonged release delivery device with hydrogel-based core containing (●) ~2.0 mg/kg dose of 38.30 mg/ml APO, and (o) ~0.05 mg/kg dose of 0.99 mg/ml antipyrine (ANT). Exposed area for absorption = 4.74 cm², n = 4. The arrow indicates the time when the device was removed.

FIGS. 18A-18C show representative histopathological slides of porcine buccal mucosa stained with hematoxylin and eosin. A: buccal mucosa exposed to HPC:CP-based shell device for 8 h; B: buccal mucosa exposed to hydrogel-based core device for 8 h; C: non-treated buccal mucosa. A-B: The cheek exposed to the devices was facing down during the experiment (the side on which the pig was lying), C: the non-treated cheek was facing up. N and arrow show neutrophil aggregation.

FIG. 19 shows mean (±SD) apparent permeability (Papp) of ANT, metoprolol and APO through excised porcine buccal mucosa in Ussing diffusion chamber from simulated saliva at pH 7.4, pH 5.9, or pH 7.4 with of 3.33%^{v}/ᵥ 1:1 ethanol: propylene glycol solution (n=3). Two-way ANOVA with Tukey post hoc analysis. p-value < 0.05: (*) compared to other two test substance in the same intervention group, (x) compared to the pH 7.4 group for the same test substance.

FIG. 20 shows cumulative amounts permeating through excised porcine buccal mucosa in Ussing diffusion chamber from simulated saliva at (●) pH=7.4 vs (□) pH=7.4 with addition of 10%^{v}/ᵥ nanoliposphere formulation (n=3) (mean ±SD).

FIG. 21 shows mean (±SD) APO Papp through excised porcine buccal mucosa in Ussing diffusion chamber from donor chamber containing standard test concentration vs 100-fold higher concentration (n=3).

FIG. 22 shows APO concentration in plasma vs time following IV administration of 2 mg/ml to rats, mean (± SEM), n=3.

FIG. 23 shows (●) Cannabidiol (CBD) and (o) theophylline (TPH) plasma concentrations vs. time (mean ±SEM) from the devices containing CBD ~0.42 mg/kg dose of 30.0 mg/ml and TPH ~0.11 mg/kg dose of 8.0 mg/ml dissolved in 1:1 ethanol: propylene glycol solution, with exposed area for absorption of 1.58 cm² to three experimental animals. The arrow shows the time of removal from the mucosa.

### DETAILED DESCRIPTION OF EMBODIMENTS

In the broadest aspect the invention provides a solvent-free device for controlled delivery of at least one poorly water-soluble or lipophilic active through at least one tissue of the oral cavity of a subject, the device being a solid fast dissolving device comprising at least one water-soluble or hydrophilic polymer and at least one edible alcohol; and the at least one atomized poorly water-soluble or lipophilic active comprised in a solid pre-formulation with at least one dispersant, at least one lipid

Under water-soluble or hydrophilic polymer is usually meant polymers which dissolve in, or are swollen by, water. Notable examples of hydrophilic polymers are proteins, cellulose, polyethylene glycol ethers, polyamides, polyacrylic amides, polyurethanes with polyethylene glycol ether soft segments, ethoxylated graft polymers. Hydrophilic polymers not only feature significant properties in the dissolved state, but also as crosslinked materials, such as in hydrogels. Some polymers are hydrophilic but not water-soluble but rather water-swellable, such as poly(2-hydroxyethyl methacrylate).

In numerous embodiments the hydrophilic polymers comprised in the devices of the invention are poly(carboxylic acids).

The in numerous embodiments the invention can be articulated as a solvent-free device for controlled delivery of at least one poorly water-soluble or lipophilic active through at least one tissue of the oral cavity of a subject, the device being a solid fast dissolving device comprising at least one poly(carboxylic acid) and at least one edible alcohol; and the at least one atomized poorly water-soluble or lipophilic active comprised in a solid pre-formulation with at least one dispersant, at least one lipid component and at least one amphiphilic material.

The term ‘a solvent-free’ herein refers to the property of the device as having no or a little proportion of solvent(s) to the extent of up to 0.001%, 0.005%, 0.01%, 0.05%, 0.1%, 0.5%, 1%, 5%, 10%, 15% and 20% solvent (w/w) of the total weight, or more specifically, in the range of at least about 0.001%-0.005%, 0.005%-0.01%, 0.01%-0.05%, 0.05%-1%, 1%-5%, 5-10%, 10-15%, 15-20% (w/w) and more of the total weight.

The term ‘fast dissolving’ herein refers to the property of the device to dissolve or disintegrate in the oral cavity to the extent in the range of about 10%-20%, 20%-30%, 30%-40%, 40%-50%, 50%-60%-, 60%-70%, 70%-80%, 80%-90%, 90%-100% of the total weight during a period in the range of about 1-5 min, 5-10 min, 10-15 min, 15-20 min, 20-30 min, 30-40 min, 40-50 min, 50-60 min, and further up to 1 h, 2 h, 3 h, 4 h, 5 h, 6 h, 7 h, 8 h, 9 h, 10 h after administration. The term also defines a period of dissolution that is immediate or over a period about 1-5 min, 5-10 min, 10-15 min, 15-20 min, 20-30 min, 30-40 min, 40-50 min, 50-60 min, or further up to 1 h, 2 h, 3 h, 4 h, 5 h, 6 h, 7 h, 8 h, 9 h, 10 h after administration.

The term ‘controlled delivery’ is used herein in its broadest sense to denote a formulation or a method whereby the permeation of active or drug through tissues, its accessibility and bioavailability in tissues and blood circulation, and/or targeting to the specific tissues of action are modulated to achieve specific effects. It encompasses immediate, prolonged, and sustained delivery of actives or drugs, drug protection against degradation, preferential metabolism, clearance or delivery to specific tissues. Controlled release of actives included in a device of the invention can be obtained by several means. In the first example, less swellable polymeric carriers such as HPMC (hydroxypropyl methyl cellulose) may be used. Second possibility is to form a preformuation of the active agent in a controlled release system and incorporate same into the formulation. Third possibility is to increase the hydrophobicity of the formulated device by adding hydrophobic agents such as fatty acids, triglycerides, wax etc. Any of these and others known in the art may be utilized.

The term ‘immediate delivery’ implies an immediate permeation and/or release of active into an oral tissue or circulation, or in other words, that the active can be detected or measured in the tissue or circulation within a relatively short period of time, and in this case, within a period of up to at least about 1, 2, 3, 4, 5, 6, 7, 8, 9, 10 min, and further after up to at least about 15, 20, 15, 30, 35, 40, 45, 50, 55 and 60 min after administering the device of the invention to the oral cavity. The term further applies to immediate release of active in the target organs and tissues although with a slightly delayed timing or a lag, such as within at least about 5, 10, 20, 30, 40, 50, 60, 70, 80, 90, 100, 110, 120, 130, 140, 150, 160, 170, 180 min or more after administering the device to the oral cavity.

The term ‘prolonged delivery’ implies a delayed permeation and/or release of active into an oral tissue or circulation, or in other words, that the active can be detected or measured in the tissue or circulation after a lag period, and in this case, after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering the device to the oral cavity. The prolonged delivery also applies to target organs and tissues with additional lag of at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering the device to the oral cavity or relative to the first detection of the active in the oral tissue or circulation.

The term ‘sustained delivery’ implies a profile of continued permeation and/or release of active into an oral tissue or circulation from the device of the invention, or in other words, that the permeation and/or release of the active into the tissue or circulation reaches a plateau or a steady state after at least about 10, 20 30, 40, 50, 60, 70, 80, 90, 100, 110, 120, 130, 140, 150, 160, 170, 180 min and further after at least about 3 h, 4 h, 5 h, 6 h, 7 h, 8 h, 9 h, 10 h or more after administering the device to the oral cavity, and that the plateau or the steady state persists for at least about 1 h, 2 h, 3 h, 4 h, 5 h, 6 h, 7 h, 8 h, 9 h, 10 h, 11 h, 12 h, 13 h, 14 h, 15 h, 16 h, 17 h, 17 h, 18 h, 19 h, 20 h or more after administering the device to the oral cavity or relative to the first detection of the active in the oral tissue or circulation.

The terms ‘poorly water-soluble’ or ‘lipophilic’ (hydrophobic) in the context of present invention refer to actives or drugs characterized with water-solubility in the range of about 0.1-0.5 mg/ml, 0.5-1 mg/ml, 1-5 mg/ml, 5-10 mg/ml, 10-15 mg/ml, 15-20 mg/ml in neutral pH.

The term ‘tissue of the oral cavity’ encompasses herein mucous membrane linings or epithelia of the oral cavity and organs of the oral cavity and muscle tissues. It further encompasses epithelia of the lips, gingivae, retromolar trigone, teeth, hard palate, cheek mucosa, mobile tongue, dorsal tongue mucosal tissue, floor of the mouth, the major salivary glands and/or esophagus.

The term ‘atomized’ in connection with the poorly water-soluble active refers herein to the particulate nature of active in the solid pre-formulation due to the presence of dispersant(s), lipid component(s) and amphiphilic material(s) and/or mechanical methods. This term further implies a reduction of particle size of a material to a nanometric and/or a micrometric range. An atomized material can have particle size in a range between about 5 to 1,000 nm or in the range between about 5 nm to 10 µm and more, or in the range between about 1-100 nm, 100-200 nm, 200-300 nm, 300-400 nm, 400-500 nm, 500-600 nm, 600-700 nm, 700-800 nm, 800-900 nm, 900-1000 nm, and further in the range between about 1-10 µm, 10-20 µm, 20-30 µm, 30-40 µm, 40-50 µm, 50-60 µm, 60-70 µm, 70-80 µm, 80-90 µm 90-100 µm.

In some embodiments, the atomized particles are of a size between 1 nm and about 1,000 nm or between 20 nm and 500 nm.

In some embodiments, the size of the atomized particles is in the nanometric scale.

In numerous embodiments the at least one atomized poorly water-soluble or lipophilic active comprised in a solid pre-formulation can have particle size in a range between about 1 to 500 nm, or more specifically in the range between about 1-10 nm, 10-20 nm, 20-30 nm, 30-40 nm, 40-50 nm, 50-60 nm, 60-70 nm 70-80 nm, 80-90 nm or 90-100 nm and further in a range between about 100-150 nm, 150-200 nm, 200-250 nm, 250-300 nm, 300-350 nm, 350-400, 400-450 nm or 450-500 nm.

In other embodiments, the atomized poorly water-soluble or lipophilic active may have a size that is between 20 and 250 nm, 20 and 200, 20 and 190, 20 and 180, 20 and 170, 20 and 160, 20 and 150, 30 and 200, 30 and 150, 40 and 200, 40 and 150, 50 and 200, 50 and 150 or 50 and 100 nm.

In some embodiments, the atomized poorly water-soluble or lipophilic active may have a size that is between 10 and 100 nm or between 30 and 200 nm or between 50 and 150 nm.

More specifically, the formulation of the invention is configured in a way that upon dispersion in aqueous media it spontaneously forms a dispersion of the active, wherein the active is contained within dispersed nanodroplets or nanovehicles that facilitate the penetration of active through tissues and increase its bioavailability in tissues and blood circulation.

Thus, the formulation of the invention can be regarded as a pro-nanodispersion system (PNS).

The dispersed nanodroplets or nanovehicles can be in various sizes in the range between about 1 nm and about 50 nm, or is between about 50 nm and about 100 nm, between about 100 nm and about 150 nm, between about 150 nm and about 200 nm, between about 200 nm and about 250 nm, between about 250 nm and about 300 nm, between about 300 nm and about 350 nm, between about 350 nm and about 400 nm, between about 400 nm and about 450 nm, between about 450 nm and about 500 nm, between about 500 nm and about 550 nm, between about 550 nm and about 600 nm, between about 600 nm and about 650 nm, between about 650 nm and about 700 nm, between about 700 nm and about 750 nm, between about 750 nm and about 800 nm, between about 800 nm and about 850 nm, between about 850 nm and about 900 nm, between about 900 nm and about 950 nm or between about 950 nm and about 1000 nm.

It should be noted that in numerous embodiments the devices of the invention can further provide delivery of water-soluble actives. This type of actives usually do not require use of PNS, but are rather mixed and embedded directly with the components of the device, i.e. at least one poly(carboxylic acid) and at least one edible alcohol.

In other words, in numerous embodiments the invention provides a solvent-free device for prolonged delivery of at least one water-soluble or hydrophilic active through at least one tissue of the oral cavity of a subject, the device being a solid fast dissolving device comprising at least one water-soluble polymer and at least one edible alcohol and at least one water-soluble active.

With respect to the general properties, the devices of the invention can take any shape or form, or a form that is substantially circular, oval, square or rectangular.

In numerous embodiments the devices of the invention can be in the form of a tablet or a film. The tablet compared to the film is three dimensional.

In other embodiments the devices of the invention can be substantially two dimensional, meaning the thickness of the device is very low; or that two sides with the largest lengths are about x10, x20, x30, x40, x50, x60, x70, x80, x90, x100, x200m x300, x400, x500, x600, x700, x800, x900, x1000 larger, and more, than its thickness.

In numerous embodiments the shape and the size of the devices are compatible with the size and architecture of the oral cavity.

In certain embodiments the devices of the invention can have a surface area in the range between about 1 and about 4 cm².

In other embodiments the devices of the invention can have at least one side with a surface area in the range between about 1 and about 4 cm².

In numerous embodiments the devices of the invention adhere to at least one tissue of the oral cavity.

In numerous embodiments the devices of the invention adhere to a mucous membrane lining of at least one organ of the oral cavity, for example buccal mucosa or hard palate mucosa, or sublingual mucosa.

In other embodiments the devices can adhere to a muscle tissue of at least one organ of the oral cavity, for example the tongue.

In numerous embodiments the devices are configured so as to adhere with a side of the larger surface area, whether the device is a tablet or a film.

In certain embodiments the devices can be one-sided, meaning that they are configured so as to adhere to the tissue and release the active from the same side.

In other embodiments the devices can be two-sided, meaning that they are configured so as to adhere to the tissue and release the active from two different sides. This type of devices is also referred to herein as asymmetric devices.

Significant effort has been made to develop bioadhesive polymers for prolonging contact time in mucosal routes of drug administration. The ability to maintain a delivery system at a particular location for extended periods of time has many advantages for both local oral disease treatment as well as systemic drug bioavailability. As mentioned above, relative accessibility and specific properties of the oral mucosa make it an attractive site for local and systemic drug delivery.

In numerous embodiments the devices of the invention can provide controlled delivery of one or more actives through the mucosal tissue and thereby a systemic delivery of the active(s). The oral mucosal surfaces are usually rich in blood supply, providing the means for rapid drug transport to the systemic circulation.

In other words, in such cases the controlled delivery further comprises a systemic delivery of said poorly water-soluble or lipophilic active(s).

In other embodiments the devices of the invention can provide targeted delivery of active(s). The term ‘targeted’ encompasses herein local delivery in terms of tissues and organs of the oral cavity, and further systemic delivery targeted to specific organs wherein the active or drug is intended to be effective.

Thus, the concepts of systemic and targeted delivery are not necessarily mutually exclusive and in certain cases are overlapping in referring to different properties of the same drug.

Thus, in numerous embodiments the devices of the invention can provide systemic delivery of the at least one poorly water-soluble or lipophilic active which further comprises a targeted delivery of said poorly water-soluble or lipophilic active(s) to at least one tissue or an organ of the human body.

In numerous embodiments said controlled delivery of the at least one poorly water-soluble or lipophilic active comprises an immediate, a prolonged and/or a sustained delivery of said poorly water-soluble or lipophilic active(s) to at least one target tissue or target organ.

In other embodiments the devices of the invention can provide controlled delivery of the at least one poorly water-soluble or lipophilic active which is a targeted delivery of active(s) to at least one tissue or an organ of the oral cavity.

In numerous embodiments said targeted delivery is directed to a mucous membrane lining of at least one organ of the oral cavity and/or a muscle tissue of at least one organ of the oral cavity.

In other embodiments said targeted delivery is directed to at least one organ of the oral cavity, e.g., the lips, gingivae, retromolar trigone, teeth, hard palate, cheek mucosa, mobile tongue, dorsal tongue mucosal tissue, floor of the mouth, the major salivary glands and/or esophagus.

Transmucosal oral drug delivery is used to treat a range of diseases of the oral cavity. Common examples are dental diseases such as dental caries, which are usually treated by minimal dental interventions and preventive fluoride therapy. Additional examples are dry mouth, ulcers, fungal, viral, and bacterial infections in the mouth, gums and throat, and non-infectious throat inflammations, the classical treatments of which are lozenges, drops, sprays and solutions containing antiseptics such as phenol derivatives, tetracycline, triclosan, organic zinc salts, and pain relievers such as benzocaine and lidocaine and natural substances such as menthol, natural extracts, and essential oils.

The main limitation with the existing drugs is that the time of exposure to actives is restricted to the time that the active remains in the mouth - 10 min max. The mucoadhesive devices of the invention provide a successful solution to this problem in the form of mucoadhesive and intraoral stickers, tablets, or films.

In terms of chemical composition of the devices of the invention, in numerous embodiments the devices comprise at least one poly(carboxylic acid) and at least one edible alcohol.

In numerous embodiments devices further comprise at least one polyol.

In further embodiments the at least one polyol is selected from the group of hydroxy propyl cellulose, hydroxypropyl methyl cellulose and their corresponding amylose derivatives, polyvinyl alcohol, natural gums, and chitosan.

In numerous embodiments the at least one poly(carboxylic acid) is at least one polyacrylic acid.

In other embodiments the at least one polyacrylic acid is at least one crosslinked polyacrylic acid.

In numerous embodiments the at least one polyacrylic acid is selected from the group of crosslinked polyacrylic acids and methacrylic acids, copolymers thereof, alginates, hyaluronic acid, carboxymethyl cellulose, and mixtures thereof.

In certain embodiments the devices of the invention comprise a crosslinked polyacrylic acid and hydroxypropyl cellulose.

In numerous embodiments the content of the crosslinked polyacrylic acid(s) is in the range of between about 40% and about 99% per total weight (w/w), or more specifically in the range between about 30%-99%, 35%-99%, 40%-99%, 45%-99%, 50%-99%, 55%-99%, 60%-99%, 65%-99%, 70%-99%, 75%-99%, 80%-99%, 85%-99% and 90-99% (w/w) of the total weight, or up to at least about 40%, 45%, 50%, 55%, 60%, 65%, 70%, 75%, 80%, 85%, 90%, 95% and 99% (w/w) of the total weight.

In numerous embodiments the at least one edible alcohol is a small alcoholic molecule.

In certain embodiments the at least one edible alcohol is selected from the group of ethanol, isopropanol, glycerol, monoglycerides, ethyl lactate, polyethylene glycols, and tributyl citrate.

In numerous embodiments the content of the edible alcohol(s) is in the range of between about 1% and about 20% per total weight (w/w), or more specifically in the range between about 1%-20%, 2%-20%, 4%-20%, 6%-20%, 8%-20%, 10%-20%, 12%-20%, 14%-20%, 16%-20%, and 18%-20% (w/w) of the total weight, or up to at least about 1%, 2%, 4%, 6%, 8%, 10%, 12%, 14%, 16%, 18%, 20%, 25% and 30% (w/w) of the total weight.

In numerous embodiments the devices of the invention comprise at least one crosslinked polyacrylic acid which is Carbopol and least one edible alcohol which is glycerol.

One of the important features of the devices of the invention is the ability to control the flexibility and the texture of the devices, and thereby their accessibility and adhesiveness to the oral cavity and oral cavity organs and tissues.

Flexibility can be measured by various known in the art methods. In many cases, and especially for substantially two-dimensional devices or films, the flexibility of is measured by the angle of joints, or the range of motion thereof (ROM).

The flexibility feature is particularly important in view of the known limitations of the currently available mucoadhesive tablets or films, and specifically rigidity and low adhesiveness to the mucosal tissue, and as a result, poor patient compliance due to inconvenience and/or ineffectiveness of the device or the treatment overall. These are some reasons why only few of the existing devices have succeeded to reach the market.

In numerous embodiments the devices of the invention can have a flexibility measured by the angle of joints or ROM in the range of between about 10° and about 50°, or more specifically in the range between about 5°-50°, 10°-50°, 15°-50°, 20°-50°, 25°-50°, 30°-50°, 35°-50°, 40°-50° and 45°-50°, or up to at least about 5°, 10°, 15°, 20°, 25°, 30°, 35°, 40°, 45°, 50°, 55°, 60°.

In numerous embodiments, the devices of the invention can have a flexibility measured by the angle of joints or ROM of at least about 30° or more specifically at least at least about 5°, 10°, 15°, 20°, 25° and 30°.

In numerous embodiments the flexibility of the device is controlled by the content of the edible alcohol per total weight (w/w).

Another important feature of the devices of the invention is preferential solubility in the oral cavity over a predetermined period of time. This feature has been referred to above as a ‘fast dissolving’ property of the device.

In numerous embodiments a preferential solubility of the devices of the invention can be in the range of between about 30% and about 100% of the total weight being solubilized in the oral cavity over a period in the range between about 1 min and 60 min, or more specifically in the range between 30%-40%, 40%-50%, 50%-60%-, 60%-70%, 70%-80%, 80%-90%, 90%-100% of the total weight during a period in the range of about 1-5 min, 5-10 min, 10-15 min, 15-20 min, 20-30 min, 30-40 min, 40-50 min, 50-60 min.

In certain embodiments a preferential solubility of at least 20% of the total weight being solubilized in the oral cavity over a period in the range between about 1 min and 60 min. or more specifically up to at least about 5%, 10%, 15%, 20% of the total weight over a period in the range between about 1-5 min, 5-10 min, 10-15 min, 15-20 min, 20-30 min, 30-40 min, 40-50 min, 50-60 min.

Yet another important feature of the devices of the invention is controlled adherence or adhesiveness to at least one tissue or organ of the oral cavity over a predetermined period of time.

In numerous embodiments the devices of the invention can have a controlled adherence or adhesiveness in the range of between about 30% and about 100% of a surface with the largest area adhering to at least one tissue or organ of the oral cavity over a period in the range between about 1 min and 60 min, or more specifically in the range between about 20%-100%, 30%-100%, 40%-100%, 50%-100%, 60%-100%, 70%-100%, 80%-100%, 90%-100% of a surface with the largest surface area adhering to at least one tissue or organ of the oral cavity over a period in the range between about 1-5 min, 5-10 min, 10-15 min, 15-20 min, 20-30 min, 30-40 min, 40-50 min, 50-60 min, and over 1 h, 2 h, 3 h, 4 h, 5 h, 6 h, 7 h, 8 h, 9 h, 20 h and more.

In certain embodiments the devices can have a controlled adherence or adhesiveness of at least about 80% of a surface with the largest area adhering to at least one tissue or organ of the oral cavity over a period in the range between about 1 min and 60 min, or more specifically of at least about 10%, 20%, 30%, 40%, 50%, 60%, 70% and 80% of a surface with the largest surface area adhering to at least one tissue or organ of the oral cavity over a period in the range between about 1-5 min, 5-10 min, 10-15 min, 15-20 min, 20-30 min, 30-40 min, 40-50 min, 50-60 min, and over 1 h, 2 h, 3 h, 4 h, 5 h, 6 h, 7 h, 8 h, 9 h, 20 h and more.

In numerous embodiments the controlled adherence or adhesiveness on the two sides of the device with the largest surface area are substantially similar.

In other embodiments the controlled adherence or adhesiveness on the two sides of the device with the largest surface area are distinct.

In numerous embodiments at least one side of the device with the largest surface area has a rough texture.

In other embodiments at least one side of the device with the largest surface area has a smooth texture.

In numerous embodiments the side with a rough texture has a better adherence or adhesiveness to at least one tissue or organ of the oral cavity compared to the side with a smooth texture.

In further embodiments the side with a rough texture has a lower content glycerol per total weight (w/w) compared to the side with a smooth texture.

In practical terms, polyol that are suitable for tablets and stickers of the invention are hydroxy propyl cellulose (HPC), hydroxypropy methyl cellulose (HPMC) and their corresponding amylose derivatives, polyvinyl alcohol, hyaluronic acid, chitosan, arbinogalactan, guar gum, dextran, cyclodextrin, sulfated and non-sulfated carrageenan and other gums and polysaccharides.

Polycarboxylic acid that are suitable for adhesion include crosslinked poly acrylic acid and methacrylic acid and their copolymers, alginates, hyaluronic acid, carboxymethyl cellulose and their mixtures.

The most suitable compositions are those made from crosslinked poly(acrylic acid) and hydroxypropyl cellulose.

Poly(acrylic acid) (PAA, known as Carbopol or Carbomer) is generic name for synthetic high molecular weight polymers of methacrylic and acrylic acid. They may be homopolymers of acrylic acid, crosslinked with an allyl ether pentaerythritol, allyl ether of sucrose or allyl ether of propylene. In a water solution at neutral pH, PAA is an anionic polymer with the ability to absorb and retain water and swell to many times their original volume, depending on the degree of crosslinking. Dry PAAs are found in the market as white and fluffy powders. Carbopol or Carbomer codes (910, 934, 940, 941 and 934P) are an indication of molecular weight and the specific components of the polymer. For many applications PAAs are used in form of sodium salt or ammonium salts. Maleic acid copolymers; polysaccharides such as karaya gum, tragacanth gum, xanthan gum, jaraya gum, pectin, guar gum, locust bean gum, psyllium seed gum, alginates, hydrocolloid gels prepared from polysaccharides extracted from Fronia elephantum, Sapindus trifoliatus, Kunjac, and the cashew tree; In some embodiments, the composition is a double layer tablet containing a bioadhesive layer. The bioadhesive materials in the bioadhesive layer are present in 10% to 100% by weight of the bioadhesive layer, preferably from about 10% to 50% by weight of the bioadhesive layer, most preferably about 45% by weight of the bioadhesive layer.

Stickers of different weights/thickness, compositions and surface roughness can be prepared and tested for adhesion to oral cavity tissues. In a typical setup, a sticker is pushed against saliva free buccal surface or the tongue. Salvia free tongue can be achieved simply by swelling salvia prior to application and held with pressure for 10 sec. Adhesion is tested by the ability to remove the sticker. Double adhesion can be also examined by first adhering the sticker to one surface, either the tongue or the front inner side of the lip, while keeping the other side of the sticker dry, pushing the other surface against the saliva free tissue and hold with pressure for 10 sec to allow adhesion. The ability to detach the tongue from the other surface can be tested. As per existing experience, the sticker composition with a better adhesion to soft tissue surfaces is with a higher PAA (Carbopol).

As for the method of preparation, dry powders of Carbopol 940 and HPC at a weight ratio of 70:30, 80:20 and 95:5 is mixed and round tablets of 14 mm in diameter are prepared by compression molding of 100, 150 and 200 mg mixture for each tablet at a pressure of 5 tones/cm² for 2 sec. The metallic mold discs that are in contact with the powder during compression are of flat smooth surface or have different roughness, the surface roughness is obtained by curving crisscross lines at different depth and distance. Roughness of 15, 30, 50 and 100 µm depth have been prepared. For minor surface roughness, the surface of the disc is rubbed with high quality sandpaper that affect stainless steel.

It should be noted that, so far, little attention has been given to the preparation of films by compression molding, most likely due to their limited flexibility and tendency to break upon folding and de-folding. Nonetheless, compression molding of powders or powder blends is still interesting as it offers a low cost, fast and adaptable process that can be used in common tableting machines: it can be performed at room temperature with no solvents; produce films of any size, thickness and shape, depending on the mold design; and produce asymmetric films with versatile thicknesses and layering of different materials to provide asymmetric absorption of water or asymmetric release of drugs.

The size of the sticker (tablet or film) can range from 2 to 25 mm in diameter and 1 to 6 mm in thickness. Solid and fast dissolving stickers can be prepared with various flexibilities and adhesion properties, and further, to include various types of actives for local and systemic applications.

### Preparation of Flexible Stickers

A typical preparation of a flexible sticker uses a mixture of Cabopol 934 (150 mg) and HPC (50 mg) powder, 10, 20, 40 or 80 µl of ethanol, polyethylene glycol 400 or glycerol are added to about 50 mg powder, and mixed to form a dough-like material, the remaining powder was gradually added and mixed well to form uniform granules. The material is compressed into stickers as described above to form almost clear flexible tablets to stickers, depending on the content of alcohol. Full flexibility can be achieved with a higher content of alcohol up to over 135° bending without break. The adhesion capacity is not affected by alcohol contents. To prepare stickers with lower alcohol content, 40 mg dry powder of the mixture is first added to the compression device, followed by the addition of the alcohol containing granules (160 mg) and compressed into a tablet that is flexible for using with a less flexible surface. Alternatively, granules are mixed with the dry powder at different ratio and compressed into stickers of uniform compositions.

Flexible stickers are also prepared by exposing stickers prepared from dry powder without any alcohol-to-alcohol vapors in a chamber. The exposure time and chamber size and temperature determine to absorption of alcohol into the sticker. This process allows non-symmetric flexibilities and properties.

Stickers made with alcohol are packed in aluminum foil pockets to retain the alcohol. The flexibility retains for days when exposed to room temperature.

Compounds that are suitable as plasticizers are safe edible alcohols, including ethanol, isopropanol, glycerol, monoglycerides, ethyl lactate, polyethylene glycols, and tributyl citrate.

### Preparation of Flexible Stickers for Intra Oral Delivery of Agents

In a typical protocol: 150 mg of granules made from a mixture of glycerol, Carbopol 934, and HPC (40:150:50 w/w) are mixed with active agent at the amount of 5-50 mg and compressed into tablets or strips using a rolling continuous press. The size and shape of the sticker is designed by the punch shape or cut the strip into certain sizes and shapes. Active agents incorporated in these stickers can include agents for treating halitosis, fungal and microbial infections, viral infections, gingivitis, dental etching, sore throat, mouth refreshing, oral ulcers and for systemic delivery of agents, and further citrus oil, herbal extracts, camphor, flavors, cannabinoids, povidone-iodine complex, nicotine, caffeine, antimicrobial, amphotericin B, methadone, oxycodone, morphine, homeopathic agents, vitamins (e.g., vit. E, C, B₁₂), zinc oxide, herbal extracts, and local anesthetics such as lidocaine, bupivacaine, opioids, antidepressants, and more.

### Asymmetric Double-Sided Mucoadhesive Tablets

There is a great need for a solid device that is capable of temporarily adhering two different tissues within the oral cavity such as the buccal surface, tooth, upper and lower parts of the tongue, plate and lips. The device is in a hard or flexible disc or film composed of materials commonly used in oral pharmaceutical formulations that dissolve or dispersed over time in the oral cavity. The devices can have a controlled degree of adhesive strength and duration of adhesion, tailored to the specific pair of tissues for a specific medical need.

Anisotropic devices in the form of a thin film, patch or disc of different shapes, compositions, thickness, roughness and elasticities that possesses different adhesion capacities in each side of the device are subject of this invention.

Surfaces of tissues in the oral cavity are composed of different outer cell types and possess different properties. The tongue is a muscular organ in the mouth covered with moist, pink tissue called mucosa and tiny bumps called papillae give the tongue its rough texture. The lower side of the tongue is composed of a different tissue with a different texture. The buccal consists of a smooth mucosal tissue of different consistency and composition. The tooth is a hard tissue smooth and non-mucosal.

The factors affecting bio-adhesion strength and duration to oral surfaces include the surface properties of the target tissue and the properties of the adherent device. Each tissue in the oral cavity can have a specifically tailored bio-adhesive tablet or sticker.

Regarding the device, surface roughness, composition, intimate contact, flexibility of device, movement of the tissues and forces applied on the device, are all affecting the adhesion strength and duration of adhesion. As per existing experience, roughness and composition of the device have strong effect on the adhesion properties to the tongue, but much less on mucosal adhesion.

Thus, in numerous embodiments the devices of the invention can be buccal devices. Under ‘buccal’ is meant that the devices can be positioned on or adhered to the mucosal lining on the inner side of the cheek.

In other embodiments the devices of the invention can be sublingual devices, meaning that they can be positioned on or adhered to the mucosal lining under the tongue.

In certain embodiments the devices of the invention can be positioned on or adhered to at least one mucosal tissue in the oral cavity.

In other embodiments the devices of the invention can be positioned on or adhered to the dorsal tongue mucosal tissue or to the muscle of the tongue.

In terms of actives, the devices of the invention can comprise a wide range of actives for clinical and not strictly clinical purposes. The devices can further comprise combination of active that are compatible in terms of solubility and biological activity.

In general terms, the applicable actives encompassed by the FDA approved drugs and FDA regulated food additives generally recognized as safe (GRAS).

In numerous embodiments the devices of the invention can comprise actives selected from the group of analgesics, anesthetics, antiseptics, antibacterial agents, antiviral agents, disinfectants, herbal extracts, anti-halitosis agents, anti-inflammatory agents, opioids, cardiovascular drugs, caffeine, nicotine, mood stabilizing or stimulating agents and antidepressants.

In numerous embodiments the devices of the invention can comprise biological actives selected from the group of peptides, proteins, enzymes, and single- or doublestranded nucleic acids.

In numerous embodiments the devices of the invention can comprise actives selected from the group of small and peptide drugs, herbal and animal extracts, homeopathic agents, nutraceuticals, vitamins, probiotic bacteria, and combination thereof.

As has been noted, the devices of the invention are advantageous for the incorporation of poorly water-soluble or lipophilic actives owing to the use of a solid pre-formulating of dispersant(s), lipid component(s) and amphiphilic material(s) (PNS), whereby said actives are atomized and presented in a form for controlled delivery.

In numerous embodiments the at least one dispersant is selected from Tween, Span, phospholipids, polyethylene glycol (PEG), PEG-PPG block copolymers, polyoxamer, PEG conjugated fatty chain and PEGilated hydrogenated castor oil.

In certain embodiments the at least one dispersant is Cremophor H40.

In numerous embodiments the at least one lipid component is selected from mineral oils and fatty acid esters.

In further embodiments the mineral oils and fatty acids are selected from liquid or solid mono-, di- and triglycerides and waxes.

In numerous embodiments the amphiphilic solvent is an organic solvent miscible in water or an aqueous media.

In numerous embodiments the amphiphilic solvent is selected from ethyl acetate, ethyl lactate, propylene glycol, ethanol, glycerol, isopropanol, N-methylpyrrolidone, and liquid polyethylene glycol (PEG).

The at least one lipid component in the pre-formulation is a material that is different from the dispersant and from the amphiphilic solvent and is generally selected from components which melt at temperatures below body temperature (below 30-37° C.).

With respect to specific actives, in certain embodiments the devices of the invention can comprise actives selected from cannabinoids, curcumin, apomorphine, amphotericin B, cyclosporine, and rapamycin.

In certain embodiments, at least one active agent comprised in the devices of the invention is apomorphine.

In certain embodiments, the devices can comprise at least one poorly water-insoluble or lipophilic active is at least one cannabinoid having affinity to cannabinoid receptor type 1 (CB 1) or cannabinoid receptor type 2 (CB2).

In further embodiments, the devices can comprise the at least one cannabinoid is selected from the group of cannabidiol (CBD), delta-9-tetrahydrocannabinol (THC), cannabichromene (CBC), cannabichromenic acid (CBCA), cannabichromevarin (CBCV), cannabichromevarinic acid (CBCVA), cannabicyclol (CBL), cannabicyclolic acid (CBLA), cannabicyclovarin (CBLV), cannabidiol monomethylether (CBDM), cannabidiolic acid (CBDA), cannabidiorcol (CBD-C1), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), cannabinodiol (CBND), cannabinodivarin (CBVD), cannabinol (CBN), cannabinol methylether (CBNM), cannabivarin (CBV), cannabitriol (CBT), cannabitriolvarin (CBTV), delta-8-tetrahydrocannabinol (Δ8-THC), delta-8-tetrahydrocannabinolic acid (Δ8-THCA), delta-9-tetrahydrocannabivarin (THCV), delta-9-tetrahydrocannabivarinic acid (THCVA) and cannabicitran (CBT) or any combination thereof.

In still further embodiments the devices can comprise at least one cannabinoid which is comprised in an extract of a cannabis plant or any part thereof.

In certain embodiments the devices can comprise at least one cannabinoid which is THC and/or CBD.

In numerous embodiments the solubility of said at least one poorly water-soluble or lipophilic active in water with neutral pH is lower or equal to 10 mg/ml.

In numerous embodiments said at least one poorly water-soluble or lipophilic active comprised in the pre-formulation is atomized to a particle size in the range between about 1 nm and about 1000 nm, or more specifically in the range between about 1-50 nm, 50-100 nm, 100-150 nm, 150-200 nm, 200-250 nm, 250-300 nm, 300-350 nm, 350-400 nm, 400-450 nm, 450-500 nm, 500-550 nm, 550-600 nm, 600-650 nm, 650-700 nm, 700-750 nm, 750-800 nm, 800-850 nm, 850-900 nm, 900-950 nm and 950-1000 nm.

Devices of the invention can be configured to adhere to a mucosal tissue in the oral cavity. While maintaining intimate contact with the tissue, the devices allow efficient transfer of the active agent present in the PNS to and through the mucosal tissue to the bloodstream (hence systemic delivery) or to the oral cavity.

The device may appear in different forms, wherein in some embodiments, the device can be in a form of a mucoadhesive patch, a tablet, a sticker, or any other form as long as it has at least one exposed surface that can be intimately adhered or associated with an oral tissue. The size and shape of the exposed area can vary to permit maximum contact and efficient absorption. Non-limiting examples are devices with circular, oval or rectangular shape. In some embodiments, the size of the exposed area can be between about 1-2 cm², 2-3 cm² or 3-4 cm².

In numerous embodiments the composition of active(s) on the two sides of the device with the largest surface area are similar.

In other embodiments the composition of active(s) on the two sides of the device with the largest surface area are distinct.

In other words, the devices of the invention can release the same type of active from both sides (symmetric devices) or different types of actives from each side (asymmetric devices).

Double sided mucoadhesive discs of different compositions in each side can be prepared by either adding to the punch bore the preferred powder composition of one side and on top powder of the desired composition for the other side and compression mold. Alternatively, a double compression can be applied where the first layer in molded at a certain pressure and on top of the formed tablet, add the powder of the second layer and compression mold. Three and more layers can be applied. Another possibility is to prepare a tablet of a single composition and spray coat one side with a solution or a dispersion of the second composition.

Thus, the invention further provides a solid fast dissolving device for a controlled delivery of at least one poorly water-soluble or lipophilic active through at least one tissue in the oral cavity, comprising more than one device according to the above.

In numerous embodiments the devices are substantially two dimensional and horizontally stacked or layered on the sides of with the largest surface area.

In case of delivery of large amounts of active or generally in cases where a larger absorption area is desired, more than one device can be applied to avoid use of unnecessarily large device.

It is another objective of the invention to provide a solid fast dissolving dosage form for a controlled delivery of at least one poorly water-soluble or lipophilic active through at least one tissue in the oral cavity, comprising at least one poly(carboxylic acid) and at least one edible alcohol and the at least one poorly water-soluble or lipophilic active comprised in a pre-formulation with at least one dispersant, at least one lipid component and at least one amphiphilic solvent wherein said active is atomized,
the dosage form and the pre-formulation of the poorly water-soluble or lipophilic active(s) are solid and solvent free, and
the dosage form has a preferential solubility in the oral cavity, whereby the pre-formulation of the poorly water-soluble or lipophilic active(s) is released and delivered through the at least one tissue of the oral cavity.

Specific features of the dosage form of the invention have been described and specified above.

From yet another aspect, the invention provides devices and dosage forms as above for use in local or systemic controlled delivery of at least one poorly water-soluble or lipophilic active through at least one tissue in the oral cavity.

In numerous embodiments the devices and dosage forms of the invention are used in treating at least one disorder, or at least one pre-clinical or clinical condition manifested in at least one tissue or organ of the oral cavity. Common nonlimiting examples of such conditions are dental and gum disease and pain associated therewith, dry mouth, mouth ulcers, aphthae, fungal, viral, and bacterial infections, halitosis, sore throat, etc.

In other embodiments the devices and dosage forms of the invention are used in treating at least one disorder, or at least one pre-clinical or clinical condition having a systemic manifestation. Non limiting examples are neurological disorders and pain, cardiovascular disorders, inflammatory conditions, diabetes, systemic viral, and bacterial infections, and cancer.

In numerous embodiments the devices and dosage forms of the invention are adapted for buccal, sublingual administering.

In numerous embodiments the devices and dosage forms of the invention are adapted for adherence to at least one mucosal tissue in the oral cavity or to the dorsal tongue mucosal tissue or to the muscle of the tongue.

In numerous embodiments the devices and dosage forms of the invention are used in treating at least one ulcer or a lesion in the oral cavity.

In such cases, in certain embodiments the devices and dosage forms of the invention are adapted for application or placement on the ulcer(s) or lesion(s) in the oral cavity.

The invention further provides devices and dosage forms for use in treating at least one disorder, or at least one pre-clinical or clinical condition treatable by one or more cannabinoid(s). There are many proposed medical uses of specific cannabinoids (predominantly THC and CBD) and cannabis, the most common include but not limited to post-injury pain, depression, sleep disorders, anorexia, post-traumatic disorder, inflammatory conditions such as psoriasis (CBD in particular), multiple sclerosis (MS), autistic spectrum disorders and epilepsy (certain strains of cannabis), various types of pain (THC in particular) and as antiemetics.

From yet another aspect, the invention provides methods for a local or systemic controlled delivery of at least one poorly water-soluble or lipophilic active through at least one tissue in the oral cavity of a subject, such methods comprise administering to the subject a device or a dosage form of the invention as described above.

From another point of view, the invention provides a series of methods for treating various clinical and pre-clinical conditions in the oral cavity or systemic conditions in subjects in need thereof. Examples both, the oral and the systemic conditions treatable by the devices and dosage forms of the invention have been referred to above.

Specifically, it is objective of the invention to provide methods of treating at least one disorder, or at least one pre-clinical or clinical condition manifested in at least one tissue or organ of the oral cavity of a subject.

It is another objective of the invention to provide methods of treating at one disorder, or at least one pre-clinical or clinical condition having a systemic manifestation in a subject.

A general feature of all these methods is that the methods comprise administering to the subject a device or a dosage form of the invention as described above.

In numerous embodiments the methods of the invention comprise buccal or sublingual administering of the device or the dosage form to the subject.

In further embodiments said administering comprises adherence of the device of the dosage form to at least one mucosal tissue in the oral cavity of the subject, or to the subject’s dorsal tongue mucosal tissue or to the muscle of the tongue.

It is another objective of the invention to provide methods of treating at least one ulcer or a legion in the oral cavity of a subject, such methods comprise administering to the subject a device or a dosage form of the invention.

In numerous embodiments such methods comprise an application or a placement of the device or the dosage form on the ulcer(s) or lesions(s) in the subject’s oral cavity.

It is another objective of the invention to provide methods of treating at least one disorder, or at least one pre-clinical or clinical condition treatable by one or more cannabinoid(s) in a subject, the method comprises administering to the subject a device or a dosage form of the invention. Relevant applications and conditions have been discussed above.

The term ‘about’ herein denotes up to a ±10% deviation from the specified values and/or ranges, more specifically, up to ±1%, ±2%, ±3%, ±4%, ±5%, ±6%, ±7%, ±8%, ±9% or ± 10% deviation therefrom.

### EXAMPLES

Any methods and materials to those described herein can be used in the practice or testing of the present invention. Some embodiments of the invention will be now described by way of examples with reference to respective figures.

### EXAMPLE 1 Compression Molded Devices for Extended Delivery of Agents in the Oral Cavity and the Gastrointestinal System

Compression of poly(acrylic) acid (PAA) and polyol powders usually yields rigid and non-flexible devices, i.e., tablets, stickers, or films. The objective was to obtain flexible devices by a simple process with minimal change in the Carbopol 934 (CP): Hydroxypropylcellulose (HPC) composition. Surprisingly, the inventors have found that the addition of a small amount of alcohol to the composition yields a flexible device, and under certain conditions can even increase the adhesion capacity to the mucosal tissue and tongue of a cow. The following examples demonstrate several features of such devices.

### 1.1 Effect of the Composition and Roughness on the Adhesion Properties

Several types of devices were made by compression molding and placed on the cow’s tongue. Detachment force was determined by tensiometer. The results are shown in Tables 1 and 2.

TABLE 1

Adhesiveness of the devices to the tongue

| CP % | Type of tablet | Detachment force (N) |
|---|---|---|
| 100 | smooth tablet | 1.813 |
| | rough tablet 200 | 2.807 |
| | rough tablet 300 | 6.438 |
| | rough tablet 500 | 8.004 |
| 60 | smooth tablet | 0.224 |
| | rough tablet 200 | 0.252 |
| | rough tablet 300 | 0.324 |
| | rough tablet 500 | 0.4768 |

Devices’ diameter D = 14 mm

TABLE 2

Adhesiveness of the devices to the buccal mucosal tissue

| CP % | Type of tablet | Detachment force (N) |
|---|---|---|
| 100 | smooth tablet | 0.753 |
| | rough tablet 200 | 0.958 |
| | rough tablet 300 | 0.982 |
| | rough tablet 500 | 0.668 |
| 60 | smooth tablet | 0.5366 |
| | rough tablet 200 | 0.6522 |
| | rough tablet 300 | 0.7324 |
| | rough tablet 500 | 0.9477 |

Diameter D = 1.4 cm

Conclusions: Adhesion to the tongue is significantly affected by the composition and roughness of the device surface.

### 1.2 Effect of CP Content and Roughness on the Adhesion Properties

Devices were made of HPC:CP in different ratios - 20%, 40%, 60%, 80%, 100% CP. HPC and CP were gently mixed using a mortar and pestle. Devices (D=14 mm) were pressed by Carver press (Carver Machine Works, Inc., Washington, NC) by pressure of 3 ton/cm² for 10 s.

Devices with smooth and right textures of opposite sides with the larges surface area were tested vs. devices with smooth surfaces (controls). Adhesion to the mucosal tissues, tongue and muscle under the mucosal tissue was tested using tensiometer. The results are shown in Tables 3 to 5.

TABLE 3

Adhesiveness of the devices to the tongue

| CP % | Type of tablet | Detachment force (N) |
|---|---|---|
| 100 | smooth tablet | 1.238 |
| | rough tablet 100 | 1.428 |
| | rough tablet 200 | 2.314 |
| | rough tablet 300 | 3.400 |
| | rough tablet 500 | 6.912 |
| 80 | smooth tablet | 1.093 |
| | rough tablet 100 | 1.164 |
| | rough tablet 200 | 1.828 |
| | rough tablet 300 | 2.273 |
| | rough tablet 500 | 3.125 |
| 60 | smooth tablet | 0.831 |
| | rough tablet 100 | 0.962 |
| | rough tablet 200 | 1.036 |
| | rough tablet 300 | 1.047 |
| | rough tablet 500 | 1.285 |
| 40 | smooth tablet | 0.611 |
| | rough tablet 100 | 0.771 |
| | rough tablet 200 | 0.932 |
| | rough tablet 300 | 0.994 |
| | rough tablet 500 | 1.039 |
| 20 | smooth tablet | 0.338 |
| | rough tablet 100 | 0.510 |
| | rough tablet 200 | 0.611 |
| | rough tablet 300 | 0.749 |
| | rough tablet 500 | 0.994 |

TABLE 4

Adhesiveness of the devices to the mucosal tissue

| CP % | Type of tablet | Detachment force (N) |
|---|---|---|
| 100 | smooth tablet | 1.138 |
| | rough tablet 100 | 1.809 |
| | rough tablet 200 | 2.323 |
| | rough tablet 300 | 2.705 |
| | rough tablet 500 | 3.902 |
| 80 | smooth tablet | 1.033 |
| | rough tablet 100 | 1.605 |
| | rough tablet 200 | 1.949 |
| | rough tablet 300 | 2.335 |
| | rough tablet 500 | 3.005 |
| 60 | smooth tablet | 0.931 |
| | rough tablet 100 | 0.942 |
| | rough tablet 200 | 1.010 |
| | rough tablet 300 | 1.749 |
| | rough tablet 500 | 2.045 |
| 40 | smooth tablet | 0.701 |
| | rough tablet 100 | 0.771 |
| | rough tablet 200 | 0.982 |
| | rough tablet 300 | 1.099 |
| | rough tablet 500 | 1.939 |
| 20 | smooth tablet | 0.323 |
| | rough tablet 100 | 0.501 |
| | rough tablet 200 | 0.731 |
| | rough tablet 300 | 0.798 |
| | rough tablet 500 | 1.009 |

TABLE 5

Adhesiveness of the devices to the muscles

| CP % | Type of tablet | Detachment force (N) |
|---|---|---|
| 100 | smooth tablet | 1.138 |
| | rough tablet 100 | 1.809 |
| | rough tablet 200 | 2.323 |
| | rough tablet 300 | 2.705 |
| | rough tablet 500 | 3.902 |
| 80 | smooth tablet | 1.033 |
| | rough tablet 100 | 1.605 |
| | rough tablet 200 | 1.949 |
| | rough tablet 300 | 2.335 |
| | rough tablet 500 | 3.005 |
| 60 | smooth tablet | 0.931 |
| | rough tablet 100 | 0.942 |
| | rough tablet 200 | 1.010 |
| | rough tablet 300 | 1.749 |
| | rough tablet 500 | 2.045 |
| 40 | smooth tablet | 0.701 |
| | rough tablet 100 | 0.771 |
| | rough tablet 200 | 0.982 |
| | rough tablet 300 | 1.099 |
| | rough tablet 500 | 1.939 |
| 20 | smooth tablet | 0.323 |
| | rough tablet 100 | 0.501 |
| | rough tablet 200 | 0.731 |
| | rough tablet 300 | 0.798 |
| | rough tablet 500 | 1.009 |

Conclusions: The devices had superior adherence to all types of tissues, and especially the tongue. The detachment force increased with an increased CP percentage. The adhesiveness of the devices increased with an increased roughness and CP content.

### 1.3 Effect of Glycerol of the Flexibility of the Devices

Devices were made of HPMC:CP (1:4 w/w), 0%, 5% and 10% of glycerol and ethanol. The devices with smooth and rough surfaces on opposite sides with the largest surface area were produced using compression mold uisng upper smooth disc and lower rough disc to obtain roughness of 100, 200, 300 and 500 µm.

Devices containing 10% glycerol had a superior flexibility with bending capacity over 90° compared to 5% glycerol with bending capacity over 45° and 0% glycerol which were rigid and had tendency to break with bending over 10°. Adhesion properties the tongue and mouth mucosal tissues were tested by tensiometer. The results are shown in Tables 6 and 7.

TABLE 6

Adhesiveness of the devices to the tongue

| Glycerol % | Type of tablet | Detachment force (N) |
|---|---|---|
| 0 | smooth tablet | 1.238 |
| | rough tablet 100 | 1.428 |
| | rough tablet 200 | 2.314 |
| | rough tablet 300 | 3.400 |
| | rough tablet 500 | 6.912 |
| 5 | smooth tablet | 1.112 |
| | rough tablet 100 | 1.190 |
| | rough tablet 200 | 2.001 |
| | rough tablet 300 | 2.834 |
| | rough tablet 500 | 4.700 |
| 10 | smooth tablet | 1.005 |
| | rough tablet 100 | 1.097 |
| | rough tablet 200 | 1.117 |
| | rough tablet 300 | 1.993 |
| | rough tablet 500 | 2.801 |

TABLE 7

Adhesiveness of the devices to the mucosal tissue

| Glycerol % | Type of tablet | Detachment force (N) |
|---|---|---|
| 0 | smooth tablet | 1.138 |
| | rough tablet 100 | 1.809 |
| | rough tablet 200 | 2.323 |
| | rough tablet 300 | 2.705 |
| | rough tablet 500 | 3.902 |
| 5 | smooth tablet | 1.002 |
| | rough tablet 100 | 1.305 |
| | rough tablet 200 | 1.798 |
| | rough tablet 300 | 2.001 |
| | rough tablet 500 | 2.704 |
| 10 | smooth tablet | 0.862 |
| | rough tablet 100 | 1.010 |
| | rough tablet 200 | 1.221 |
| | rough tablet 300 | 1.773 |
| | rough tablet 500 | 2.004 |

Conclusions: 10% of glycerol yielded devices with superior flexibility and translucence, but inferior adhesion properties. The devices with 5% glycerol were less flexible but had stronger adhesion.

Flexible two-sided adhesive devices were prepared by layering (three layers) as follows: 20 to 50 mg of CP or HPMC:CP mixture was loaded and spread evenly to cover the lower disc surface; 100 mg HPMC:CP powder containing 10% w/w glycerol was spread on top of this layer; another layer of adhesive powder 20 to 50 mg CP or HPMC:CP mixture without glycerol was added; the powder was compressed into a tablet.

One-sided devices were prepared as follows: the top layer was replaced with polyvinyl pyrrolidone (PVP) or another non-adhesive powder (e.g., microcrystalline cellulose, Eudragit L, starch, ethyl cellulose, etc.) to form a non-adhesive side. Alternatively, no upper layer was used since glycerol reduces the adhesion. FIG. 1 shows a schematic illustration of the multilayer tablet.

The adhesiveness of the devices was tested on tongue and mucosal tissue by using a tensiometer. The results are shown in Table 8.

TABLE 8

Adhesiveness of the devices to the tongue and mucosal tissue

| | Type of tablet | Detachment force (N) of multilayer flexible tablets | Detachment force (N) of one-layer flexible tablets |
|---|---|---|---|
| Tongue | smooth tablet | 1.205 | 1.005 |
| | rough tablet 100 | 1.397 | 1.097 |
| | rough tablet 200 | 2.117 | 1.117 |
| | rough tablet 300 | 2.355 | 1.993 |
| | rough tablet 500 | 3.980 | 2.801 |
| Mucosal tissue | smooth tablet | 1.012 | 0.862 |
| | rough tablet 100 | 1.490 | 1.010 |
| | rough tablet 200 | 2.101 | 1.221 |
| | rough tablet 300 | 2.734 | 1.773 |
| | rough tablet 500 | 3.609 | 2.004 |

Conclusions: Multilayer flexible devices have more superior adhesiveness than one-layer devices. The one-layer devices are slightly more flexible. Ethanol provided less flexibility to the devices.

### 1.4 Mucoadhesive Coated Microparticles

Particles of inert polymers, drug particles or drug loaded particles with a size in the range of 20-200 µm were coated with a mucoadhesive top layer by the following methods:

1.4.1 An alcoholic fine dispersion containing 1.0% w/w of hydroxypropyl cellulose (HPC) and soluble or crosslinked poly(methacrylic acid) (CP 940) at a weight ratio of 1:9 respectively, was sprayed onto pan-coating containing the powder at an amount of 1, 10 and 20% per particles weight (w/w). The coating material was spayed slowly onto the drug powder for 10, 20 and 60 min, while applying warm air to evaporate the alcohol and allow fast coating of the particles. The mixing rate, spraying and drying procedures were adjusted to allow uniform thin coating. Coating amount per particles was modulated by increase in weight of the starting powder. The coating was visualized by swelling the particle with water containing a dye, e.g., methyl orange, phenolphthalaine and food colorants. The thickness of coating was further modulated by the concentration of polymers in the coating solution, increasing the concentration to 2% provided a thicker coating film. Too viscous solution, however, should be avoided as it results in aggregation of the particles into clusters and granular material. Modulation of the ratio of HPC:CP from about 20% to 95% CP further affected coating properties, and the adhesion capacity. Polyvinyl pyrrolidone or polyvinyl alcohol were added to improve coating uniformity and adhesion of the coating to particles. Crosslinked poly(methacrylic acid) and other carboxylic acid containing polymers were used, including carboxy methyl cellulose, cellulose succinate or glycolate as an alternative to CP.

1.4.2 The dispersion of drug particles and mucoadhesive polymer was evaporated to dryness and the formed cake was ground to form particles of proper size, which was farther refined by sieving through a sieve net to remove larger particles. Coating of particles was modulated by dispersing the particles in 1% alcoholic polymer dispersion for few min; the insoluble solids were isolated from the dispersion by decantation of the extra polymer dispersion and drying the wet powder in a fluidized bed to form coated microparticles. The amount of coating was less controlled compared to the pan-coating. The method however was reproducible under the same coating conditions. It should be noted that it is not essential to fully cover each particle with the mucoadhesive coating, partial coating can be sufficient for adherence to the desired site. This coating process is limited to drugs that are insoluble or hardly soluble in alcohol, so the drug does not dissolve during the coating process. To overcome this limitation, the drug or inert powder was coated first with a thin layer of water-soluble polysaccharide such as dextran or polyvinyl alcohol to provide a hydrophilic protection prior to the coating with the mucoadhesive polymer as above.

### 1.5 Buccal Delivery of Cannabinoids, THC and CBD

Devices containing cannabinoids THC and/or CBD were prepared by: (1) compression of a mixture of cannabinoid agents and mucoadhesive polymers such as hydroxypropyl cellulose (HPC) and crosslinked poly(methacrylic acid) (CP); or (2) a prior formulation of the cannabinoids into a lipid based formulation, Lipospheres; this preparation further provides protection of the active agents and prevents them from being metabolized when crossing the tissue to the systemic circulation and further assists in solubilizing these hydrophobic molecules to allow better absorption.

A typical device contained 70-95% of mucoadhesive polymers HPC and CP at 1:3 w/w and liposphere pre-formulation with 5-20% cannabinoids. Alternatively, a device can be produced of the mucoadhesive polymers where a cavity is formed in the center of the adhesion side for loading the liposphere formulation of CBD and THC, while the cavity is configured to provide a direct contact with the tissue for better absorption.

A typical formulation of 200 mg sticker is composed of HPC 40 mg and CP 940-90 mg as sticker matrix, and liposphere CBD formulation composed of Tween 20-15 mg, Span 80-15 mg, tricaprin-15 mg, CBD 10 mg and phospholipid 15 mg. The liposphere components were melt at 60° C. to form a uniform oily liquid, mixed with 50% of the matrix powder, the powder was mixed with the other 50% of matrix powder, and compressed into a sticker with pressure of 2 ton/cm². One-side stickers were covered with either a thin layer of wax or HPC to block the release of active agents to the oral cavity. Alternatively, stickers with cavities on the adhesion side were loaded with the liposphere formulation covered with a thin protecting layer that was removed upon use.

Both stickers adhered well the buccal mucosa and released CBD for 4 h, after which the stickers started to erode.

### 1.6 Flexible Bio-Adhesive Devices

Bio-adhesive devices containing 20% HPC, 80% of CP 934 (CP) and 0%, 5%, 10% and 20% glycerol were prepared. HPC and CP powders were gently mixed using a mortar and pestle. Devices (D=14 mm) were pressed by Carver at 3 ton/cm² for 10 sec, using molds with one smooth and one rough surfaces.

Devices with two different surfaces, smooth and the rough layers, were compared to two-sided smooth devices (control). The devices were placed on the cow tongue and kept in moisture room for 2 h, 4 h, and 6 h. Adhesion was tested by using tensiometer. The results on the hard and flexible devices and different incubation times are shown in Tables 9 and 10.

TABLE 9

Adhesiveness of the hard devices to the tongue

| Time (hours) | Type of tablet | Detachment force (N) |
|---|---|---|
| 0 | smooth tablet | 2.714 |
| | rough tablet 100 | 3.356 |
| | rough tablet 200 | 3.386 |
| | rough tablet 300 | 4.289 |
| | rough tablet 500 | 5.933 |
| 2 | smooth tablet | 1.871 |
| | rough tablet 100 | 2.665 |
| | rough tablet 200 | 2.636 |
| | rough tablet 300 | 3.225 |
| | rough tablet 500 | 5.288 |
| 4 | smooth tablet | 1.229 |
| | rough tablet 100 | 1.805 |
| | rough tablet 200 | 2.036 |
| | rough tablet 300 | 2.915 |
| | rough tablet 500 | 4.081 |
| 6 | smooth tablet | 0.929 |
| | rough tablet 100 | 0.850 |
| | rough tablet 200 | 1.719 |
| | rough tablet 300 | 1.952 |
| | rough tablet 500 | 3.095 |

Conclusions: After 6 h in moisture room the adhesiveness decreases but remains relatively good. Rough tablets adhere better than the smooth tablets.

TABLE 10

Adhesiveness of the flexible devices to the tongue

| Glycerol (%) | Type of tablet | Detachment force after 0 h in moisture room | Detachment force after 2 h in moisture room |
|---|---|---|---|
| 5 | smooth tablet | 1.312 | 1.112 |
| | rough tablet 100 | 1.965 | 1.799 |
| | rough tablet 200 | 2.001 | 1.911 |
| | rough tablet 300 | 3.834 | 2.734 |
| | rough tablet 500 | 4.99 | 4.7 |
| 10 | smooth tablet | 1.005 | 0.997 |
| | rough tablet 100 | 1.697 | 1.6 |
| | rough tablet 200 | 1.907 | 1.899 |
| | rough tablet 300 | 2.993 | 2.704 |
| | rough tablet 500 | 4.801 | 4.797 |
| 20 | smooth tablet | 0.994 | 0.669 |
| | rough tablet 100 | 1.008 | 0.732 |
| | rough tablet 200 | 1.501 | 1.19 |
| | rough tablet 300 | 2.25 | 1.992 |
| | rough tablet 500 | 4.232 | 3.832 |

Conclusions: Detachment force is reciprocally correlated to higher content of glycerol. For example, the detachment force for 20% glycerol rough tablet 500 was higher than for 5% glycerol rough tablet 500. Adhesiveness decreases with time but remains relatively good. In general, the devices have superior adherence to the tongue, and are difficult to detach even after 6 h in water.

Devices containing 5% of sodium carbonate and lactic acid were prepared. When saliva is absorbed in the device, it will react with Na₂CO₃ to produce CO₂ which could help to detach it from the tongue. The devices were put on 5% lactic acid solution for 1 min and tested by Tensiometer. The results are shown in Table 11.

TABLE 11

Effect of sodium carbonate and lactic acid

| | Sodium carbonate (%) | Type of tablet | Detachment force (N) |
|---|---|---|---|
| 0 min in lactic acid 6 h in water | 0 | smooth tablet | 0.929 |
| | | rough tablet 100 | 0.85 |
| | | rough tablet 200 | 1.719 |
| | | rough tablet 300 | 1.952 |
| | | rough tablet 500 | 3.095 |
| 1 min in lactic acid 6 h in water | 0 | smooth tablet | 0.511 |
| | | rough tablet 100 | 0.599 |
| | | rough tablet 200 | 1.161 |
| | | rough tablet 300 | 1.585 |
| | | rough tablet 500 | 2.005 |
| 0 min in lactic acid 6 h in water | 5 | smooth tablet | 0.889 |
| | | rough tablet 100 | 1.085 |
| | | rough tablet 200 | 1.432 |
| | | rough tablet 300 | 2.129 |
| | | rough tablet 500 | 3.035 |
| 1 min in lactic acid 6 h in water | 5 | smooth tablet | 0.311 |
| | | rough tablet 100 | 0.423 |
| | | rough tablet 200 | 0.688 |
| | | rough tablet 300 | 1.052 |
| | | rough tablet 500 | 1.673 |

Conclusions: Generally, devices containing 80% CP adhere very well to the tongue even after 6 h on moisture. Reaction between lactic acid and Na₂CO₃ decreases the adhesion. While lactic acid decreased the adhesion, devices containing 5% Na₂CO₃, detached smoothly.

### 1.7 Ring Bio-Adhesive Devices Releasing to the Center

Special devices were prepared for treating oral lesions such as aphtha. A device (tablet) in a form of a ring where the inner side is void was prepared by compression molding of the powder into a mold having a disc in the center of the lower or upper side of the punch surface. The diameter and height of the centered disc is dependent on the desired device, the inner diameter of the void of the ring and the thickness of the ring. The ring can be solid on one side without a hole when applying powder that is above the height centered disc, so that during compressing the powder a full cover is obtained on one side. This kind of tablets are particularly useful for treating oral lesions or ulcers that should remain free of adhesive cover by isolated from the oral environment and delivers the active agents towards the center where the ulcer is located.

Another application of such ring design is for the delivery agents that are placed within the cavity without adhesion capability. Tablet-ring was loaded with 0.1, 1, 2, and 5 10% (w/w) of chlorhecidine gluconate antimicrobial agent or iodine complex on HPC or CP and were released from the ring after adhering to mucosal tissue or tongue in the oral cavity.

### 1.8 Delivery of Biologically Active Agents by Mucoadhesive Devices

Peptides or proteins stabilized or encapsulated into nanoparticles, microparticles or liposomes were mixed in CP and HPC as described above and compressed into tablets. In a typical example, insulin zinc salt powder at 1% of the solids was added to the tablet composition and compressed into tablets. Insulin was released for 10 h from the tablets when placed in physiologic media. PLGA microspheres loaded with 50% insulin prepared by precipitation of concentrated alcoholic-dichloromethane solution into an anti-solvent, heptane. Particles at 10% of the non-active agents were added to the tablet powder and compressed into tablets. Anti-inflammatory protein such as Adiponectin, antimicrobial and antiviral proteins are also considered for buccal delivery.

Additional examples of biologically active agents include, but not limited to probiotic bacteria such as Lactobacillus acidophilus, Bifidobacterium spp., and Lactobacillus casei and further, biological molecules such as siRNA and plasmid DNA. These types of actives can be provided in a powder form and incorporated into bio-adhesive devices to provide controlled release of the actives in the oral cavity, buccal absorption, and systemic release. They can be incorporated into different designs and different parts of the devices, such as into the bio-adhesive layer, placed on top of the bio-adhesive layer to provide release of actives to the oral cavity, or placed in the center of the device so that the adhesion in only in the ring.

### 1.9 Bio-Adhesive Devices for Treating Xerostomia

Powders of CP 934 and HPC at a 4:1 w/w ration containing 10% glycerol were mixed with one or more xerostomia therapeutic agents. Candidate agents can include, but not limited to, chondroitin sulfate, coenzyme Q, spirooxathiolane-quinone, polycarbophil, oral antiseptics and oral mucosal protective agents, zinc oxide . Sialogogic agents can include pilocarpine, cevimeline, anethole trithione, yohimbine, human interferon alpha and amifostine. Pilocarpine is a cholinergic parasympathomimetic agent, which can stimulate salivary flow and produce clinical benefits in some patients but can cause adverse effects with other drugs. Cevimeline, a cholinergic agonist, is a systemic agent that can alleviate xerostomia in some patient. Anethole trithione is a cholagogue that stimulates salivary flow in drug-induced xerostomia. Yolimbine is an alpha-2 adrenergic antagonist that can increase saliva flow. Sialogogic agents can further include biological agents, such as probiotic bacteria of various species of the genera Bifidobacterium and Lactobacillus.

The agent is to be included in a therapeutically safe amount and dose, and preferentially released at the site of salivary glands. The device should contain sufficient doses of the agent such that the agent is released for the desired time period at an effective amount to treat or alleviate symptoms of xerostomia. The effective amount is typical for each agent. The desired release profile can be determined by standard methods by one of ordinary skill in the art. Typical doses for biological agents, such as probiotic bacteria, range from about 0.1 µg to few µg in a device (tablet or film), depending on the activity of the dry substance and the desired effect.

### 1.10 Bio-Adhesive Releasing Homeopathies Agents

Additional type of mucoadhesive devices was contemplated for the treatment of dermatitis using a low dose, homeopathic medication such as potassium bromide, sodium bromide, nickel sulfate, and sodium chloride. The actives were incorporated in HPC:CP containing 5% glycerol and compressed into a device (tablet) to provide controlled release for 6 h.

### 1.11 Asymmetric Devices

Asymmetric devices releasing different active agents from each side were prepared by compression molding of two powders containing two different agents, on one side an agent to be released within the oral cavity and on the other side another agent to be delivered across the buccal tissue for local or systemic effects. This type of devices can release different agents to each side of the adherent tissue surface, when it adheres to the tongue and the buccal tissue, for example. In other terms, these are double-sided devices releasing different agents to each side of the adherent tissue or environment.

Tablets made from compression molding a mixture of two layered powders of CP and HPC containing lidocaine on one side and chlorhexidine gluconate on the other side. An intermediate layer made either from the same composition without any active agents or an inert powder such as ethyl cellulose, HPC or fatty acids was used to minimize cross-delivery of agents.

### 1.12 Effect of Glycerol on the Adhesion and Flexibility of CGN Tablets

A stock mixture of Carbopol71G NF (CP) 80% (w/w), hydroxypropyl cellulose (HPC) 10% (w/w), k-carrageenan (CGN) 10% (w/w) and magnesium stearate 0.1% (w/w) was prepared. Glycerol, CGN and HPC were mixed then CP was added and mixed (see Table 12). Devices (tablets D=13 mm) were prepared by compression molding loading 200 mg of the final mixture into flat plate discs under pressure of 2 tons.

TABLE 12

Concentration of glycerol in the formulation

| | Carbopol 71G NF [%] | HPC [%] | k-carrageenan CGN [%] | Glycerol [%] |
|---|---|---|---|---|
| Blank | 80 | 10 | 10 | 0 |
| 5% glycerol | 76 | 9.5 | 9.5 | 5 |
| 7% glycerol | 74.4 | 9.3 | 9.3 | 7 |
| 10% glycerol | 72 | 9 | 9 | 10 |

Self-alignment mechanism was used to measure net adhesion force and to prevent sliding of the tablet. Each formulation was attached to the glass slide using ethyl cyanoacrylate adhesive then fixed the slide to upper arm into slide house with bolts, also another flat glass slide fixed to the lower substrate using bolts and (2.6x3.8 cm).

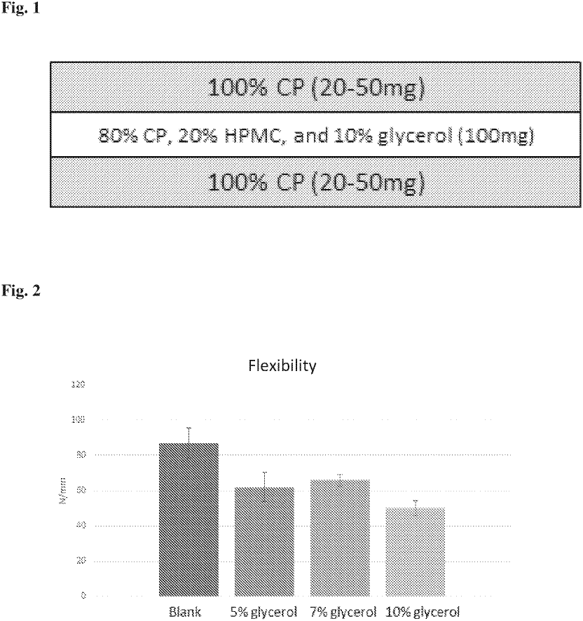

The effect of glycerol on the flexibility of the tablets is shown in FIG. 2. The hardness of the tablets slightly increased with the increase of glycerol content. The increase in hardness was in the range of 5-12% relative the tablet without glycerol. Further, friability was tested in fifteen tablets put in the container for 4 min at 25 RPM. The results are shown in Table 13.

TABLE 13

Effect of glycerol on friability of the devices.

| | Weight before[g] | Weight after[g] | Rate |
|---|---|---|---|
| Blank | 2.978 | 2.983 | 1.00 |
| 5% glycerol | 3.007 | 3.004 | 1.00 |
| 7% glycerol | 2.999 | 3.004 | 1.00 |
| 10% glycerol | 3.006 | 3.009 | 1.00 |

Effect of compression molding on mechanical properties: A stock mixture of CP 80% (w/w), HPC 10% (w/w), CGN 10% (w/w) and Magnesium stearate 0.1% (w/w) was used in combination with 2%(w/w) lemon oil. The first set of formulations was without glycerol and the second set was with 7% (w/w) glycerol. Mixtures were prepared by mixing glycerol, lemon oil, CGN and HPC, and then added CP (see Table 14). Tablets (D=13 mm) were prepared by compression loading 200 mg powder mixture into flat plate discs under pressure of 1, 3, 5 and 7 tons.

TABLE 14

Concentration of ingredients in the formulation.

| Carbopol 71G NF [%] | HPC [%] | CGN [%] | Glycerol [%] | Lemon oil [%] |
|---|---|---|---|---|
| 78.4 | 9.8 | 9.8 | 0 | 2 |
| 72.8 | 9.1 | 9.1 | 7 | 2 |

To determine the adhesion to glass, tablets were attached to flat glass surface and pulled by tensiometer. One side of the tablet was fixed with cyanoacrylate glue. The top glass slide was wetted by buffer solution (50 µl of Phosphate buffer, pH 6.8) and the tablet was lowered (0.1 mm/s) until normal load of 5 N was obtained to allow adherence of the tablet to the upper glass side. To measure the maximum adhesion force, the machine pulled the upper glass at the constant rate of 5 mm/min, the force was recorded until detachment. Adhesion force was determined as the maximal force to detach the tablet as an average of five independent measurements. The experiment was performed at room temperature. The compression force had little effect on the adhesion properties.

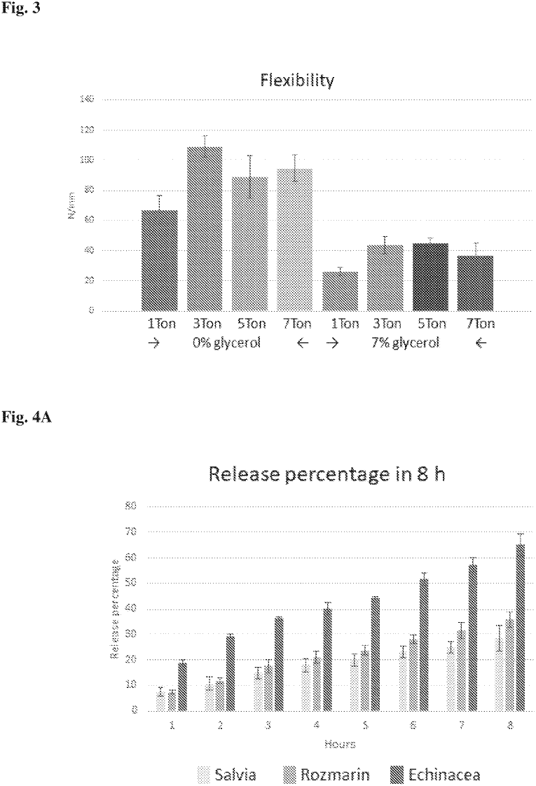

To measure the flexibility of tablet, a test device was developed where the tablet was placed on a ring of the same dimeter as the tablet and an upper arm of a 4 mm ball shape was placed onto the surface of the tablet. To measure the flexibility, the ball arm was forced down at a certain rate and bended the tablet until it broke. The slope of the linear stage of the deformation vs. deformation rate was determined. The results are shown in FIG. 3.

Conclusions: Tablets containing 7% glycerol were significantly more flexible than non-glycerol containing tablets, regardless of the compression. Similar results were obtained when 10% ethanol or propylene glycol were used as small molecule alcohols. The thickness of the tablet was not significantly affected by the compression force. All tablets were in the range of 1.29-1.58 mm thickness.

### 1.13 Devices Loaded With Plant Extracts

Tablets contained CP 971P, HPC, CGN, magnesium stearate and plant extract (Echinacea, Saliva and Rosmarinus). A stock mixture of CP 80% (w/w), HPC 10% (w/w), CGN 10% (w/w) and Magnesium stearate 0.1% (w/w) were mixed with extracts of Echinacea, Saliva or Rozmarin (see Table 15). Tablets (D=13 mm) were prepared as above under pressure of 2 MPa using compression molding device.

TABLE 15

Concentration of ingredients in the formulations

| Formulation | Dyclonine | Mannitol | Stock mixture |
|---|---|---|---|
| Blank | - | - | 100% |
| Echinacea | 1 % | - | 99% |
| Saliva | 1 % | - | 99% |
| Rosmarinus | 1 % | - | 99% |

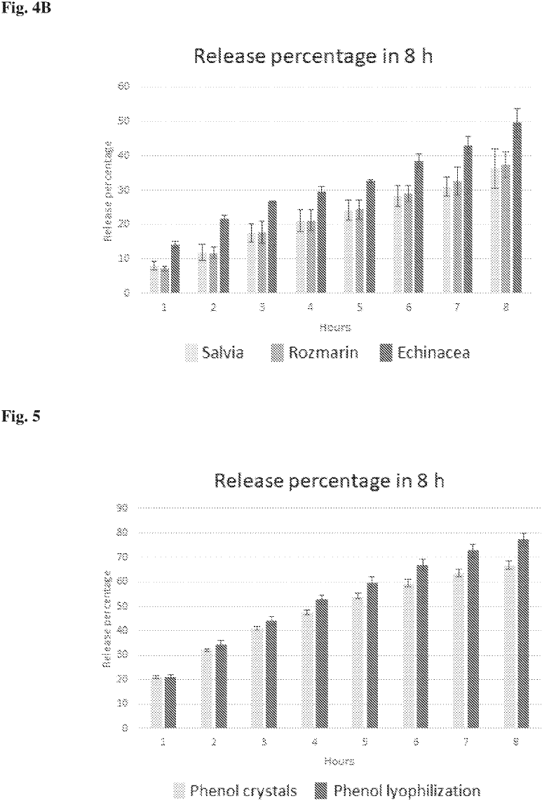

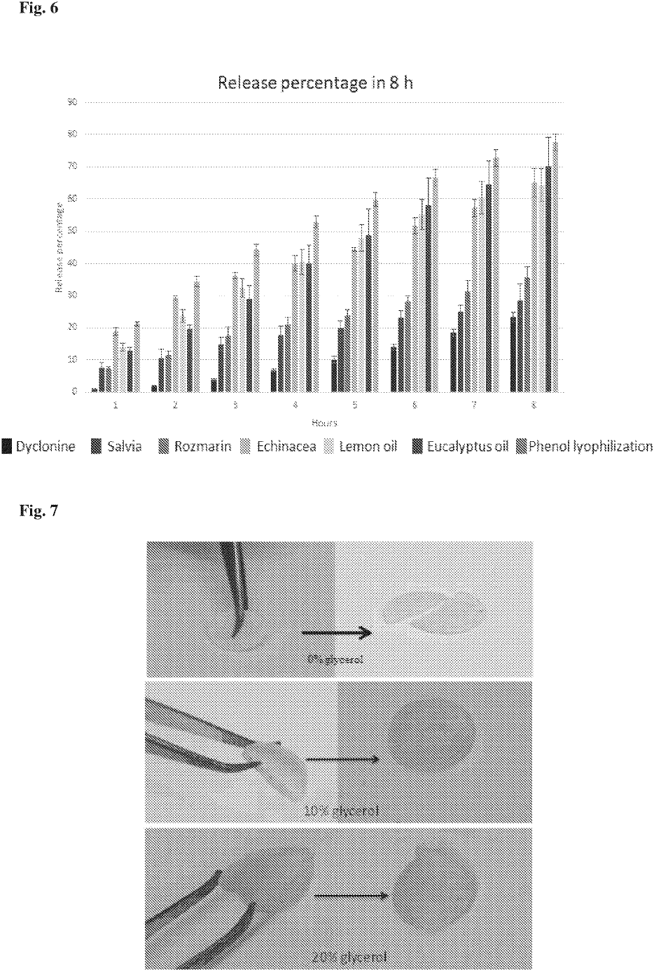

Tablets were fixed to the bottom of 20 ml scintillation vials 4 ml phosphate buffer 6.8 pH was added to the vials at 37° C., the solution was changed every hour for 8 h and saved for content determination at 280 nm and 320 nm for plant extracts. Measurements were performed in triplicates. The release results are shown in FIGS. 4A-4B. The effect of lyophilization on phenol crystals in the release study is shown in FIG. 5 and FIG. 6.

Conclusions: Overall, the results suggest that the active ingredients are constantly released from the adhesive tablets. Better results by 10% increased release were obtained after phenol lyophilization.

### 1.14 Application in Human Subjects

Adhesive devices composed of 91% adhesive carrier were made from the mixture of CP971, HPC an sulfated CGN at 8:1:1 (w/w), 7% w/w glycerol, 1% w/w dyclonine and 1% w/w menthol used in a healthy volunteer. The 200 mg tablets (D=14 mm, h ~1.5 mm) were placed on saliva-free buccal and pushed for 10 sec to guarantee full surface adhesion to the buccal tissue surface. The tablet remained in the mouth for about 4 h until fully eroded. During 4 h, the subject reported no limitation in talking, drinking, and eating. The flat, smooth and flexible released mint flavor throughout the time of erosion.

### 1.15 Mucoadhesive Lozenges for Treating Oral Infections and Sore Throat

Mucoadhesive lozenges were developed to provide agents release to the oral cavity for reducing bacterial infection, inflammation and pain, and improve healing for treating microbial or viral infections with inflammation related oral disorders such as sore throat, oral aphtha’s ulcers, gingivitis and halitosis. Oral infection and inflammation can affect all tissues of the oral cavity, e.g., the gingiva, the tongue, upper respiratory system, and teeth, in different usually tissue specific manners. Treatment of the oral cavity with agents that reduce pathogenic bacteria and viruses as well as inflammation symptoms, can help patients overcome these conditions. Sore throat or pharyngitis can be caused by microbial agents such as Mycoplasma pneumoniae, Streptococcus, Neisseria gonorrhoeae and Chlamydia pneumonia or by environmental contaminants and other non-microbial factors. These conditions are more common in the winter.

Throat lozenges can contain local anesthetics such as lidocaine, benzocaine and natural ingredients such as eucalyptus oil. Additional candidate actives are menthol, pectin or zinc gluconate, phenolic derivatives and dextromethorphan. Various brands of throat lozenges include Cepacol, Butter-Menthol, Chloraseptic, Gorpils, Fisherman’s Friend, Halls, Lockets, Läkerol, Pastilles Juanola, Luden’s, Ricola, Robitussin, Strepsils and Smith Brothers. Some other brands are Tunes, Vigroids, Vicks, Victory V, Sucrets and CVS Throat Drops.

Smooth and rough tablets contained 80% CP and 20% HPC were prepared and further tablets with 80% CP and 20% HPMC and 5%, 10% and 20% glycerol. As previously shown, the addition of glycerol leads to increase in tablet flexibility. FIG. 7 illustrates the difference between hard and flexible tablets.

Adhesiveness of tablets was evaluated on the mucosal tissue (substrate) using pull-off adhesive tests by tensiometer. Tablet were contacted with the mucosal tissue under a given normal load, then separated while measuring the adhesive force. Essentially, the adhesion force was similar up to 10% glycerol content and a higher glycerol content reduced the adhesion properties.

Buccal drug delivery in oral cavity: Mucoadhesive tablets were prepared by compression molding using 200 mg mixture of HPC:CP 934 2:8, glycerol 5% and 12 mg of one of the following drugs: benzocaine (local anesthetic), amphotericin B (Amp B, antifungal drug), chlorhexidine (topical antimicrobial) and nicotine. Herbal extracts of Curcuma longa (Turmeric) and Foeniculum Vulgare were also incorporated.

The release test was done in phosphate buffer pH=6.8 at 37° C. for 6 h. The release of actives was measured as above using UV spectroscopy. Tablets were attached to the bottom of 20 ml vials (tablet per vial), 5 ml phosphate buffer (pH= 6.8) was added, and vials were incubated at 37° C. with constant shaking of 75 rpm. The active agents were constantly released from the tablets over the period of 6 h. Curcuma and Foeniculum extracts showed zero order release profile with over 90% release of the agents. The tablets eroded within the period parallel to the release of the loaded active agents.

### 1.16 Gastro-Retentive Compression Molded Flexible Films

Films were prepared by compression molding of acrylic acid based polymers such as Eudragit S, E and L, and Carbopol; HPC and HPMC, and ethylene glycol polymers such as polyethylene glycol (PEG) 400-35000 MW, block copolymers of ethylene and propylene glycol such as Polyoxamer or Pluronic, glycerol and food oils and waxes. In certain formulations, linear or crosslinked polyvinylpyrrolidone, gelatin powders, ethyl cellulose and pH sensitive polymers such as cellulose acetate phthalate or glyconate were incorporated.

An important prerequisite is that the films should be flexible so they can be folded into a swellable size or packed into a capsule. Upon oral intake, the film is unfolded into a sheet that can be retained in the stomach for at least one hour before degrading and passing to the intestine.

The films were prepared by compression molding of blends of powders containing 1 to 45% active agent(s) under pressure ranging from 1-10 tons per cm². Various active agents such as antibiotics, anti-inflammatory agents, antifungal and antiviral agents, L-Dopa and others were incorporated into formulations by compression molding. Generally, 10% glycerol, 45% HPC and 45% Eudragit S100 were mixed, 100 mg uniform powder was compression molded at 3 tons/cm² into 150 µm film (D=2.5 cm). The films were flexible allowing insertion into a hard gelatin capsule. After 7 min in water, the film completely unfolded on the surface. Further, a mixture of L-Dopa powder 40% (w/w) was mixed with HPC 40%, ethyl cellulose 10% (w/w) and 10% glycerol. The powder was molded into a round flexible partly transparent film of 2.5 cm. The film remained intact in water for at least 5 h. In acidic water solution simulating the stomach fluids, the release of L-Dopa lasted for a few hours.

### 1.17 Fast Dissolving Compression Molded Films

Fast dissolving films are usually placed onto the tongue for the immediate delivery of actives and anti-malodor agents such as menthol, lemon and eucalyptus extracts. Such films can be made from highly water-soluble polymers such as gelatin, polyvinyl-pyrrolidone, ethylene glycol etc., (see for example Arun Arya et al, Int.J. Chem Tech Res 2010 2(1):576-586). These films are prepared in large scale using solvents and heat for curing. This process is labor intensive, require specialty processing machinery and environmental conditions of moisture and temperature. Further, the use of solvent such as water, alcohols or acetone require certain equipment and conditions for uniform evaporation to form uniform, air-bubble free, thin films. The requirement of moisture and temperature limits the active agents to those that are stable under these conditions and non-volatile to escape the film during fabrication.

The objective was to provide a simple and economic fabrication process that is not limited by specific agents. The proposed process of compression molding of powders containing glycerol and other polyols can provide flexible almost transparent films that can be easily adapted to achieve the required solubilization rate - immediate solubilization 30 min or 60 min after application on the tongue or other tissues in the oral cavity.

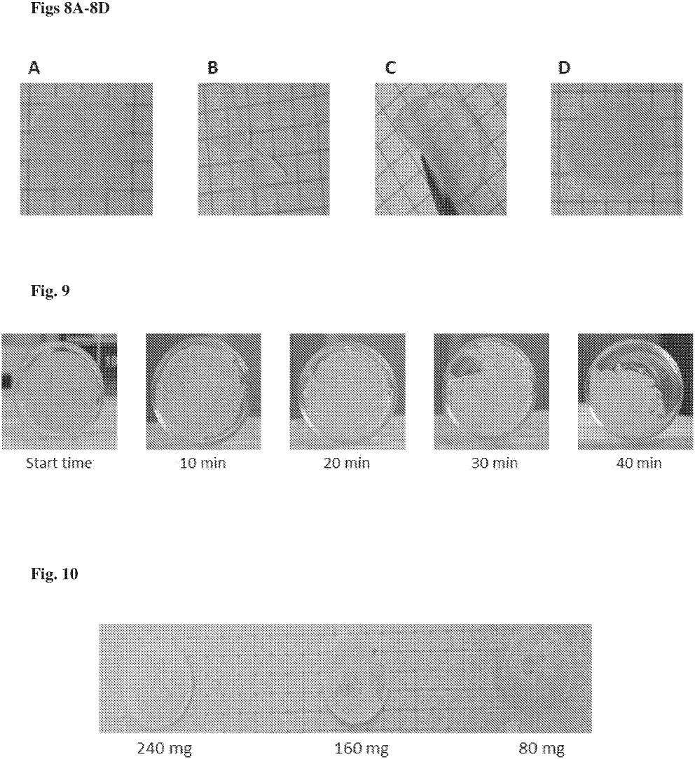

In this experiment, polyvinylpyrrolidone, poly(vinyl alcohol), poly(acrylic acid) PAA, and hydroxypropyl cellulose were used to make flexible films. Film comprising 90% (w/w) polymer mixture and 10% (w/w) glycerol were prepared. 100 mg of the mixture was loaded on a compression barrel of 2.4 cm round mold under 10 tons force. The results are shown in Table 16 and FIGS. 8A-8D.

TABLE 16

Flexibility and transparency of the films with various polymers

| | | | |
|---|---|---|---|
| Composition | Flexibility | Transparency | Figs |
| 90% w/w PVP and 10% w/w glycerol | Moderate | medium | 9A |
| 100% w/w HPC | Moderate | high | 9B |
| 90% w/w HPC and 10% w/w glycerol | High | high | 9C |
| 90% w/w CGN and 10% w/w glycerol | Moderate | low | 9D |

In the next experiment, film comprising 39% (w/w) PAA 50% (w/w) HPC, 10% (w/w) glycerol, and 1% (w/w) dyclonine were prepared. 445 mg HPC and 100 mg glycerol were mixes, 445 mg PAA and 10 mg dyclonine were added and mixed, and 100 mg of the powder mixture was compressed in 2.4 cm round mold under 10 tons force. The films were fixed to the bottom of 20 ml scintillation vials, using 50 µl of, 3 ml phosphate buffer 6.8 pH was added to the vials at 37° C., the release medium was changed every 10 min, the solutions were saved for content determination.

In the first 30 min, the film was intact and constantly releasing dyclonine. After this period, the film started to fall apart and dissolve. The results are shown in FIG. 9.

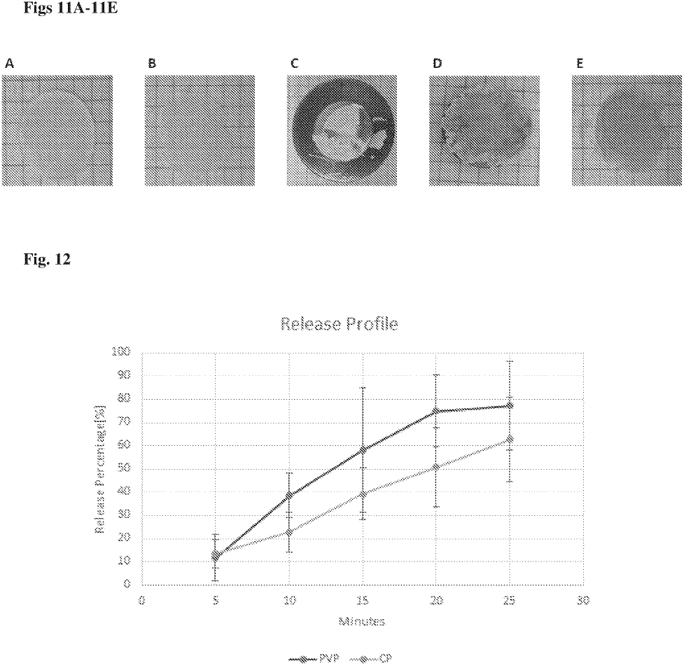

Films of various thickness and weight were prepared, combining 71.2% (w/w) CP, 8.9% (w/w) HPC, 8.9% (w/w) CGN, 1% (w/w) dyclonine and 10% (w/w) glycerol, by mixing glycerol,HPC and CGN and then adding the other of the components, and compressing the mixture in 2.4 cm round mold under 10 tons force. The three types of films are shown in FIG. 10. The 80 mg film had 180 µm thickness, was flexible, and had medium transparency. The 160 mg film had 360 µm thickness, was less flexible and had lower transparency. The 240 mg film had 530 µm thickness, was less flexible and less transparent. All films survived 20 bending tests. Films (100 mg) with various compositions of polymers and 10% glycerol are shown in Table 17 and FIGS. 11A-11E.

TABLE 17

Flexibility and transparency of the films with various polymers

| | | |
|---|---|---|
| Components (90%) | Transparency, flexibility, and thickness | Figs |
| Microcystalline cellulose | Medium transparency, low flexibility, 150 microns thickness, high glycerol absorbance | 12A |
| Carboxymethyl cellulose(CMC) | medium transparency, brittle | 12B |
| Eudragit s100 | High glycerol absorbance, brittle | 12C |
| Pullulan | Low transparency, highly flexible, high glycerol absorbance | 12D |
| Pectin | No transparency, no glycerol absorbance, crumbly. After adding 15% [w/w] HPC became low transparency and flexible, soft | 12E |

Additional types of 100 mg films with various compositions of ingredients are shown Table 18. Films were compressed under 3 tons in 2.4 cm round mold for 30 sec. All films were almost transparent and highly flexible.

TABLE 18

Types of 100 mg films with various components

| # | Complementary [45.5% w/w] | HPC [% w/w] | Glycerol [% w/w] | 2,4 dichlorobenzyl alcohol [% w/w] |
|---|---|---|---|---|
| PVP | Polyvinylpyrrolidone | 45.5 | 10 | 1 |
| CG N | k-carrageenan | 45.5 | 10 | 1 |
| CP | Poly(acrylic acid) | 45.5 | 10 | 1 |

The films were tested for the release of 2,4 dichlorobenzyl alcohol. The release test was performed as above using UV spectrometer at 271 nm. Measurements were performed in triplicates.

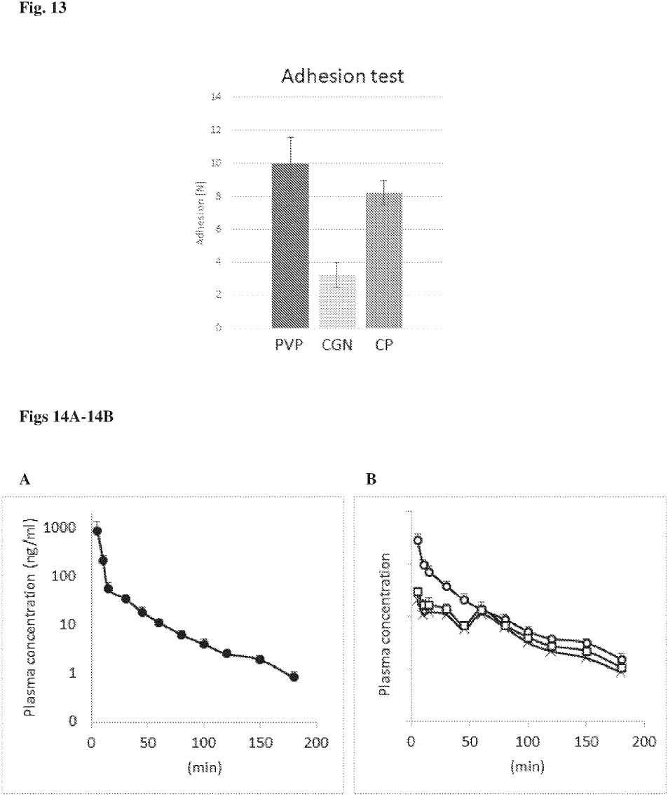

The CGN film dissolved in 5 min, the PVP film dissolved in 25 min and the CP film did not dissolved after 30 min but detached from the vial. The release profiles from PVP and CP films are shown in FIG. 12.

In the adhesion test, each film was attached to a glass slide using ethyl cyanoacrylate adhesive then fixed the slide to upper arm into slide house with bolts, also another flat glass slide fixed to the lower substrate using bolts and (2.6×3.8 cm). The flat glass slide was wetted by 50 µl DDW and the tablet was lowered at a rate of 1 mm/s until normal load of 5 N. The force was maintained for 10 sec to measure the maximum adhesion force, the machine pulled the upper arm at a constant rate of 5 mm/s and the force was recorded until detachment. Adhesive force was determined at the maximum point as an average of five independent measurements for each substrate and film pair. PVP and CP films showed good adhesion The results are shown in FIG. 13.

Conclusions: Flexible films can be obtained with at least 50% w/w HPC and 10% glycerol. The HPC increases the transparency of the film. PEG400 and propylene glycol are marginally suitable as plasticizers. The overall durability of the film and the drug release profile depend on the composition. PVP and CP have strong adhesion strength compared to CGN.

### Example 2 Buccal Delivery of Apomorphine (APO)

### 2.1 Materials and Methods

2.1.1 HPMC-based core ABPD preparation: All polymers were sieved through a 50-mesh sieve prior to their use. 70.0 mg APO, 2.3 mg ANT, 30.0 mg ascorbic acid and 50.0 mg HPMC were triturated using a mortar and pestle. 0.75 mg Mg stearate were added and lightly mixed. A 1.6 mm thick cylindrical tablet was prepared by direct compression using a laboratory IR press (Perkin Elmer) fitted with a 10 mm flat-faced punch and die set, applying a pressure of 2 ton/cm² for 30 sec. This tablet was placed in the middle of 13 mm flat-faced punch and die set. A homogenous mix of 83.0 mg hydroxypropyl cellulose (HPC) and 167.0 mg Carbopol 934 (CP) with 1.25 mg Mg stearate (lightly mixed) were poured over the tablet into the die. 2.6 mm thick core-shell cylindrical tablet was prepared by direct compression of 2 ton/cm² for 30 sec.

2.1.2 Witepsol H15-based core ABPD preparation: 69.0 mg APO, 2.0 mg ANT, 4.5 mg ascorbic acid, 69.0 mg witepsol H15 and 8.85 mg tricaprin were heated to 50° C. with constant stirring to obtain a liquid homogenous dispersion. The dispersion was slowly cooled with constant vigorous stirring to room temperature to obtain a solid. This solid was introduced into 8 mm flat-faced punch and die set. A cylindrical tablet was prepared by direct compression of 2 ton/cm² for 30 sec. This tablet was placed in the middle of 13 mm flat-faced punch and die set. A homogenous mix of 100.0 mg HPC and 200.0 mg CP with 1.5 mg Mg stearate (lightly mixed) were poured over the tablet into the die. A 2.9 mm thick core-shell cylindrical tablet was prepared by direct compression of 2 ton/cm² for 30 sec. The diameter of the core was changed to 10.5 mm due to compression.

2.1.3 APO release from hydrogel-based core testing: 40.0 mg APO, 1.0 mg ascorbic acid and 1.6 mg K₂HPO₄ were added to 880 µl 50° C. DDW. 100.0, 120.0 or 150 mg fish gelatin were added. No gelatin was added to the fourth group. All work was done in triplicates. After vigorous stirring full dissolution to homogenous solution was achieved. The pH of the solutions was adjusted to 5.9 by H₃PO₄ and KOH. 0.5 ml of each solution was introduced into dialysis bag (Specta/PorⓇDialysis membrane, MWCO 25000, flat width 12 mm). The bags were submerged into a beaker with 100 ml 0.1% ascorbic acid solution adjusted to pH 5.9 by NaOH and HCl. The solutions were held at 34° C. (±1) with constant stirring. 100 µl samples were taken at time points 0, 10, 25, 40, 60, 90, 120, 150 and 180 min into a vial containing 1 µl of 10%^{v}/ᵥ mercaptoethanol ethanolic solution. 100 µl of fresh receiving solution was added to the beaker to retain sink conditions. The samples withdrawn were centrifuged for 10 min at 18626 g and 50 µl of the supernatant were injected into a HPLC system.

2.1.4 Chromatographic analysis of in-vitro samples: The samples were injected into Waters 2695 Separation Module HPLC system coupled with Waters 2475 Multi Gamma Fluorescence Detector (Waters Co., MA, USA), into BDS Hypersil Cyano (Phenomenex, CA, USA) column (5 µm, 100 Å, 250 mm × 4.6 mm) protected by Hypersil Cyano (Phenomenex, CA, USA) pre-column (5 µm, 4 mm × 10 mm). Flow rate was set to 1.2 ml/min and temperature to 30° C. Mobile phase was comprised of 55:45 ACN : phosphate buffer (in 1 L DDW: 2.42 g NaH₂PO₄*H₂O, 0.28 ml H₃PO₄, 45.45 mg EDTA; pH adjusted to 3.0 using NaOH and H₃PO₄). Run time was 7 min. Detection was done at excitation wavelength of 270 nm and emission 450 nm. Retention time for APO was ~4.0 min. Data acquisition and analysis were performed with Empower Pro software (2002, Waters). Linearity curve (2.5-250 µg/ml, R²>0.997) was performed before the batch of samples and used for quantification.

2.1.5 Hydrogel-based core ABPD preparation: 1500 mg mannitol and several grains of methyl red where triturated using mortar and pestle to fine homogenous pink powder. A cylindrical tablet was prepared by direct compression in a 13 mm flat-faced punch and die set, applying a pressure of 2 ton/cm² for 30 sec. This tablet was placed in the middle of 20 mm flat-faced punch and die set. A homogenous mix of 566.7 mg HPC and 1133.3 mg CP with 8.5 mg Mg stearate (lightly mixed) were poured over the tablet into the die. 8 mm thick core-shell cylindrical tablet was prepared by direct compression of 3 ton/cm² for 30 sec. The mannitol core (easily differentiated by a pink color) was scraped out from the HPC:CP shell by a spatula. The inner side of the shell was covered by a parafilm® M. 38.3 mg APO, 1.0 mg ANT, 1.0 mg ascorbic acid, 1.6 mg K₂HPO₄ and 120.0 mg fish gelatin were added to 50° C. DDW and vigorously stirred until full dissolution to homogenous solution. The pH of the solution was adjusted to 5.9 by H₃PO₄ and KOH. The solution was cooled at room temperature for 10 min with continuous stirring. Approximately 750 mg of the solution was poured (over analytical scales) into previously described shell and stored refrigerated.

2.1.6 ABPD shell disintegration test: 6 cylindrical tablets containing homogenous mix of 566.7 mg HPC and 1133.3 mg CP with 8.5 mg Mg stearate (lightly mixed) were prepared by direct compression in a 20 mm flat-faced punch and die set, applying a pressure of 3 ton/cm² for 30 sec. The tablets were introduced into dissolution apparatus (Erweka® DT6R, Heusenstamm, Germany) each into a glass containing 300 mg K₂HPO₄, 584 mg NaCl, 55 mg CaCl₂*H₂O and 250 mg ascorbic acid in 250 ml DDW; pH adjusted to 6.2. Temperature was adjusted to 34° C. (±1) and a constant stirring was set to 50 rpm. Visual inspection of the disintegrating tablets was done every hour for 12 h.

2.1.7 In-vivo protocol of IV administration: Domestic female pigs (sus scrofa domesticus) of 35-40 kg were allowed to acclimate before the experiment. Pigs were fasted overnight before the experiments. On the morning of the experiment pigs were anesthetized by xylazine 1 mg/kg, midazolam 0.04 mg/kg, ketamine 5 mg/kg and propofol 4 mg/kg. Pigs were positioned on their back. Endotracheal tube was inserted, and pigs were respired with oxygen. The anesthesia was maintained by 1.9% isoflurane at 1 L/min rate. Catheter was inserted into ear vein through which physiological saline was administered. Pigs received amoxicillin 15 mg/kg antibiotic and tolfenamic acid 2 mg/kg analgesic. An intra-jugular catheter was inserted for blood sampling. Pigs were constantly monitored for their respiration rate, rectal temperature, O₂ saturation, heart rate and blood pressure. Cardiovascular parameters were obtained by blood pressure monitor with porcine adapted front leg cuff. A solution of 10 mg/ml APO and 0.2 mg/ml ascorbic acid (as an antioxidant) in DDW, freshly prepared on the morning of the experiment, were administered through the jugular catheter, by an approximately 1 min infusion, to 4 pigs with mean weight of 41 kg (SEM ±1.4). The administered dose of APO was 1 mg/kg. Additional 10 ml physiological saline was administered by a 1 min infusion through the jugular catheter to add the remaining APO dose in the catheter into the blood stream. Here and further all utensils, whenever possible, were made of polypropylene to prevent APO adsorption to glass and other types of plastic. 10 ml blood samples were taken through the jugular catheter into heparinized tubes containing 10 mg ascorbic acid 10 min before the administration of APO to pigs and after administration at 5, 10, 15, 30, 45, 60, 80, 100, 120, 150, 180 and 210 min. The blood was centrifuged immediately after the sampling at 2540 g for 10 min. Plasma was collected into tubes containing 10 µl mercaptoethanol and stored at -80° C. pending analysis. Isoflurane anesthesia was terminated, and pigs were recovered under veterinary surveillance to consciousness.

2.1.8 In-vivo protocol of buccal administration: Following full recovery of 1 week, these (and additional) pigs underwent experiments with administration of buccal devices. The preparation of pigs for the experiments were done as previously described. Pigs were positioned on their left side. Inner side of the left cheek was gently cleaned with a gauze. ABPDs were adhered to the inner side of the left cheek and pressed with finger for 1 min, to insure full adhesion. ABPDs were sprinkled by physiological saline every 15 min as a simulation to the saliva flow. 10 ml blood samples were taken and treated as previously described 10 min before the administration of APO to pigs and after administration at 0.25, 0.5, 0.75, 1, 1.33, 1.67, 2, 3, 4, 5, 6, 7, 8, 8.33, 8.67, 9, 9.5, 10, 10.5, 11, 11.5 and 12 h. At 8 h, ABPDs were removed and stored at -80° C. pending analysis for APO remaining in the devices. The liquefied core was carefully collected and stored at -80° C. After the ABPDs removal, buccal tissue was carefully cleaned with a gauze, so that no APO will remain on buccal surface. At 12 h, pigs were euthanized by injection of overdose of KC1. Cheeks were excised. Half of the excised cheeks were stored at -80° C. pending analysis for APO remaining in the tissue. The other half was fixated in 4% formaldehyde and stored for at least a month. Afterwards, these samples were trimmed, embedded in paraffin and sectioned. They were stained with Hematoxilyn and Eosin and underwent a histopathological examination. As a first step, each of the first 3 pigs received a different ABPD (as previously described): hydroxypropyl methylcellulose (HPMC) - based core, witepsol H15 - based core, or hydrogel - based core. After results evaluation, 3 additional pigs received hydrogel -based core ABPDs. Lastly, 1 pig received a hydrogel-based core ABPD with modified parameters.

2.1.9 APO and ANT extraction from plasma: 10 µl metoprolol methanolic solution (100 ng/ml) as an internal standard was added to 1.5 ml thawed plasma samples and vortex-mixed for 1 min. 350 µl Na₂HPO₄ (0.2 M) were added, followed by vortex-mixing for 2 min. 6 ml of diethyl ether was added followed by vortex-mixing for 5 min and centrifugation at 2540 g for 5 min. Supernatant was transferred to clean tubes and evaporated to dryness under vacuum. The samples were reconstituted in 100 µl 1:4 MeOH:DDW solution containing 0.01%^{v}/ᵥ formic acid and 0.1%^{v}/ᵥ mercaptoethanol and injected into LC-MS-MS system.

2.1.10 APO and ANT extraction from buccal tissue: After the submucosa was separated from the mucosa, tissue samples were cut into small pieces of several millimeters and placed into tubes. 3 ml of 0.1 M HCl, 10 µl metoprolol methanolic solution (100 ng/ml), 40 µl of ascorbic acid aqueous solution (100 mg/ml) and 20 µl of mercaptoethanol were added. The tubes were vigorously shaken for approximately 20 h. Afterwards, the tubes were centrifuged at 2540 g for 10 min. The supernatant was transferred to a clean tube, to which 0.3 ml of 1M NaOH and 0.7 ml 0.2 M Na₂HPO₄ were added. The tubes were vortex-mixed for 2 min. The pH was adjusted to 7 - 9 by NaOH and HCl. 6 ml of diethyl ether were added, tubes were vortex-mixed for 5 min and centrifuged at 2540 g for 10 min. The supernatant was transferred to a clean tube and evaporated to dryness under vacuum. 3 ml of 0.1 M HCl were added to the tissue once again and vigorously shaken for 20 min. Afterwards, the tissue underwent the same procedure one more time with supernatant collected to respective tubes as previously. After evaporation to dryness, the samples were reconstituted in 100 µl of 1:4 MeOH : DDW solution containing 0.01%^{v}/ᵥ formic acid and 0.1%^{v}/ᵥ mercaptoethanol. Samples were 2000-fold diluted in the reconstitution diluent and injected into LC-MS-MS system.

2.1.11 APO and ANT extraction from ABPD core: The hydrogel-based core solutions were 10,000,000-fold diluted in the reconstitution diluent and injected into LC-MS-MS system.

2.1.12 Chromatographic analysis of in-vivo samples: The chromatography was performed using a Shimadzu UHPLC System. The chromatographic separations were performed on a Kinetex™ (Phenomenex, CA, USA) column (EVO C18, 2.6 µm, 100 Å, 100 × 2.1 mm), protected by a SecurityGuard™ (Phenomenex, CA, USA) ULTRA cartridge (C18, 4 × 2 mm). The injection volume was 5 µl, temperature was 40° C. The chromatographic separation was achieved using linear gradient program at a flow of 0.3 ml/min over a total run time of 18 min. The results are shown in Table 19.

TABLE 19

Chromatographic analysis of in-vivo samples

| Time (min) | Solvent A (%) | Solvent B (%) |
|---|---|---|
| 0.0 | 95 | 5 |
| 9.0 | 65 | 35 |
| 10.0 | 2 | 98 |
| 14.0 | 2 | 98 |
| 14.5 | 95 | 5 |
| 18.5 | 95 | 5 |

Gradient program: Solvent A is 0.01%^{v}/ᵥ formic acid in DDW and solvent B is MeOH.

The first 1.3 min and last 8.0 min of the column effluent were diverted to waste. 1:1 mixture of MeOH:DDW was used for washing the needle prior to each injection cycle. All samples were analyzed in duplicates. The analytes were detected by a Sciex (MA, USA) Triple Quad™ 5500 mass spectrometer in positive ion mode using electrospray ionization (ESI) and multiple reaction monitoring mode of acquisition. Air was produced (SF 2 FF compressor, Atlas Copco, Belgium) and purified using an NM20Z nitrogen generator (Peak Scientific, Scotland). Purified air was used as source and exhaust gases and purified nitrogen as curtain and collision gases. A receiver was placed between the compressor and the nitrogen generator for a large and stable supply of air.

For transitions of analytes, the molecular ion of the compounds [M+H]⁺ was selected in the first mass analyzer and fragmented in the collision cell followed by detection of the products of fragmentation in the second mass analyzer. The TurboIonspray® probe temperature was set at 600° C. with the ion spray voltage at 5.5 KV. The curtain gas was set at 30.0 psi. The nebulizer and turbo heater gases were set to 40 psi and the collision gas was set to 8 psi. The entrance potential was set at 10 V. The dwell time was 30 msec. The data is summarized in Table 20.

TABLE 20

Transitions of analytes

| Name | Precursor (m/z) | Product (m/z) | DP (V) | CE (eV) | CXP (V) | Rt (min) |
|---|---|---|---|---|---|---|
| APO | 268.0 | 237.0 | 76 | 23 | 24 | 6.4 |
| | | 191.0 | 76 | 43 | 24 | |
| APO-sulfate* | 348.0 | 237.0 | 76 | 30 | 16 | 6.3 |
| | | 268.0 | 76 | 15 | 16 | |
| APO-glucuronide* | 444.0 | 237.0 | 76 | 30 | 16 | 5.1 |
| | | 268.0 | 76 | 15 | 16 | |
| APO-quinone** | 266.0 | 235.0 | 56 | 19 | 28 | 6.4 |
| | | 207.1 | 56 | 33 | 20 | |
| ANT | 189.0 | 56.1 | 106 | 53 | 10 | 7.7 |
| | | 104.0 | 106 | 31 | 14 | |
| | | 96.1 | 101 | 35 | 14 | |
| Metoprolol | 268.0 | 116.0 | 21 | 27 | 16 | 8.4 |
| | | 98.2 | 21 | 25 | 8 | |

Multiple reaction monitoring transitions and parameters for the analytes in positive ion mode. The first transition for each analyte was the quantifier ion, the second transition was the qualifier ion.
m/z: mass to charge ratio; DP: declustering potential; CE: collision energy; CXP: collision cell exit potential; V: volts; eV: electronvolts; Rt: retention time.
* Due to the lack of standard solutions of APO-sulfate and APO-glucuronide, their transitions are based on literature settings. Two unresolved peaks were observed and putatively identified as 10- and 11-APO sulfate. The calculated area is the sum of their peaks.
** Due to the lack of standard solution of APO-quinone, the transitions, parameters and relative concentrations are based on the quinone present in APO standard solutions.

Data acquisition and analysis were performed with Analyst 1.6.2 software (Sciex). Linearity curve (0.033 - 3.333 ng/ml for APO and 3.75 - 37.5 ng/ml for ANT, R²>0.997) was performed before every batch of samples and used for quantification.

2.1.13 Statistical and PK analysis: All values are expressed as mean ±standard deviation (SD) for in-vitro studies or standard error of the mean (SEM) for in-vivo studies, or as otherwise specified. The concentration vs time in-vivo data was analyzed by a non-compartmental PK analysis using Phoenix WinNonlin 8.1 (Certara, USA). Following this analysis PK parameters were obtained.

2.1.14 Equations used for calculations: The flux (Jss) of the test substance through the buccal mucosa is calculated by the Eq. 1:

$$\text{Jss} \text{=} \left(\text{dQ} / \text{dt}\right) / \text{A} \qquad \text{(1)}$$

where dQ/dt is the amount of test substance permeated over time and A is the area of buccal mucosa available for permeation. Plasma levels at steady state (Css) of the test substance following in-vivo buccal administration are calculated by Eq. 2:

$$\text{Css} \text{=} \text{Jss} / \text{CL} \qquad \text{(2)}$$

where CL is the PK parameter of total clearance of the tested substance in-vivo.

Apparent permeability (Papp) is calculated by Eq. 3:

$$\text{Papp} \text{=} \left(\text{dQ} / \text{dt}\right) / \left(\text{A*Co}\right) \qquad \text{(3)}$$

where Co is the initial concentration of the test substance near the buccal mucosa in the ABPD at the beginning of the experiment.

Bioavailability of a test substance (F) is calculated by Eq. 4:

$$\text{F} \text{=} \left(\text{CL*AUC}\right) / \text{D} \qquad \text{(4)}$$

where AUC is the area under the curve of plasma concentration vs time plot and D is the dose of the test substance administered.

### 2.2 Results

Prolonged release (PR) delivery of apomorphine (APO) through the buccal mucosa has high potential as a novel therapeutic method for treating PD by substituting the current parenteral infusions. Pig is a convenient animal model for buccal permeability research due to its morphological and permeation rate similarities to humans. The hypothesis of this work was that delivering APO in a buccal mucoadhesive PR device of the invention will enable to obtain therapeutically relevant plasma levels, thus proving a potential non-invasive substitute to the currently used parenteral infusions in PD patients.

Three types of APO buccal mucoadhesive prolonged release delivery devices (ABPDs) were tested in-vivo. The PK data on the obtained APO plasma levels are discussed in comparison with the PK data on the IV APO administration and the behavior of antipyrine (ANT) added to every ABPD. ANT is a known permeation marker of intestinal drug delivery. It was highly valuable in understanding the release kinetics and buccal permeation processes of APO. It has high passive transcellular diffusion through biological membranes. As opposed to APO, it is a hydrophilic molecule, unionized at physiologic pH, and has low plasma clearance (CL).

### 2.2.1 IV Administrations of APO to Pigs

FIG. 14A shows mean (±SEM) APO plasma concentration vs time following IV administration of 1.0 mg/kg dose of 10.0 mg/ml aqueous solution administered by 1.0 -1.5 min infusion to four female pigs. PK parameters were calculated and compared to hominine obtained from literature (Table 21). The data indicate that CL in pigs is approximately 3-fold higher than in human, due to shorter half-life and larger volume of distribution. Applying Eq. 2, it was deducted that in case the APO flux through porcine and hominine mucosa is similar (although theoretically it should be higher through thinner hominine mucosa), plasma levels of APO in human will be proportionally about 3-folds higher as well. Therefore, if the intension is to obtain APO plasma levels of ~2 ng/ml (which is the lower part of therapeutic window for PD treatment) in 70 kg human, it should be aimed at experimentally reaching blood levels of approximately 0.67 ng/ml in the plasma. By inserting into Eq. 2 Css value of 0.67 ng/ml and CL value of 13.1 L/h/kg (as currently obtained) for pig weighting 41 kg, the required Jss was calculated to be 0.36 mg/(cm²*h). As was previously shown, the Papp of APO through excised porcine buccal mucosa in Ussing diffusion chamber equals 3.00* 10⁻⁶ cm/sec at pH 7.4 and 0.51* 10⁻⁶ cm/sec at pH 5.9. For prolonged release of APO, it would be preferable to use pH 5.9. In that case, the Jss at maximum APO solubility in the hydrogel of 40 mg/ml will be 0.073 mg/(cm²*h) (Eq. 3). As this Jss is approx. 5-fold lower than the required to obtain APO plasma levels of 0.67 ng/ml, it is needed to increase the exposed buccal area for absorption from 1 cm² to 5 cm². That is feasible, as the available buccal mucosal area for absorption is ~50 cm².

FIG. 14B shows profiles of peak areas’ ratios of metabolite to internal standard vs time for three main (inactive) metabolites of APO: APO-quinone (autoxidation product), APO-glucuronide and APO-sulfate (the latter two are products of direct Phase II metabolism). Due to lack of standards, quantification could not be obtained for peak areas from the chromatogram, but only a plot of profile trends as a function of time. From these profiles, half-lives of metabolites were calculated as (mean ±SEM): 25.5 (±2.0) min for APO-quinone, 32.2 (±1.8) min for APO-glucuronide, and 29.2 (±1.3) min for APO-sulfate. By applying one-way ANOVA with Tukey post-hoc analysis, statistical difference with p<0.05 was found between half-lives of APO to APO-sulfate and APO-glucuronide.

TABLE 21

Pharmacokinetic data

| | CL (L/h/kg) | Vd (L/kg) | t_{½} (min) | AUC_{0-inf} (ng*h/ml) |
|---|---|---|---|---|
| Pigs | 13.1 (±2.4) | 6.6 (±1.2) | 21.3 (±1.3) | 107.4 (±43.4) |
| Human | 3-5 | 1-2 | ~40 (30 - 90) | |

Mean (±SEM) pharmacokinetic parameters obtained after administration of 1.0 mg/kg dose of 10 mg/ml apomorphine in 1.0-1.5 min intravenous infusion to pigs compared to reported in literature for human.

Table 22 shows safety profiles following the APO administration. Several vital parameters were monitored and compared to normal as described in literature. The table shows mean of the parameters with the lowest and highest values detected throughout all four pigs. All the parameters were at normal range, except diastolic pressure which was lower than normal. Nevertheless, it was always constant in all four pigs, from the beginning of the experiments to their end, when APO was fully eliminated.

TABLE 22

Safety profiles

| | Rectal temp (°C) | Heart rate (beats/min) | Systolic blood press (mmHg) | Diastolic blood press (mmHg) | O2 saturation (%) | Respiration rate kept at (min⁻¹) |
|---|---|---|---|---|---|---|
| Obtained | 38.2 (37.4 - 39.4) | 95.2 (74 - 120) | 81.7 (72 - 101) | 43.1 (32 - 55) | 97.7 (95 - 100) | 12.2 (11- 13) |
| Literature | 36.7 - 39.2 | 60 - 140 | 73 - 230 | 52 - 165 | ≥95 | 6-20 |

Monitored vital parameters in pigs following administration of 1 mg/kg dose of 10 mg/ml apomorphine in 1.0 - 1.5 min intravenous infusion. Presented as a mean with lowest and highest values detected throughout all four pigs and compared to reported in literature.

### 2.2.2 Dissolution Test of the Shell

Visible swelling of the shell was obtained after 30 min from the beginning of the experiment. After 4 h, the shell swelled by 2-fold of the original size and started to shear small parts. After 8 h, no additional increase in size was observed, but significant shearing of small particles was clearly visible. After 11 h, the shell was completely disintegrated.

### 2.2.3 APO Release From Gelatin Matrix

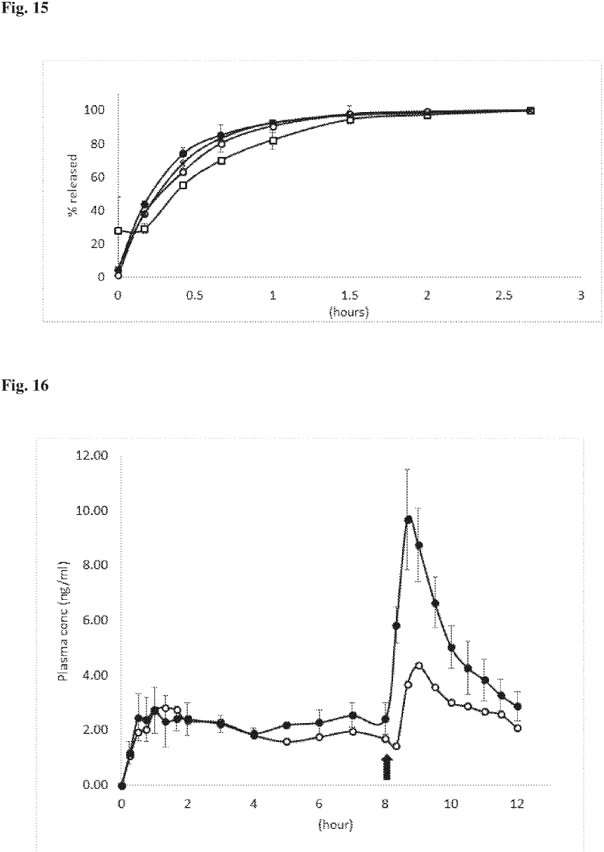

FIG. 15 shows release profiles of 20 mg APO from 0.5 ml hydrogel (pH 5.9) with various concentrations of gelatin through dialysis bag into aqueous medium. It shows that slight effect of the gelatin concentration on the APO release rate with no significant differences between the tested samples and the formulation without gelatin. At 1.5 h, all APO dose was released. For the preparation of ABPD, 12%^{w}/_{w} concentration of gelatin was chosen as the core matrix.

### 2.2.4 In-vivo Studies With 3 Types of ABPDs

The shell of all three types of ABPD were subjected to limited degree of swelling but remained in their form as at the beginning of the experiment. They were relatively strongly stuck to the mucosa, without visible danger of detaching, even after application of light manual force. Nevertheless, they were relatively easily removed manually at predetermined time points during 8 h. No APO was detected in the plasma after administering ABPDs with HPMC or witepsol H15-based cores. After ABPD removal from the buccal mucosa at 8 h, HPMC -based core remained intact and full dose of APO was recovered. Witepsol H15 core fully liquefied, however all APO dose remained on the mucosa in the form of white precipitate. FIG. 16 shows plot of APO concentration in plasma vs time following application of ABPD containing ~2.0 mg/kg dose of 38.30 mg/ml APO in hydrogel-based core, with exposed area for absorption of 4.74 cm² to four female pigs with average weight of 43 kg. The plateau with mean Css (±SD) of 2.36 (±0.2) ng/ml was reached after 30 min and remained relatively constant for 8 h. After removal of ABPD at 8 h, immediate elevation of APO plasma levels was observed expressed by Cmax of 9.7 ng/ml (at 8.7 h). APO plasma levels decreased shortly after with calculated half-life of 150.0 (±37.4) min which is ~7-fold slower than the half-life of APO obtained after IV bolus administration. AUC_{0-inf} was calculated as 50.7 (±7.9) ng*h/ml. Applying Eq. 4 and the data obtained after IV administration, F was ~20%. Therefore, the amount of APO reaching the blood circulation is ~17 mg. The content of hydrogel in the cores of removed ABPDs was tested to determine the APO amount. It was found that only 25-35% of the dose was released. In this case, the calculated F for APO available for permeation through buccal mucosa after release from ABPD was 55-80%. AUC₈₋₁₂ which is the approximate area of the peak obtained after ABPD removal was 32.8 (±6.7) ng*h/ml, which is about 65% of the total AUC. The APO extracted from buccal tissue exposed to ABPD was ~0.3 mg per pig.

### 2.2.5 Simulation of the Results in Pig to Human Setup

Assuming that Jss is similar through porcine and hominine mucosae (although hominine is thinner and thus is more permeable, suggesting that the results are underestimated), simulation of Css was performed for 70 kg human after ABPD application at the same experimental conditions and considering the CL difference from Eq. 2. The Css obtained was approximately 4-6 ng/ml.

### 2.2.6 Ex-Vivo - In-Vivo Correlations

Ex-vivo - in-vivo correlation to results obtained for APO permeation through excised porcine buccal mucosa in Ussing diffusion chamber was calculated. The Papp of APO obtained from ex-vivo study in Ussing system was 0.51*10⁻⁶ cm/sec at pH 5.9. Applying Eq. 3, at Co 38.3 mg/ml, Jss equals 0.07 mg/(cm²*h). Applying Eq. 2 for pig of 43 kg and buccal mucosal area available for permeation 4.74 cm², Css 0.59 ng/ml was obtained. This is 4-times lower than the Css obtained in current in-vivo experiment.

### 2.2.7 In-Vivo Study of Hydrogel-Based ABPD With Physical Parameters Modified

FIG. 16 further shows a plot of APO concentration in plasma vs time following application of ABPD with physical parameters modified to one female pig of 47 kg and 1.37 mg/kg dose of 18.8 mg/ml APO in hydrogel-based core with exposed area for absorption of 7.84 cm². Using the results obtained for the four pigs, theoretical Css 2.24 ng/ml is predicted (Eq. 2). The experimentally obtained Css for ABPD (±SD) is 2.15 (±0.44) ng/ml, which is very close to the predicted.

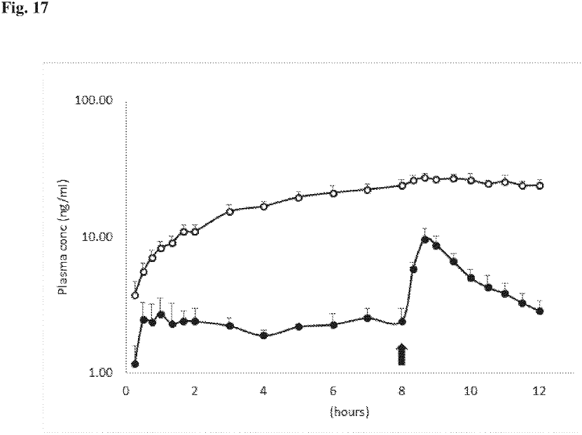

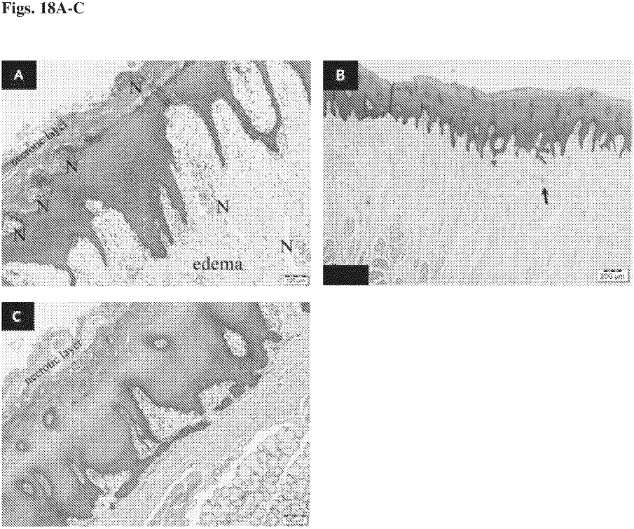

The plot for modified ABPD is very similar to the average of the four pigs, although the peak obtained after ABPD removal at 8 h is significantly reduced with 2-fold smaller Cmax. FIG. 17 shows semi-logarithmic plot of concentration vs time for the APO profile in FIG. 17 and ANT, the permeation marker added to the hydrogel-based core of ABPD (~0.05 mg/kg dose of 0.99 mg/ml ANT). The profile of ANT concentration vs time is different from APO. Despite lower dose and lower Co applied, ANT reached significantly higher plasma levels than APO. Plateau was not reached up to 8 h when ABPD was removed, although it was close. Following the removal of ABPD, only slight elevation in plasma levels of ANT was observed, as opposed to APO. Afterwards, during the next 3 h up to the end of the experiment, there was only slight decline in plasma levels. The extracted ANT from buccal tissue exposed to ABPD was ~0.07 µg per pig. Safety profile was similar to the obtained after IV APO administration (Table 22). No deviation in vital parameters was observed during the experiment. The diastolic blood pressure was in the same low range and constant through the experiment and not affected by the peak after the ABPD removal.

### 2.2.8 Histopathological Evaluation

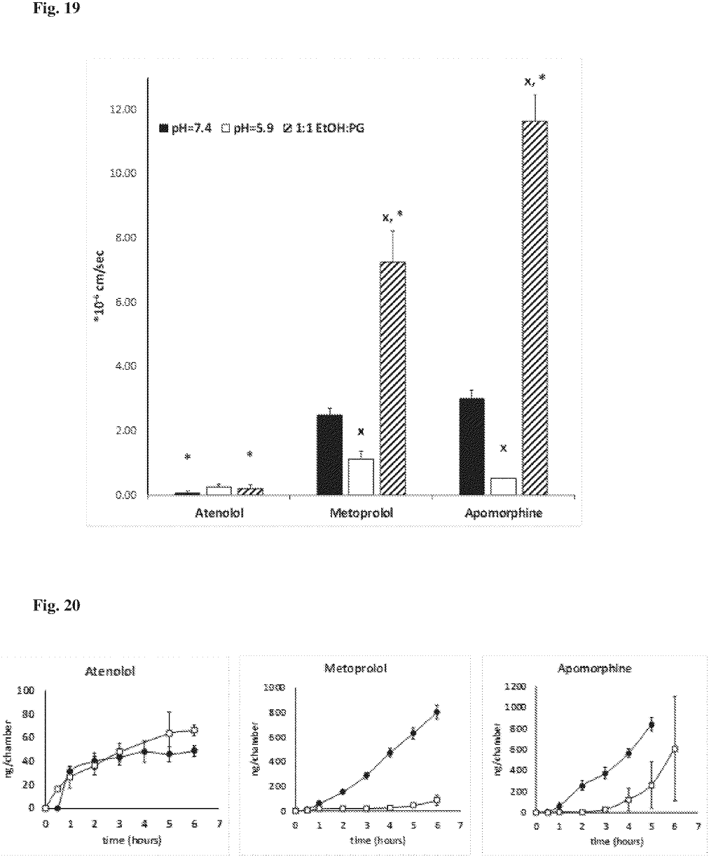

FIGS. 19A-19C show representative histopathological slides of buccal mucosal tissues exposed to ABPD vs non-exposed. The main observation is that the ABPD exposed mucosa shows minimal perivascular edema and aggregation of neutrophils, lymphocytes and rare eosinophils. It is intact and essentially normal. In the non-treated tissue and tissue exposed to HPC:CP-based ABPD shell, the most superficial layer of the epithelium consists of attenuated cells with pyknosis and necrosis.

### 2.3 Conclusions

It was hypothesized, that buccal mucosal PR delivery of APO has high potential as a novel non-invasive substitute for SC infusion with additional advantageous features that transcend over the infusion. To that end, the developed APO buccal mucoadhesive PR delivery devices were investigated in-vivo investigation in pigs as a model animal.

As the first step, to obtain PK profile of APO in pigs (due to lack of prior data), APO was administered to four pigs by IV bolus. It should be noted that that the CL of APO in pigs is 3-fold higher than in humans due to larger Vd and shorter half-life. While this presented a challenge to the development of a delivery device (as it is needed to administer significantly higher APO doses to be detectable in plasma), at the same time it assured than any relevant results obtained in the pig model will be of therapeutic relevancy for humans. By simulating with the PK data and APO Papp, the delivery device physical parameters were determined to acquire APO plasma levels at the lower part of therapeutic window. It should be noted that although maximal solubility of APO in deionized water is 20 mg/ml, a solubilization of up to 40 mg/ml in buffered hydrogel was achieved. This phenomenon is probably due to gelatin which produces a polymeric net enabling higher APO loading in the gel by suspending it to clear and homogenous semi-solid preparation. Ionic interaction between the positively charged amine group in APO molecule to negatively charged acid groups in the gelatin molecular structure cannot be ruled out. The hydrogel pH was selected to 5.9. As was previously shown, APO Papp at this pH is lower, and thus more prolonged release from the delivery device is anticipated. Nevertheless, by elevating pH, higher Css levels will be obtained, with somewhat degree of uncertainty about the length of delivery. This matter requires further investigation.

Three main APO metabolites (inactive) were monitored for their mass by LC-MS-MS. Although, quantification of the results could not be achieved due to lack of standards, a profile of plasma concentration vs time was obtained. Half-life of each metabolite was extracted. APO-quinone, the main metabolite produced by autoxidation, had a half-life equal to APO, meaning that its elimination is faster than its formation from APO with no accumulation expected. The other two metabolites (products of Phase II hepatic metabolism), APO-glucuronide and APO-sulfate, had half-lives longer than APO of 30 min, but still relatively short. Thus, accumulation to high and perhaps toxic levels should not be expected.

This issue was not problematic in the clinical setup with APO administered by SC infusion, knowing that APO metabolism in humans is almost full and only up to 4% excreted unchanged to urine. However, while metabolism after SC administration occurs in the liver after APO was distributed to other organs, with buccal administration there is a possibility of metabolism in the buccal mucosa before APO have reached the blood circulation. Therefore, there is a chance that slightly higher plasma levels of Phase II metabolism products will be present after this mode of administration. As it found that 55-80% of the dose released from ABPD reached blood circulation, metabolism in the buccal tissue cannot be ruled out. As was previously shown, after per oral administration, liver was the main metabolic organ of the first pass metabolism, unlike the gut. Yet only pharmacodynamic effects were observed rather than PK profile obtained. In that case, APO metabolism in the gut still cannot be rejected. There are metabolic processes in the buccal mucosa, and therefore, there is a place to suspect some degree of APO metabolism during the passage through buccal mucosa. Bearing in mind that possibility, addition of Phase II metabolism inhibitor to ABPD may enable elevation of APO bioavailability during buccal mucosal administration.

There were no significant safety issues in vital parameters monitored after IV administration of this rather APO high dose, as well as when ABPD was administered. The only one deviated from the normal range was diastolic blood pressure which was lower than normal. A known side effect of APO treatment is dose-related decrease in systolic and diastolic blood pressures. However, only less than 1% of patients had APO treatment withdrawn following severe orthostatic hypotension, hypotension and/or syncope in clinical trials. In addition, the fact that the blood pressure was low but remained constant in all pigs during the entire experiment after APO was fully eliminated may indicate measuring artifact. As the same results were obtained after administration of APO in ABPD with far lower blood concentrations for 12 h, it can be further concluded that low diastolic pressure is a measuring artifact rather than a safety issue, and that in current setup APO was safe.

In the next phase, three pigs received three different types of ABPDs. Each ABPD was constructed of the same outer HPC:CP-based shell and different core. Shell acts as a barrier to prevent APO escape into the oral cavity and as an adherer of the ABPD to the buccal mucosa. The three cores were: HPMC-based witepsol H15-based and hydrogel-based. ABPDs were adhered to buccal mucosa of anesthetized pigs and removed after 8 h, during that time shells underwent only limited degree of swelling. Although this contradicts the data obtained in dissolution tests, it can be explainable by the fact that in the dissolution test the shell was completely immersed in aqueous medium, while the exposure to water in the in-vivo study was very limited. During anesthesia there is almost no saliva production and the simulation by sprinkling saline every 15 min is apparently not enough.

Although this issue needs to be approached in the future development of the product, it is not likely that it had significant impact on the APO release from the core and subsequent permeation through buccal mucosa. In the pig receiving ABPD with HPMC-based core, no APO was detected in the plasma. After ABPD removal at 8 h, the core was intact without visible swelling or change in shape. All APO dose was recovered from the core. This suggests that there is not enough liquid to perform solubilize and release APO from the HPMC matrix. This is true especially due to obstruction of aqueous medium to arrive from the oral cavity by the shell, as well as by the fact that the mucosa of anesthetized animal becomes relatively dry. Nevertheless, it is logical to believe that the same results will be obtained in a conscious animal. This suggests that PR device protected from the saliva flow and intended to release active ingredient for permeation through the mucosa into the blood stream should be in a liquid state or should liquefy after being applied or should cause water influx from the mucosa (by osmotic pressure or some other method). Apparently, there are not enough liquids coming from the mucosa needed to solubilize the administered drug and/or the matrix (to enable drug release) as a first step required in the absorption process. APO was not detected also in the pig receiving ABPD with witepsol H15-based core. After ABPD removal at 8 h, the core fully liquefied due to body temperature as was expected, however all the APO dose that was dispersed in the witepsol H15 was located on the mucosa as a precipitate. This suggests that liquefaction alone of the matrix will not be sufficient, but rather the liquefaction of the matrix or other appropriate solute producing method can enable solubilization of the drug to molecular level and make it presentable to the mucosa to able permeation thereof.

APO plasma levels were further investigated in the hydrogel-based core ABPD. The hydrogel-based core is solid at room temperature and quickly liquefies at body temperature into clear aqueous solution with fully dissolved APO. After ABPD removal at 8 h, it was found that hydrogel was in a liquid form with no traces of visible APO precipitate. The obtained APO plasma levels indicated that the system performed as expected. Css was reached after 30 min, which fits the intended design of the PR device. The obtained Css of 2.36 ng/ml was surprisingly higher and more beneficial than expected from ex-vivo studies, as it suggested that a smaller device size can lead to the required plasma levels. By extrapolating to human, APO plasma levels of 4-6 ng/ml can be obtained at the same conditions - a value in the middle of the therapeutic window, thus suggesting that even a smaller device can be efficient for humans. Further, since human mucosa is thinner and therefore more permeable and due to the fact that at conscious state plasma levels are higher (due to improved blood flow and other factors), even higher APO plasma levels can be anticipated. Css was constant during 8 h and probably beyond that threshold, if not removed. Overall, the results were of therapeutic relevancy as a potential non-invasive treatment for PD patients to substitute parenteral infusions.

The significant peak in APO plasma levels was an unexpected finding, the peak appeared about 45 min after ABPD removal with 4-fold higher Cmax than Css, followed by a slow decline with the half-life of about 7-fold slower than after IV administration. The causes for this peak are not fully understood. It could be that it was produced by the minute stress associated with the detachment of ABPD from the buccal mucosa. It is rational to think that the peak appeared due to accumulation of APO in the buccal tissue. The AUC of the peak generates ~65% of total AUC. This significant contribution further ensures that its origin is from accumulation in the tissue. During ABPD removal, the mucosa and the whole pig’s cheek were moved and pressed causing APO to be pushed out of its storage place. The maneuvers with the cheek may have also caused an elevation of blood flow in the area, enabling the released APO to faster reach blood circulation. As no damage was caused to mucosa with all stratified layers intact, no breaching of the epithelium was caused. Further, the significantly slower half-life obtained also suits the conjecture that a ‘depot’ was produced in the tissue, slowly releasing APO into the blood, even after removal of the delivery device. The submucosal edema seen in the cheek exposed to ABPD may be a local adverse effect of APO or caused due to fact that pig lay on this cheek for 12 h during the experiment (as compared to non-exposed to ABPD cheek, which was facing upwards). This edema may also be the possible cause for the accumulating effect. As hominine buccal mucosa is thinner and especially in a conscious patient, may preclude the submucosal edema (as local blood flow is believed to be higher, so that the peak will be more modest, albeit not fully missing).

Unexpectedly, this finding offers new opportunities to further exploit the developed device. First, the slowed down elimination half-life enables prolongation of the APO exposure time, even when the device is needed or decided to be removed. Thus, the time needed to adhere the device the buccal mucosa may be substantially shortened. Second, the peak phenomenon may be advantageously utilized to cope with morning motor stiffness characteristic of PD patients. While ABPD worn overnight is removed (or self-disintegrated), and a newly adhered ABPD would be in its lag-time reaching Css, a boost of APO would be given by this peak effect to overcome the morning motor stiffness and bridge APO blood levels to the plateau. By applying Eq. 4 absolute bioavailability was calculated as ~20% which is far higher than the per oral which is less than 2%.

The content of ABPD core was tested after removal at 8 h to determine the amount of APO remained. It was found that only 25 - 35% of the initial dose were released from ABPD. As such, the APO bioavailability that left the device and reached the mucosa is as high as 55 - 80%. As previously discussed, this partial bioavailability may be due to possible metabolic processes inside the buccal mucosa. Yet, other unknown factors may also be the cause. The fact that 75 - 65% of initial APO dose did not leave ABPD should be addressed. Plasma Css is dependent on Jss of the tested substance (Eq. 1). As Css was stable during 8 h, it can be concluded that Jss remained also stable. Therefore, it can be deducted that as APO was depleted in the proximal to mucosa layers inside the core, it was replenished from more distal layers driven by concentration gradient. It is reasonable to assume that the remainder of APO dose in the ABPD core at 8 h was stored in the distal layers. Therefore, as this APO quantity was not required, it could have not been added from the beginning.

Regarding the setup, the width of the core (mucosal proximal to distal) could have been at least 2-times thinner. Decreasing the size of the device is desirable as smaller device is more convenient for use. Adjustment of the core width can affect the duration of APO release. Bearing in mind the possible ‘depot’ effect due to accumulation inside the buccal mucosal tissue and subsequent prolongation of exposure to APO, a further decrease in size is desirable. These issues should be addressed in further clinical studies. An in-vivo experiment done on one pig with the ABPD in which physical parameters were modified showed the ability to control APO blood levels by adjusting ABPD parameters. While APO concentration in ABPD was lowered, the exposed area for permeation was enlarged proportionally so that Css would remain unchanged. The result, as expected, showed that the device can be adjusted as needed to meet the therapeutic requirements with Css APO levels calculated and predetermined with high accuracy.

To establish ex-vivo - in-vivo correlation, the current Css value observed from the permeability point of view was compared to the previous ex-vivo studies. It was shown that ex-vivo study predicted 4-times lower result than the value obtained in-vivo. This discrepancy may be attributed to the fact that it is technically very difficult to fully isolate the mucosa from the underlying tissues ex-vivo. Some portion of lamina propria and even sub-mucosa will remain on the tissue mounted into Ussing chamber. Thus, additional membranes acting as barriers can slow down the permeability of tested agents, making Papp somewhat underestimated, as in the current case by the factor of 4. The barrier properties of layers underlying the mucosa were noted by others. Additional factors, such as lack of blood flow or adhesion to the chamber walls in Ussing system may contribute to this discrepancy.

Nevertheless, the results of Ussing chamber experiments are valuable, since showing the ability of a substance to permeate buccal mucosa in this system may suggests with a high degree of confidence a successful permeation in-vivo. To develop a scientifically proven model, additional agents with different physico-chemical properties should be studied for their ex-vivo - in-vivo correlation of permeability through buccal mucosa as a step towards ex-vivo screening of drug candidates for systemic administration through buccal mucosa. In addition, the use of permeation markers (e.g., metoprolol and atenolol) during Ussing experiments was proven to be of high importance, as while permeation result for a tested molecule can be distorted in ex-vivo study, it can be adjusted by comparing to a parallel result on a permeation marker. To understand the release kinetics from ABPD in-vivo, ANT permeation marker was added to the core. As opposed to APO, ANT is a hydrophilic molecule, but like APO, with a similar high permeability through biological tissues, specifically through the buccal mucosa. In addition, unlike APO, it has rather low CL enabling the acquisition of high plasma levels, as was observed in the present experiments. It explains the 10-fold higher plasma level reached at 8 h compared to APO Css, while ANT Co was 40 times lower. Only slight elevation in ANT plasma levels were obtained after ABPD removal at 8 h. As ANT is a hydrophilic molecule, no high tissue accumulation is to be expected. Thus, no peak was obtained after ABPD removal as opposed to APO, serving another proof that APO significantly accumulates in the buccal tissue. After the termination of the experiment at 12 h, buccal tissue exposed to ABPD was extracted to quantify the remaining amount of APO and ANT. The amount of APO in the tissue was ~ 100 times higher than ANT (with adjustment to dose applied), which is another proof to high APO tissue accumulation. Thus, incorporation of a permeation marker into ABPD have proven to be valuable, as it shed additional light on the kinetics of APO delivery via the buccal PR device.

Adding permeability markers in in-vitro experiments (e.g., when studying permeation through cell lines) and in ex-vivo experiments (such as Ussing experiments with intestinal segments) are standard procedures. However, this approach is not common in ex-vivo buccal studies and even less in-vivo, partially due to lack of general acceptance of specific molecules to act as buccal permeation markers. As was demonstrated here and previously utilizing permeation markers in the experiments adds validity to the obtained results and enables their better understanding. It seems logical that this approach should be further researched and developed, standardized, and implemented in future research.

This work clearly shows that the buccal mucosa presents a significant and important barrier for drug delivery as an administration route. Furthermore, it represents a rate limiting step in absorption of drugs which determines subsequent pharmacokinetic profile. When coming to develop a delivery device for buccal mucosal administration, one should take these factors into consideration. Knowing the physico-chemical properties of the investigated drug or agent, as well as its pharmacokinetics and considering the influence on the mucosal tissue is highly important when considering and developing the delivery device for systemic administration through buccal mucosa. Histopathology of the exposed tissues showed that ABPD did not cause any significant damage to the tissue. As has been noted, the observed edema can be explained by lack of movement and subsequent infiltration of immune cells.

### EXAMPLE 3 Prolonged Noninvasive Delivery of APO via Buccal Mucosa As Parenteral Infusion Substitute for Treating PD: a Preclinical Mechanistic Study

The present research aimed to determine the APO permeability rate through buccal mucosa using the PR delivery device of the invention. Permeability studies were done ex-vivo in Ussing diffusion chamber through freshly excised porcine buccal mucosa. Porcine buccal mucosa is highly similar to human by its morphology and permeability.

Several methods of permeability attenuation were studied to determine the permeability of APO at physiological conditions pH 7.4 and at pH 5.9 which is the lower limit of physiological pH in the mouth (compared to average of 6.8-7.4). Several works have shown the effect of pH on permeability of the buccal mucosa. Further, APO permeability was tested upon the addition of ethanol:propylene 1:1 glycol solution (1:1 EtOH:PG) to the mucosal side. Organic solvents were shown to enhance permeability of different molecules through the buccal mucosa. Moreover, this composition serves as the vehicle in Sativex® buccal spray, a registered pharmaceutical product for systemic delivery of delta-9-tetrahydrocannabinol and cannabidiol to buccal mucosa.

Finally, APO permeability was tested upon the addition of nano-liposphere formulation (NLF) - a blend of fatty acids and surfactants developed by the inventors. When administered into aqueous medium it instantly and spontaneously forms an oil in water nano-particulate emulsion. It was shown to significantly augment water dispersion of lipophilic molecules as well as their per-oral bioavailability through different pathways, including unstirred water layer passage, metabolism inhibition and shifting towards lymphatic pathway. Additionally, several constituents of this blend were shown to alternate buccal permeability of different substances.

Buccal permeability experiments of APO were undertaken with a novel ‘cocktail’ approach of simultaneous presence of two permeability markers, atenolol (ATN) and metoprolol (MTP). Both traverse the intestinal mucosa by passive diffusion. MTP, a moderately lipophilic molecule, passes by transcellular route and its permeability is rather fast. On the other hand, ATN is a hydrophilic molecule that passes by the paracellular route, and its passage is about 100 times slower. The FDA approves these beta-blockers as intestinal markers for permeability. As such, MTP is used as a reference for transcellular permeability, while ATN is a marker for paracellular permeability. There are no definite standard permeability markers for permeability research through buccal mucosa. However, permeability rates of MTP and ATN were previously studied and reported by different groups.

The second part of this work uses rats as in-vivo model for APO buccal delivery in-vivo. While rat is considered not a suitable model for buccal permeation research, since its mucosa is highly keratinized, few studies in rodents can be found. The logic behind working with rat, apart from the fact that rat is a more convenient model, is that a finding of permeability through rat mucosa, suggest with a high degree of confidence that the same result can be reproduced in the hominine mucosa. In addition, APO is considered a class 1 drug (Biopharmaceutics Drug Disposition Classification System) with a high rate of passive permeability through biological membranes, which can suggest a comparable permeability rate through keratinized mucosa.

### 3.1 Materials and Methods

3.1.1 Tissue preparation for permeability studies: Ussing diffusion chamber (VCC MC6 EasyMount, Physiologic Instruments Inc., USA) system was used for assessment of APO, MTP and ATN permeation rates through excised porcine buccal mucosa. Each chamber includes a heating block for temperature control and needle valves for adjustment of carbogen gas flow for oxygenation and gas lift stirring. Buccal tissues of female pigs, ~100 kg, were used in the experiments. Pigs were exposed to anesthetic agents, antibiotics, contrast agents and sacrificed by over exposure to KC1. To our knowledge there is no interference of these agents with our experimental protocol. Immediately following euthanasia, buccal tissue was removed and placed in ice-cold modified Ringer’s buffer (pH 7.4). Buccal mucosa segments were trimmed to 800-1000 µm by surgical scissors and tweezers and mounted into Ussing diffusion chambers. The exposed tissue surface area was 0.5 cm² and buffered solutions volume in each cell was 3 ml. The system was preheated to 35° C. Modified Ringer buffer was added to the receiver compartment and simulated saliva buffer to the donor side. The tissue oxygenation and the solution mixing were performed by bubbling with carbogen gas. The system was equilibrated for 30 min followed by replacing the solutions and adding 20 µl of the test solutions to the donor side of the chamber to commence the experiment.

3.1.2 Preparations of test solution and buffers: The simulated saliva buffer was prepared by mixing 105 mg NaHCO₃, 65.8 mg KH₂PO₄, 133.5 mg K₂HPO₄, 36.8 mg CaCl₂*2H₂O, 55.9 mg KCl and 250 mg ascorbic acid (as antioxidant of APO) in 250 ml DDW. pH was adjusted to 7.4 with NaOH and HCL. Modified Ringer’s buffer solution was prepared by mixing 6.54 g NaCl, 0.18 g CaCl₂*2H₂O, 0.37 g KCL, 0.24 g MgCl₂*6H₂O, 2.1 g NaHCO₃, 0.23 g Na₂HPO₄, 0.05 g NaH₂PO₄*H₂0, 1.44 g D-glucose, 0.36 g mannitol and 1 g ascorbic acid in 1000 ml DDW. pH was adjusted to 7.4 with NaOH and HCl. Test solution contained 16.2 mg ATN, 20.6 mg MTP and 19.2 mg APO which were dissolved in 4 ml methanol solution followed by vortex-mixing for two minutes. Solutions were freshly prepared on the days of the experiments. The simulated saliva buffer at pH 5.9 was prepared by mixing 105 mg NaHCO₃, 162 mg KH₂PO₄, 10.36 mg K₂HPO₄, 36.8 mg CaCl₂*2H₂O, 55.9 mg KCL and 250 mg ascorbic acid (as antioxidant of APO) in 250 ml DDW. pH was adjusted to 5.9 with NaOH and HCL. When testing the effect of organic solvents, 100 µl of the simulated saliva in the donor chamber were replaced by 100 µl of 1:1EtOH:PG solution. When testing the effect of NLF 70 µl of the simulated saliva in the donor chamber were replaced by 70 µl of NLF. NLF constitutes: 14.1%^{w}/_{w} Tween 20, 14.1%^{w}/_{w} Span 80, 14.1%^{w}/_{w} hydrogenated castor oil 40, 14.1%^{w}/_{w} tricaprin, 8.3%^{w}/_{w} egg lecithin and 35.4%^{w}/_{w} ethyl lactate. The constituents are melted at 37° C. and mixed until a clear yellow homogenous solution is obtained.

3.1.3 Experimental protocol: 100 µl samples were taken from receiver compartment at the beginning of the experiments, at half an hour and every hour until the end of the experiments. 100 µl of fresh Ringer’s buffer solution were added each time to receiver compartment. The dilution of the concentration of tested substances in the receiver compartment was accounted for. Samples were transferred into vials containing 0.2 µl mercaptoethanol ethanolic solution (100 µl/ml) as an antioxidant for APO. All samples were stored at -20° C. pending analysis. All the work was undertaken with polypropylene utensils to minimize the loss of APO due to its adsorption to glass and other plastics.

3.1.4 Chromatographic analysis: Samples were thawed, vortex-mixed for 2 minutes and centrifuged for 5 minutes at 18626 g. 10 µl of the supernatant were injected into the LC-MS system comprised of Waters 2695 Separation Module HPLC system, and Waters Micro-mass ZQ mass spectrometer (Waters Co., Milford, MA). The analysis conditions were as follows: XTerra MS (Waters Inc.) C18 Column (3.5 µm, 2.1 mm × 100 mm), temperature 45° C., flow rate 0.2 ml/min and mobile phase with a linear gradient as detailed in Table 23.

TABLE 23

Chromatographic analysis

| Time (min) | Acetonitrile+ 0.1% formic acid (%) | 0.1% formic acid |
|---|---|---|
| 0 | 5 | 95 |
| 10 | 80 | 20 |
| 13 | 5 | 95 |
| 30 | 5 | 95 |

Mobile phase gradient for LC-MS analysis of Ussing diffusion chamber permeability studies.

Retention times were as follows: ATN 4.2 min, APO 8.0 min, MTP 8.9 min. Detection was done at [M+H]+ with m/z: ATN 267.3, APO 268.3, MTP 268.3; at desolvation temperature of 350° C. and source temperature of 110° C., ion spray voltage of 3 KV, cone voltage of 22 V and extractor voltage of 3 V. Linearity was found for the three molecules between 1 - 1000 ng/ml with R²>0.997.

3.1.5 Statistical analysis of ex-vivo study results: Statistical analysis was performed by two-way factorial ANOVA with Tuckey post-hoc analysis simultaneously over all the results. Statistical significance was stated for p<0.05. All values are expressed as mean ±standard deviation (SD) or as otherwise specified. Papp calculation and simulations of steady state plasma levels for in-vivo testing

Apparent permeability (Papp) is calculated by Eq. 5:

$$\text{Papp} \text{=} \left(\text{dQ} / \text{dt}\right) / \left(\text{A*Co}\right) \qquad \text{(5)}$$

where dQ/dt is the amount of test substance permeated over time, A is the permeation area of buccal mucosa inside the Ussing diffusion chamber of 0.5 cm², and Co is the initial concentration of the test substance in the donor compartment at the beginning of the experiment.

The flux (Jss) of the test substance via the buccal mucosa is calculated by Eq. 6:

$$\text{Jss} \text{=} \left(\text{dQ} / \text{dt}\right) / \text{A} \qquad \text{(6)}$$

Simulation of blood levels at steady state (Css) of the substances if administered in-vivo is calculated by Eq. 7:

$$\text{Css} \text{=} \text{Jss} / \text{CL} \qquad \text{(7)}$$

where CL is the pharmacokinetic parameter of total clearance of the tested substance in-vivo.

3.1.6 In-vivo studies with rats under IV administration: 3 male Wistar rats ~300 g were kept under a 12-hour light/dark cycle with free access to food (standard rat chow) and water. Prior to surgery rats were fasted overnight with free access to water. Animals were anesthetized for the period of surgery by intra-peritoneal injection of 1 ml/kg of ketamine-xylazine solution (9:1, respectively). An indwelling cannula was placed in the right jugular vein of each animal for systemic blood sampling. After the surgery, rats were returned to cages overnight with free access to food and water. On the next morning, 2 mg/kg of APO (2 mg/ml in DDW) was administered as an intravenous slow bolus (over 30 sec) via right jugular cannula and washed with additional 0.5 ml of saline. Blood samples (0.3 ml) were withdrawn via the cannula 5 min prior to APO administration and at 2, 6, 10, 15, 20, 30, 45, 60, 75 and 90 min after APO administration and collected into microtubes with 2 µl heparin and 3.5 µl of ethanolic mercaptoethanol solution (10 µl/ml). The blood was centrifuged immediately after the sampling at 2540 g for 10 min, and plasma was stored at -80° C. pending analysis.

3.1.7 In-vivo studies with rats under buccal administration: Another 3 rats weighing ~330 g underwent the same chirurgical procedure, however without being awakened from anesthesia. After the completion of the surgical procedure rats were connected through the intravenous cannula to an infusion apparatus. An intravenous anesthetic solution was prepared by mixing 3.75 g dextrose, 675 mg NaCl, 10 ml ketamine (100 mg/ml) and 1.6 ml xylazine (20 mg/ml) in 150 ml DDW. The infusion rate was 30 (±5) µl/min with monitoring for absence of the paw-withdrawal reflex and by observing changes in the respiratory rate (optimal 60 - 90 breaths per minute) every 15 minutes as previously described. A hollow plastic tubes with inner area of 0.2 cm² were adhered by 2-octyl cyanoacrylate biological glue to rats’ cheeks. Into the tubes 400 µl of solution containing APO (7.6 mg), ascorbic acid (0.1 %^{w}/ᵥ) and K₂HPO₄ (0.2%^{w}/ᵥ) were injected. The solution was freshly prepared on the day of the experiment, pH adjusted to 6.2, and the solution filtered through 0.22 µm PTFE filters. Blood samples (0.3 ml) were taken via the jugular intravenous cannula, by temporally disconnecting it from the infusion apparatus. They were taken at 5 minutes prior to administration of APO and at 0.25, 0.5, 0.75, 1, 1.5, 2, 3, 4, 5, 6, 8 h after the administration. Blood samples were transferred into microtubes and treated as previously described. The drug solution administered to inner cheek was mixed every hour inside the tube for homogeneity assurance. 5 µl of the solution was taken at the beginning and at the end of the experiment.

3.1.8 APO extraction method from plasma: 10 µl mercaptoethanol ethanolic solution (100 µl/ml) and 10 µl benzo(a)pyrene ethanolic solution (1000 ng/ml) as an internal standard were added to 150 µl thawed plasma samples and vortex-mixed for 1 minute. 30 µl Na₂HPO₄ (0.2M) were added, followed by vortex-mixing for 2 min. 0.8 ml of diethyl ether was added followed by vortex-mixing for 2 minutes and centrifugation at 18626 g for 5 min. Supernatant was transferred to clean microtubes and evaporated to dryness. 0.8 ml of diethyl ether was added to the first set of microtubes and the extraction steps were repeated as previously described. The supernatant was added to matching microtubes and evaporated. The samples were reconstituted in 80 µl of 1:1 phosphate buffer (0.02M) : ACN, containing 1 mM EDTA and 0.1% mercaptoethanol by vortex-mixing for 2 minutes, centrifuged (18626 g, 5 min) and injected into Waters 2695 Separation Module HPLC system coupled with Waters 2475 Multi Gamma Fluorescence Detector (Waters Co., Milford, MA).

3.1.9 Chromatographic analysis: 50 µl of reconstituted APO samples were injected into BDS Hypersil Cyano (5 µm, 250 mm × 4.6 mm) column with Hypersil Cyano pre-column (5 µm, 4 mm × 10 mm) at flow rate of 1.2 ml/min and temperature of 30° C. Table 24 shows mobile phase linear gradient of ACN and phosphate buffer (in 1 L DDW: 2.42 g NaH₂PO₄*H₂O, 0.28 ml H₃PO₄, 45.45 mg EDTA; pH adjusted to 3.0 using NaOH and H₃PO₄.). Detection was done at excitation wavelength of 270 nm and emission 450 nm. Retention time for APO and benzo(a)pyrene were 7.1 and 17.1 min respectively. Linearity was shown for the two molecules between 1 - 1000 ng/ml with R²>0.997.

TABLE 24

HPLC analysis of in vivo results

| Time (min) | Acetonitrile (%) | Phosphate buffer (%) |
|---|---|---|
| 0 | 10 | 90 |
| 8 | 10 | 90 |
| 15 | 70 | 30 |
| 19 | 70 | 30 |
| 21 | 10 | 90 |
| 30 | 10 | 90 |

Mobile phase gradient for HPLC analysis of in-vivo results.

3.1.10 Statistical and pharmacokinetic analysis of in-vivo study: All values are expressed as mean ±standard error of the mean (SEM) or as otherwise specified. The concentration vs time in-vivo data was analyzed by a non-compartmental PK analysis using Phoenix WinNonlin 8.1 (Certara, USA). Following this analysis, PK parameters were obtained.

### 3.2 Results

### 3.2.1 Ex-Vivo Permeability Studies

FIG. 19 shows the comparison of Papp of APO and permeation markers MTP and ATN through excised porcine buccal mucosa from three intervention groups: (1) simulated saliva solution at pH 7.4, (2) at pH 5.9, and (3) at pH 7.4 with addition of 3.33%^{v}/ᵥ 1:1EtOH:PG. Papp values are shown in Table 25.

ATN Papp at pH 7.4 with and without the addition of 3.33% ^{v}/ᵥ 1:1EtOH:PG was significantly lower than of MTP or APO. In contrast, no significant difference was found between Papp of ATN, MTP or APO when tested from simulated saliva at pH 5.9. Under these conditions ATN Papp was higher compared to the value at pH 7.4 (although not statistically significant), while MTP and APO Papp were 2- and 6-fold lower, respectively (with statistical significance) than the corresponding value at pH 7.4. No statistical difference was found for ATN in the three intervention groups. Comparing MTP and APO in pH 7.4 with addition of 3.33%^{v}/ᵥ 1:1EtOH:PG and pH 7.4, it was found that the former causes statistically significant elevation in Papp values by 3- and 4-fold respectively.

FIG. 20 shows ATN, MTP and APO cumulative amount permeated through excised porcine buccal mucosa either from simulated saliva solution at pH 7.4 or from simulated saliva solution at pH 7.4 with the addition of 10%^{v}/ᵥ NLF. No difference between the two groups was observed for ATN profiles, showing slow but linear accumulation in the receiver compartment. In contrast, a completely different permeation profiles were obtained for APO and MTP. For both substances, the group without 10%^{v}/ᵥ NLF showed linear permeation after a lag-time of ~1 hour, matching the Fickian diffusion. On the other hand, the addition of 10%^{v}/ᵥ NLF resulted in a lag-time of 4 h for MTP and 3 h for APO. Due to limited vitality of the excised buccal tissue, the experiment was terminated before the linear permeation phases were obtained for MTP and APO. For APO, although at the end of the experiment the obtained slope was similar to the group without 10%^{v}/ᵥ NLF, the results were highly variable and unequivocal.

TABLE 25

Apparent permeability (Papp) values

| | Atenolol | Metoprolol | Apomorphine |
|---|---|---|---|
| pH 7.4 | 0.06 (0.06) | 2.48 (0.22) | 3.00 (0.26) |
| pH 5.9 | 0.24 (0.09) | 1.11 (0.25) | 0.51 (0.01) |
| 3.33%^{v}/ᵥ of 1:1 ethanol: propylene glycol solution | 0.19 (0.12) | 7.24 (0.97) | 11.63 (0.82) |

Apparent permeability (Papp) values (10⁻⁶ cm/sec) of atenolol, metoprolol and apomorphine through excised porcine buccal mucosa from simulated saliva at pH 7.4, pH 5.9, or pH 7.4 with addition of 3.33%^{v}/ᵥ of 1:1 ethanol : propylene glycol solution. Mean (±SD), n=3.

FIG. 21 shows APO Papp through excised porcine buccal mucosa from simulated saliva solution at pH 7.4 with 100-fold higher APO concentration of 9.6 mM in the donor compartment compared to standard test solution of 0.1 mM. The Papp values obtained for the two groups were practically the same, 3.13 (±0.34) * 10⁻⁶ cm/sec and 3.00 (±0.26) * 10⁻⁶ cm/sec respectively.

### 3.2.2 Simulation to Human Plasma Levels

Further experiments were performed to assess whether the APO obtained permeability rates are therapeutically relevant for treating PD. Applying Eq. 6 and Eq. 3 calculations of the theoretical APO Jss and Css levels in human plasma that would be obtained upon APO administration were performed. Initial concentration (Co) was 20 mg/ml which is the maximal aqueous APO solubility. Application area (A) was 50 cm² which corresponds to buccal area in humans. CL value from literature was in the range of 3-5 L/h/kg. APO Css from the three intervention groups for both CL value extremities in human weighting 70 kg was calculated. Results are shown in Table 26.

TABLE 26

Simulated APO flux through buccal mucosa and its plasma concentration

| | Papp obtained from ex-vivo study (10⁻⁶ cm/sec) | Jss calculated (mg/(cm²*h)) | Css calculated (ng/ml) |
|---|---|---|---|
| pH 7.4 | 3.00 | 0.22 | 51.5 - 31.0 |
| pH 5.9 | 0.51 | 0.04 | 8.5 - 5.0 |
| 1:1 ethanol: propylene glycol | 11.63 | 0.84 | 200.0 - 120.0 |

Simulated APO flux through buccal mucosa and its plasma concentration at plateau for 70 kg human after application of 20 mg/ml aqueous solution of apomorphine to buccal area of 50 cm². Papp - apparent permeability, Jss - flux, Css - plasma concentration at steady state

### 3.2.3 Intravenous Administration of APO to Rats

FIG. 22 shows APO plasma levels after IV administration of 2.0 mg/kg dose to rats. The obtained PK parameters were: half-life = 15.1 min, volume of distribution = 3.3 L/kg, and CL = 9.2 L/h/kg.

### 3.2.4 Simulation of Plasma Levels After Buccal Administration

To determine the ability to detect quantifiable APO plasma levels upon administration to rat buccal mucosa, simulation was performed using PK parameters after IV administration and Eq. 6 and Eq. 7: Co = 19 mg/ml, A = 0.2 cm², average rat weight 330 gr and Papp = 0.51*10⁻⁶ cm/sec. Papp through porcine buccal mucosa was selected (as lower Papp is of no clinical relevance). Experiments were performed at pH 5.9, as in this pH APO has maximum solubility and the permeability would be prolonged to simulate prolonged release. The calculated Css was 2.3 ng/ml, which is higher than the limit of quantification and should be detectable.

### 3.2.5 In-Vivo Permeability Study on Rats

APO solution was applied to the buccal mucosa of three anesthetized rats for 8 h. APO was not detected in the plasma at any time point. The APO concentration in the solution inside the delivery device attached to rats’ buccal tissue did not change at 8 h compared to initial concentration administered.

### 3.3 Conclusions

This work was undertaken to determine whether the permeability rate of APO through buccal mucosa is relevant to obtain therapeutic plasma levels to treat PD. Several APO permeability attenuating methods were studied to obtain the ability to control the rate of APO appearance in blood.

APO Papp through excised porcine mucosa was studied simultaneously with the permeability markers ATN and MTP at pH 7.4. ATN and MTP Papp were similar to the values reported in literature under the same conditions, suggesting that the system worked properly. The low ATN Papp suggested that the tissue was intact in all the experiments, and further that the paracellular diffusion rate through the buccal mucosa was ‘low’. As opposed to MTP Papp value which is the standard for ‘high’ passive transcellular diffusion. By comparing the obtained APO Papp value to ATN and MTP Papp, APO was relatively similar to MTP. Administration of 100-fold higher APO concentration resulted in the same Papp, suggesting that the permeability of APO occurs through a passive transcellular diffusion than active transporter mediated permeation. Further, 9.6 mM APO concentration equals to 3 mg/ml dosage, which is a clinically relevant dose of this drug for PD. It suggests that any results obtained ex-vivo with the standard test concentration 0.1 mM utilized in Ussing diffusion chamber might be relevant also for dosages utilized in clinical setup.

The next step of this work was to study the permeability attenuating methods to obtain the ability to control the APO delivery rate through the buccal mucosa. First method was to study the effect of pH on the permeability. Several works have shown that Papp of different molecules changes due to pH. pH of 5.9 is considered representative of oral pH. A pH higher than 7.4 is of low relevancy as APO is practically insoluble in aqueous solutions at those values. At pH of 5.9, APO Papp was significantly lower compared to pH 7.4. From the computed profile of APO charge vs pH, it was calculated that the electrical charge of the molecule decreases from pH ~6 to pKa 8.9 wherein the molecule becomes uncharged. Consequently, at pH close to pKa the molecule will have higher lipophilic properties and will be non-water soluble, while at a lower pH the molecule will be more soluble and less lipophilic. Thus, when comparing transcellular permeability through lipidic membranes of the buccal mucosal cells, the permeability is higher at pH 7.4 and lower at pH 5.9. The same phenomenon was obtained for MTP which pKa is 9.6. Slight statistically insignificant increase in ATN Papp was observed when pH changed from 7.4 to 5.9. Although pKa of ATN is 9.6, ATN permeates via paracellular route through the aqueous spaces due to its low log P of ~0.2. Therefore, increase in ATN Papp corresponds to the increase in its polarity.

By changing pH locally at the site of the delivery device administration, permeability rates of delivered drugs can be attenuated. Specifically for APO, a slower permeability rate can be obtained by reducing local pH to 5.9. This phenomenon can be applied for controlled delivery of APO by prolongating the absorption of the delivered dose for a longer period.

Addition of 3.33%^{v}/ᵥ 1:1EtOH:PG caused dramatic elevation of APO and MTP Papp. This could be explained by the fact that organic solvents enter ordered phospholipid bilayer of the buccal mucosal cells and increase its fluidity, thus enabling faster diffusion of lipophilic molecules. Only slight and not statistically significant elevation of ATN Papp was observed. As was previously shown, organic solvents can interfere with the order of lipids in the intercellular spaces and extract them. This will cause increase in aqueous space between the cells and enable more area for diffusion of hydrophilic molecules. Here, and in the group of pH 5.9 the inability to obtain statistical significance for ATN may be due to high variability of the results. This came at a cost of working with standard substance concentration in Ussing diffusion chamber of 0.1 mM and due to very low ATN permeability. Perhaps, statistical significance can be obtained with higher ATN concentration in the donor compartment.

The addition of organic solvents to the delivery system, in general, can increase permeability rates to achieve higher steady state concentrations in blood. However, the depletion of the depot in the device will be proportionally faster. In addition, the irritating effect of organic solvents to the mucosa should be taken into consideration, especially when administering a long-term device.

The addition of 10%^{v}/ᵥ NLF yielded surprising results. This formulation has previously shown to increase per oral bioavailability of different molecules and its constituents in separation were shown to increase permeability of different actives. In the present experiments, a very long lag-times of 3 h and 4 h was observed for APO and MTP respectively, after which the molecules began to accumulate in the receiver compartment. Due to limited vitality of the buccal tissue, the experiment had to be terminated before the straight line on the diagram showing accumulated amount vs time was reached (although for APO, the end slope became similar to the control group, without the addition of the formulation). There were not enough time points tested to obtain validity and the variability was too high to reach unequivocal conclusions. In contrast, no lag-time was observed for ATN, and the accumulation profile was similar to the control group, suggesting that the nanoparticles obtained after addition of NLF to the donor compartment could serve as a storage vehicle for the lipophilic molecules. The lipidic core of nanoparticles enables preferential dissolution of lipophilic APO and MTP in the particles vs water medium. As such, the test molecule become unavailable for permeation through the buccal mucosa. However, due to oil/water partition coefficient some molecules remain in the aqueous medium and subsequently reach and permeate the buccal tissue into the receiver compartment and begin to accumulate. For ATN, a hydrophilic molecule that does not have high affinity to lipidic core of nanoparticles, no lag-time was produced.

Therefore, it seems that when lipophilic substances are administered in the medium containing micelle forming agent or, as in this case, nanoparticles with lipid core, the permeability of the lipophilic substances is decreased or significantly delayed. Additional research should be done to better understand these processes and determine the behavior of lipophilic substances at these conditions. Nevertheless, these types of systems can be useful for developing controlled release through buccal mucosa and prolonged or delayed delivery systems.

Steady state APO plasma levels in 70 kg human were simulated using Papp results. In human subjects, the therapeutic window is approximately 1.5 - 10 ng/ml, which is within the therapeutic window for this drug. Therefore, it was concluded that buccal APO delivery has therapeutic relevance for treating PD as a safe and convenient substitute to subcutaneous infusions. Further, as porcine buccal mucosa is thicker than human mucosa, even higher APO plasma levels are anticipated in humans.

The strength of ex-vivo studies in diffusion chambers is in its high availability, low costs and convenient logistics. These factors enable large screening of permeability rates of different molecules through biological membranes as well as investigation of permeability attenuating techniques. Nevertheless, excised mucosa is not exactly as in the whole animal. In addition, highly limited volume of the receiver compartment is a serious pitfall compared to large volume of blood, lymph and tissues in the whole animal. Therefore, ex-vivo methods are prone to false results, false-positive due to technical issues, but more importantly and less predictably, false-negative due to factors mentioned. Thus, the obtained ex-vivo results should be accepted with caution and further in-vivo research is necessary. This is especially true considering lack of standardization of this kind of experiments with buccal mucosa. While no standardization is available, no good ex-vivo - in-vivo correlation can be obtained. With the amassing data in the field of oral mucosal delivery, steps towards standard experimental protocols should be undertaken.

This is also true regarding permeability markers use in this study. As was shown, the ‘cocktail’ approach of permeability markers can add power to the results, sheds new light and enables broader understanding of the mechanistic processes involved. Permeability of a substance of interest can be explained with more detail and compared to the co-administered permeability markers. While this approach is mandatory in ex-vivo studies of permeability through intestine, it is not undertaken in studies with oral mucosa. Defining standard permeability markers for ex-vivo research of permeability through oral mucosa is an obligatory step towards standardization of this field.

Investigating buccal APO permeability in-vivo was further investigated. APO permeability was studied from aqueous solution at pH 5.9 in-vivo by applying it to the buccal mucosa of anaesthetized rats. APO was not detected in rat plasma through 8 h. Simulation with PK parameters of APO IV administration to rats was used to project APO plasma levels, presuming that the permeability through rat mucosa is similar to porcine, which suggested that it would be higher than the limit of quantification. Thus, inability to detect them after buccal administration is not a technical issue. The fact that APO concentration in the solution administered did not change after 8 h further strengthens the conclusion that APO does not permeate through rat buccal mucosa, at least not to the extent needed for clinical relevance. As was noted, the rat buccal mucosa is very different from hominine, the main difference is that it is highly keratinized and consequently is considerably less permeable. Therefore, it can be concluded that rat is not an appropriate animal model for studying APO buccal mucosal administration and conventional animal model such as pig is more informative.

### Example 4 Prolonged Controlled Delivery of Drugs Through Oral Mucosa Cannabidiol Paradigm

It was hypothesized that the major part (if not all) CBD administered through the oral mucosa is washed from the mucosa by salivary flow and is subsequently ingested. There are two main reasons for this assumption: (1) inability of cannabinoids to permeate fast enough, or at all, through the oral mucosa and remaining on the surface; (2) their accumulation in the tissue and subsequent release back into the oral cavity. No data is available in literature on the permeability rates of CBD and THC through oral mucosa.

In the first part of the research, permeability rates of CBD were investigated ex-vivo using excised porcine buccal mucosa in Ussing diffusion chamber. The advantages of this model were discussed above. CBD permeability was investigated from simulated saliva as well as with the addition of 3.33%^{v}/ᵥ 1:1 ethanol:propylene glycol solution, mimicking the solvent used for cannabinoids’ delivery in Sativex®. 4% bovine serum albumin was added to the receiver side of Ussing diffusion chamber to simulate plasma protein content and enable to CBD binding and sink conditions with a driving force to enter the receiver side medium. Furthermore, a novel approach was adopted using a ‘cocktail’ with simultaneous co-administration of permeability ATN and MTP markers into the donor medium. The properties of these two molecules as well as the advantages of using them as standards of buccal mucosa delivery were previously discussed.

In the second part of the research, to further investigate the ability of CBD to permeate oral mucosa and reach blood circulation to produce detectable and clinically relevant plasma levels, in-vivo study in pigs was conducted. CBD was dissolved in a solution mimicking Sativex®. However, as opposed to short exposure in Sativex® spray application, CBD was applied for 8 h to enable slow absorption and to determine absorption kinetics into the blood circulation. The washing away by the salivary flow was prevented by introduction of water impermeable layer, which further prevented the escape of CBD solution from the site of administration into the oral cavity and contained it on the oral mucosa. Further, to better understand the mechanisms of CBD permeation through oral mucosa, a ‘cocktail’ approach was adopted by introducing theophylline (TPH) into the CBD solution. TPH has high permeability through biological membranes, but as opposed to CBD, it is hydrophilic with log P of ~0. Both molecules were co-administered to the mucosa simultaneously in the same donor solution. It was hypothesized that it would not accumulate in the tissue and would have different absorption pattern from CBD. The difference between the two test molecules was supposed to add mechanistic insight into the absorption process.

### 4.1 Materials and Methods

4.1.1 Tissue and system preparation for ex-vivo permeability studies: Ussing diffusion chamber (VCC MC6 EasyMount, Physiologic Instruments Inc., USA) system was used for assessment of CBD, MTP and ATN permeation rates through excised porcine buccal mucosa. Each chamber includes a heating block for temperature control and needle valves for adjustment of carbogen gas flow for oxygenation and gas lift stirring. Buccal tissues of female pigs, ~100 kg, were used in the experiments. Pigs were raised at Lahav ltd. Israel. Pigs were exposed to anesthetic agents, antibiotics, contrast agents and sacrificed by over exposure to KCl. There is no prior knowledge on interference of these agents with the experimental protocol. Immediately following euthanasia, buccal tissue was removed and placed in ice-cold modified Ringer’s buffer (pH 7.4). Buccal mucosa segments were trimmed to 800-1000 µm by surgical scissors and tweezers and mounted into Ussing diffusional chambers. The exposed tissue surface area was 0.5 cm² and buffered solutions volume in each cell was 3 ml. The system was preheated to 35 (±1)°C. Modified Ringer’s buffer containing 4% bovine serum albumin was added to the receiver side and simulated saliva buffer to the donor side. The tissue oxygenation and the solution mixing were performed by bubbling with carbogen gas. The system was equilibrated for 30 min followed by replacing the solutions and adding 20 µl of the test solutions to the donor side of the chamber to commence the experiment.

4.1.2 Preparations of test solution and buffers: The simulated saliva buffer was prepared by mixing 105 mg NaHCO₃, 65.8 mg KH₂PO₄, 133.5 mg K₂HPO₄, 36.8 mg CaCl₂*2H₂O and 55.9 mg KCl in 250 ml DDW. pH was adjusted to 7.4 with NaOH and HCL. Modified Ringer’s buffer solution with 4% bovine serum albumin was prepared by mixing 6.54 g NaCl, 0.18 g CaCl₂^{∗}2H₂O, 0.37 g KCL, 0.24 g MgCl₂*6H₂O, 2.1 g NaHCO₃, 0.23 g Na₂HPO₄, 0.05 g NaH₂PO₄*H₂0, 1.44 g D-glucose, 0.36 g mannitol and 40 g bovine serum albumin in 1000 ml DDW. pH was adjusted to 7.4 with NaOH and HCl. Test solution contained 16.2 mg ATN, 20.6 mg MTP and 18.9 mg CBD which were dissolved in 4 ml methanol solution followed by vortex-mixing for two minutes. Solutions were freshly prepared on the days of the experiments. When testing the effect of organic solvents, 100 µl of the simulated saliva in the donor chamber were replaced by 100 µl of 1:1 ethanol:propylene glycol solution.

4.1.3 Experimental protocol: 200 µl samples were taken from receiver chamber at the beginning of the experiments, at half an hour and every hour for 6 hours. 200 µl of fresh modified Ringer’s buffer solution with 4% bovine serum albumin were added each time to receiver chamber. The dilution of the concentration of tested substances in the receiver chamber was accounted for. All samples were stored at -20° C. pending analysis.

4.1.4 Chromatographic analysis of ex-vivo study: 10 µl of THC 100 µg/ml ethanolic solution as an internal standard was added to 200 µl thawed samples and vortex-mixed for 2 min. 800 µl of methanol was added, vortex-mixed for 5 min and centrifuged for 10 minutes at 18626 g. Supernatant was filtered through 0.20 µm Millipore Millex®-FG PTFE filters. Additional 500 µl of pure methanol was transferred through the filters and added to the supernatant. Supernatant was evaporated to dryness under vacuum and reconstituted in 80 µl of 0.1% formic acid containing 3:2 acetonitrile:DDW. 10 µl of the supernatant was injected into the LC-MS system comprised of Waters 2695 Separation Module HPLC system, and Waters Micro-mass ZQ mass spectrometer (Waters Co., Milford, MA). The analysis conditions were as follows: XTerra MS (Waters Inc.) C18 Column (3.5 µm, 2.1 mm x 100 mm), temperature = 45° C., flow rate = 0.2 ml/min and mobile phase with a linear gradient as detailed in Table 27.

TABLE 27

Chromatographic analysis

| Time (min) | Acetonitrile + 0.1% formic acid (%) | 0.1% formic acid |
|---|---|---|
| 0 | 5 | 95 |
| 10 | 80 | 20 |
| 20 | 80 | 20 |
| 23 | 5 | 95 |
| 40 | 5 | 95 |

Mobile phase gradient for LC-MS analysis of Ussing chamber permeability studies.

Retention times were as follows: ATN 4.2 min, MTP 8.9 min, CBD 16.2 min and THC 17.9 min. Detection was done at [M+H]+ with m/z: ATN 267.3, MTP 268.3, and CBD and THC 315.2; at desolvation temperature of 350° C. and source temperature of 110° C., ion spray voltage of 3 KV, cone voltage of 22 V and extractor voltage of 3 V. Linearity was found for all molecules between 10-1000 ng/ml with R²>0.997.

4.1.5 Mucoadhesive ‘shell’ preparation: Using mortar and pestle several grains of methyl red were triturated with 1500 mg mannitol to fine homogenous pink powder. A cylindrical tablet was prepared by direct compression in a 13 mm flat-faced punch and die set, applying a pressure of 2 ton/cm² for 30 sec. Obtained tablet was placed in the middle of 20 mm flat-faced punch and die set. A homogenous mix of 566.7 mg hydroxypropyl cellulose and 1133.3 mg Carbopol 934 with 8.5 mg Mg stearate (lightly mixed) were poured over the tablet into the die. 8 mm thick cylindrical tablet was prepared by direct compression of 3 ton/cm² for 30 sec. The mannitol core (easily differentiated by a pink color) was scraped out from the hydroxypropyl cellulose - Carbopol 934 shell by a spatula. The inner side of the shell was covered with a parafilm® M. A 0.82 mm hole through the flat side was punched by a 21-gauge needle.

4.1.6 In-vivo experiment protocol: Domestic female pigs (sus scrofa domesticus) of 35 - 40 kg body weight were obtained from Lahav ltd. Israel. After arrival to the unit for experimental surgery at Hadassah Ein-Kerem medical center (Jerusalem, Israel) they were allowed to acclimate during a period of a week with free access to food and water, and were under veterinary surveillance. Pigs were fasted overnight before the experiments. On the morning of the experiment pigs were anesthetized by xylazine 1 mg/kg, midazolam 0.04 mg/kg, ketamine 5 mg/kg and propofol 4 mg/kg. They were positioned on their back. Endotracheal tube was inserted, and pigs were respired with oxygen. The anesthesia was maintained by 1.9% isoflurane at 1 L/min rate. Catheter was inserted into the ear vein through which physiological saline was administered. Pigs received amoxicillin 15 mg/kg antibiotic and tolfenamic acid 2 mg/kg analgesic. An intra-jugular catheter was inserted for blood sampling. Pigs were constantly monitored for their respiration rate, rectal temperature, O₂ saturation, heart rate and blood pressure. Cardiovascular parameters were obtained by blood pressure monitor with porcine adapted front leg cuff. In three pigs, with mean weight of 43 kg (SEM ±2.1), the Inner side of the lower lip was gently cleaned with a gauze. A mucoadhesive “shell” was adhered to it and pressed with finger for 1 min, to ensure full adhesion. 600 µL of 1:1 ethanol:propylene glycol solution containing 18 mg CBD and 4.8 mg TPH was administered by a 23-gauge needle through the hole in the flat side of the mucoadhesive “shell” into the cavity inside the “shell”, thus reaching the surface of the mucosa. Visual inspection approved that the mucoadhesive “shell” was fully adhered to the mucosa and there were no leakages. 10 ml blood samples were taken through the jugular catheter into heparinized tubes 10 min before the administration of drug solution into the mucoadhesive “shell” adhered to pig and after administration at 0.25, 0.5, 0.75, 1, 1.33, 1.67, 2, 3, 4, 5, 6, 7, 8, 8.33, 8.67, 9, 9.5, 10, 10.5, 11, 11.5 and 12 hours. The blood was centrifuged immediately after the sampling at 2540 g for 10 min. Plasma was separated and stored at -80° C. pending analysis. At 8 hours mucoadhesive “shell” was removed, the mucosa was carefully cleaned with a gauze, so that no CBD and TPH will remain on its surface. At 12 hours pigs were euthanized by injection of overdose of KCl.

4.1.7 CBD extraction method from plasma: 20 µL of THC dissolved in acetonitrile (1 µg/ml) as an internal standard and 2 ml of acetonitrile were added to 1.5 ml thawed plasma samples and vortex-mixed for 5 min. 5 ml of hexane were added, followed by vortex-mixing for 5 min and centrifugation at 2540 g for 7 min. Supernatant was transferred to clean tubes. 5 ml of hexane were added to the remaining aqueous phase followed by vortex-mixing for 5 min and centrifugation at 2540 g for 7 min. Supernatant was added to the previously obtained one. The twice collected supernatant was evaporated to dryness under vacuum at 40° C. The samples were reconstituted in 100 µl of 1:4 acetonitrile : DDW solution and injected into LC-MS-MS system.

4.1.8 TPH extraction method from plasma: 10 µl metoprolol methanolic solution (100 ng/ml) as an internal standard was added to 1.5 ml thawed plasma samples and vortex-mixed for 1 min. 350 µl Na₂HPO₄ (0.2 M) were added, followed by vortex-mixing for 2 min. 6 ml of diethyl ether was added followed by vortex-mixing for 5 min and centrifugation at 2540 g for 5 min. Supernatant was transferred to clean tubes and evaporated to dryness under vacuum. The samples were reconstituted in 100 µl 1:4 MeOH:DDW solution containing 0.01%^{v}/ᵥ formic acid and injected into LC-MS-MS.

4.1.9 Chromatographic analysis of CBD from in-vivo study: The chromatography was performed using a Thermo Scientific (San Jose, CA, USA) UHPLC System, which includes Dionex Pump with degasser module and an Accela Autosampler. The chromatographic separations were performed on a Kinetex™ (Phenomenex, CA, USA) column (EVO C18, 2.6 µm, 100 Å, 50 × 2.1 mm), protected by a SecurityGuard™ (Phenomenex, CA, USA) ULTRA cartridge (C18, 4 × 2.1 mm). The injection volume was 25 µl, the oven temperature was maintained at 40° C. and the autosampler tray temperature was maintained at 4° C. The chromatographic separation was achieved using a linear gradient program Table 28 at a flow of 0.3 ml/min over a total run time of 9 min.

TABLE 28

Chromatographic analysis

| Time (min) | Solvent A (%) | Solvent B (%) |
|---|---|---|
| 0.0 | 40 | 60 |
| 4.0 | 5 | 95 |
| 6.0 | 5 | 95 |
| 6.3 | 40 | 60 |
| 9.0 | 40 | 60 |

Gradient program: Solvent A is 0.01%^{v}/ᵥ formic acid in DDW and solvent B is acetonitrile.

The first 0.9 min and last 2.0 min of the column effluent were diverted to waste. Methanol was used for washing the needle prior to each injection cycle. All samples were analyzed in duplicates. CBD and THC were detected by a TSQ Quantum Access Max mass spectrometer in positive ion mode using electron spray ionization (ESI) and multiple reaction monitoring (MRM) mode of acquisition. The high-purity nitrogen gas (15 L/min), used as sheath and auxiliary gases, was generated using a Parker nitrogen generator (Parker Hannifin ltd., Gateshead, Tyne and Wear). 99.999% pure argon (Moshalion,) was used as a collision gas (1.5 mTorr). Transitions of the analytes are shown in Table 28. The molecular ion of the compounds [M+H]⁺ was selected in the first mass analyzer and fragmented in the collision cell followed by detection of the products of fragmentation in the second mass analyzer. The spray voltage, sheath and auxiliary gas were set at 5000 V, 30 and 60 (arbitrary units), respectively. The tube lens and the capillary transfer tube temperature were set at 91 V and 220° C., respectively. The vaporizer temperature within the H-ESI source was 450° C. The scan time was 50 msec, scan width 0.1 m/z, Q1 and Q3 peak width of 0.7 Da. The collision energies for the monitored transitions are given in Table 29. The dwell time was 30 msec.

TABLE 29

Transitions analysis

| Name | Precursor (m/z) | Product (m/z) | CE (eV) | Rt (min) |
|---|---|---|---|---|
| CBD | 315 | 193 | 20 | 2.6 |
| | | 123 | 32 | |
| THC | 315 | 193 | 20 | 3.5 |
| | | 123 | 32 | |

Multiple reaction monitoring transitions and parameters for the analytes in positive ion mode. While the first transition for each analyte was the quantifier ion, the second transition was the qualifier ion. m/z: mass to charge ratio; CE: collision energy; eV: electronvolts; Rt: retention time.

Data acquisition and analysis were performed with Analyst 1.6.2 software (Sciex). Linearity curve (0.1 - 3.333 ng/ml, R²>0.997) was performed before every batch of samples and used for quantification.

4.1.10 Chromatographic analysis of TPH: The chromatography was performed using a Shimadzu (Japan) UHPLC System, series Nexera, consisting of a Shimadzu CBM-20A LITE controller, two Shimadzu LC-30AD pumps, including a Shimadzu Prominence DGU-20A5R degasser, a Shimadzu SIL-30AC autosampler and a Shimadzu CTO-20AC column oven. The chromatographic separations were performed on a Kinetex™ (Phenomenex, CA, USA) column (EVO C18, 2.6 µm, 100 Å, 100 × 2.1 mm), protected by a SecurityGuard™ (Phenomenex, CA, USA) ULTRA cartridge (C18, 4 × 2 mm). The injection volume was 5 µL, the oven temperature was maintained at 40° C. and the autosampler tray temperature was maintained at 4° C. The chromatographic separation was achieved using a linear gradient program Table 30 at a flow of 0.3 ml/min over a total run time of 18 min.

TABLE 30

Chromatographic analysis

| Time (min) | Solvent A (%) | Solvent B (%) |
|---|---|---|
| 0.0 | 95 | 5 |
| 9.0 | 65 | 35 |
| 10.0 | 2 | 98 |
| 14.0 | 2 | 98 |
| 14.5 | 95 | 5 |
| 18.5 | 95 | 5 |

Gradient program: Solvent A is 0.01%^{v}/ᵥ formic acid in DDW and solvent B is MeOH.

The first 1.3 min and last 8.0 min of the column effluent were diverted to waste. A 1:1 mixture of MeOH:DDW was used for washing the needle prior to each injection cycle. All samples were analyzed in duplicates. TPH and metoprolol were detected by a Sciex (MA, USA) Triple Quad™ 5500 mass spectrometer in positive ion mode using electrospray ionization (ESI) and multiple reaction monitoring mode of acquisition. Air was produced (SF 2 FF compressor, Atlas Copco, Belgium) and purified using an NM20Z nitrogen generator (Peak Scientific, Scotland). Purified air was used as source and exhaust gases and purified nitrogen as curtain and collision gases. A receiver was placed between the compressor and the nitrogen generator for a large and stable supply of air. The transitions of the analytes are shown in Table 31. The molecular ion of the compounds [M+H]⁺ was selected in the first mass analyzer and fragmented in the collision cell followed by detection of the products of fragmentation in the second mass analyzer. The TurboIonspray® probe temperature was set at 600° C. with the ion spray voltage at 5.5 KV. The curtain gas was set at 30.0 psi. The nebulizer and turbo heater gases were set to 40 psi and the collision gas was set to 8 psi. The entrance potential was set at 10 V. The dwell time was 30 msec.

TABLE 31

Transitions analysis

| Name | Precursor (m/z) | Product (m/z) | CE (eV) | Rt (min) |
|---|---|---|---|---|
| TPH | 180.6 | 124.1 | 25 | 4.5 |
| | | 96.1 | 35 | |
| Metoprolol | 268.0 | 116.0 | 27 | 8.4 |
| | | 98.2 | 25 | |

Multiple reaction monitoring transitions and parameters for the analytes in positive ion mode. While the first transition for each analyte was the quantifier ion, the second transition was the qualifier ion. m/z: mass to charge ratio; CE: collision energy; eV: electronvolts; Rt: retention time.

Data acquisition and analysis were performed with Xcalibur program (ThermoScientific,). Linearity curve (0.033 - 3.333 ng/ml, R²>0.997) was performed before every batch of samples and used for quantification.

4.1.11 Statistical and PK analysis: Statistical analysis of ex-vivo study was performed by two-way factorial ANOVA with Tuckey post-hoc analysis simultaneously over all the results. Statistical significance was stated for p<0.05. All values are expressed as mean ±standard deviation (SD) or as otherwise specified. For in-vivo experiments all values are expressed as mean ±standard error of the mean (SEM), or as otherwise specified. The concentration vs time in-vivo data was analyzed by a non-compartmental PK analysis using Phoenix WinNonlin 8.1 (Certara). Following this analysis, PK parameters were obtained.

4.1.12 Equations utilized: Apparent permeability (Papp) is calculated by Eq. 8:

$$\text{Papp} \text{=} \left(\text{dQ} / \text{dt}\right) / \left(\text{A*Co}\right) \qquad \text{(8)}$$

where dQ/dt is the amount of test substance permeated over time, A is the permeation area of buccal mucosa inside the Ussing chamber of 0.5 cm², and Co is the initial concentration of the test substance in the donor chamber at the beginning of the experiment.

The flux (Jss) of the test substance through the mucosa is calculated by the Eq. 9:

$$\text{Jss} \text{=} \left(\text{dQ} / \text{dt}\right) / \text{A} \qquad \text{(9)}$$

Simulation of blood levels at steady state (Css) of the substances if administered in-vivo is calculated by Eq. 10:

$$\text{Css} \text{=} \text{Jss} / \text{CL} \qquad \text{(10)}$$

where CL is the pharmacokinetic parameter of total clearance of the tested substance in-vivo.

### 4.2 Results

### 4.2.1 Ex-Vivo Studies

Table 32 shows the results from ex-vivo studies in Ussing diffusion chamber. CBD was not detected in the receiver side medium. ATN Papp in simulated saliva was significantly lower than for MTP (p<0.05), and was not detected in the receiver side medium when administered with the addition of 3.33%^{v}/ᵥ 1:1 EtOH:PG. MTP Papp was 10-fold higher (p<0.05) when administered with addition of 3.33%^{v}/ᵥ 1:1EtOH:PG compared to the administered only in simulated saliva.

TABLE 32

Apparent permeability (Papp) values

| | Atenolol | Metoprolol | Cannabidiol |
|---|---|---|---|
| pH 7.4 | 0.09 (0.002) | 1.02 (0.05) | ND |
| 3.33%^{v}/ᵥ of 1:1 ethanol: propylene glycol solution | ND | 9.89 (2.29) | ND |

Apparent permeability (Papp) values (10⁻⁶ cm/sec) of atenolol, metoprolol and cannabidiol through excised porcine buccal mucosa from simulated saliva with and without addition of 3.33%^{v}/ᵥ of 1:1 ethanol : propylene glycol solution. Mean (±SD), n=3. ND = not detected. p<0.05 between all the reported values (two-way ANOVA with Tukey post hoc analysis).

### 4.2.2 In-Vivo Study

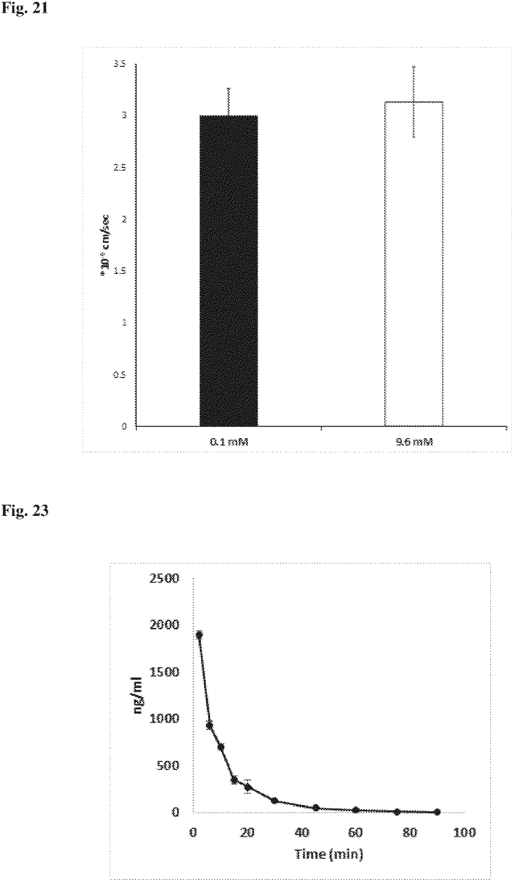

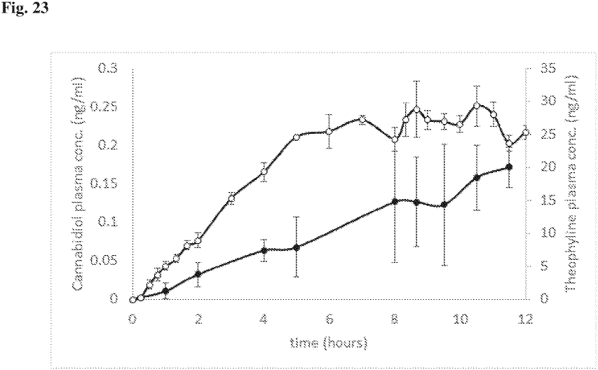

FIG. 23 shows a plot of CBD and TPH concentration in plasma vs time following administration of ~0.42 mg/kg dose of 30.0 mg/ml CBD and ~0.11 mg/kg dose of 8.0 mg/ml TPH in 600 µl of 1:1 ethanol:propylene glycol solution with exposed area for absorption of 1.58 cm² to three female pigs with average weight of 43 kg. For CBD, a slow elevation in plasma levels started immediately following administration and continued to rise at constant rate through 8 h until removal of the solution from the mucosa. CBD levels continued to elevate with practically the same rate through 12 h until termination of the experiment. For TPH, a much faster elevation in plasma levels was observed soon after administration of the solution to the mucosa. The levels reached plateau after 5 h, which was 300-fold higher (considering the molar dose administered) than the highest plasma level of CBD. The TPH plateau levels remained relatively stable through 8 h until removal of the solution from the mucosa, as well as afterwards through 12 h until termination of the experiment.

### 4.2.3 Safety Study

Table 33 shows vital parameters monitored following CBD and TPH administrations to the mucosa. Parameters were compared to normal as described in literature. The table shows median with the lowest and highest values detected in all three pigs. All parameters were at normal range, except diastolic pressure which was lower than normal. Nevertheless, it was always constant in all three pigs.

TABLE 33

Apparent permeability (Papp) values

| | Rectal temp (°C) | Heart rate (beats/min) | Systolic blood press (mmHg) | Diastolic blood press (mmHg) | O₂ satur (%) | Respiration rate (min⁻¹) |
|---|---|---|---|---|---|---|
| Obtained | 37.4 (36.8 - 38.8) | 98 (79 - 123) | 77 (68 - 87) | 37 (29 - 46) | 99 (90 - 100) | 10 (10-13) |
| Literature | 36.7 - 39.2 | 60 - 140 | 73 - 230 | 52 - 165 | ≥95 | 6-20 |

Monitored vital parameters in pigs following administration of ∼0.42 mg/kg dose of 30.0 mg/ml CBD and ∼0.11 mg/kg dose of 8.0 mg/ml of TPH dissolved in 1:1 ethanol:propylene glycol solution. Presented as a median with lowest and highest values detected throughout all three pigs, and compared to reported in literature.

### 4.3 Conclusions

The permeability rate of CBD through oral mucosa was investigated using freshly excised porcine buccal mucosa in Ussing diffusion chamber. No quantifiable levels of CBD that permeated through the mucosa were detected. Several reasons may be the cause for this result. As was shown previously by number of works, lipophilic molecules tend to accumulate inside the oral mucosal tissue. Thus, the molecules are expected to remain inside the tissue and only minimal quantity would be released into the solution in the receiver side, obeying the partitioning rule between the lipidic and aqueous media. With log P of CBD being ~6, it is reasonable to assume that this explains the current case.

A possible cause preventing CBD from reaching the receiver side might have been adsorption to the Ussing chamber walls. Another possibility is potential CBD precipitation in the aqueous buffer at the donor side. However, even when precipitated, it can be assumed that a part of CBD will remain solubilized and will be available for permeation through the tissue. With every molecule leaving the solution to enter the tissue, another molecule will dissolve from the precipitate thereby producing a constant concentration in the solution. The aqueous solubility of CBD is ~12 µg/ml or ~0.04 mM, which is close to the standard concentration utilized in the Ussing diffusion. It is far greater than the limit of quantitation by. Thus, it seems that precipitation was not the reason for lack of detection of CBD.

Further, the ATN and MTP permeability markers co-administered with CBD permeated the mucosa with quantifiable levels on the receiver side. Their Papp values corresponded to those previously reported in literature. This finding suggested that the system worked properly and inability to find CBD on the receiver side was due to its physicochemical properties.

To enhance the poor mucosal permeability of CBD, 3.33%^{v}/ᵥ 1:1 ethanol:propylene glycol solution was added to the donor medium. While mimicking Sativex® vehicle, it was previously shown that these constituents can increase the permeability of different substances through oral mucosa. Indeed, MTP Papp was increased 10-fold, which can be due to organic solvents entering cell membrane in the mucosa and elevating its fluidity. While transcellular marker MTP was highly influenced by the addition of organic solvents, ATN was not. Apparently, the paracellular pathway which utilizes the aqueous intercellular gaps was not influenced by the addition of organic solvents. The inability to detect ATN with addition of organic solvents was probably due to technical reasons related to quantitation limits by analytical instruments, which can be seen from a very low Papp obtained without the addition of ethanol:propylene glycol. Nevertheless, although MTP Papp was increased with addition of organic solvents, it did not modify the permeability of CBD, at least not to extent that could be detected on the receiver side. As CBD is highly lipophilic, it is reasonable to assume that it traverses the membrane by transcellular route. Yet, as can be seen by the results, addition of organic solvents was not enough for enhance its passage. If inability to detect CBD on the receiver side was due to its high accumulation in the tissue, it is consistent with the inability of organic solvents to cause its release into the receiver side.

The option to test the permeability of CBD via the relevant live tissue is of great importance in this certain investigation as it enables to isolate a part of the CBD absorption process and learn about the permeability properties in this specific part. The differences between the physicochemical properties of CBD and the ATN and MTP permeability markers were also reflected in the permeability properties. It is understood that a highly lipophilic drug undergoes a different process than a less lipophilic molecule administered under similar conditions. There are physiologic/pharmaceutic differences in the permeability of agents via excised porcine buccal tissue as opposed to the situation in-vivo. In in-vivo, there is a constant blood and lymph supply, and the diffusion passage is shorter. Therefore, studying the permeability process in-vivo is highly important.

To further investigate the ability of CBD to permeate oral mucosa and reach the blood circulation to produce detectable and clinically relevant plasma in-vivo, pigs received Sativex® mimicking solution containing clinically relevant daily dose of CBD. To retain the solution on the mucosa for prolonged period of time, it was contained inside a mucoadhesive ‘shell’. The ‘shell’ enabled access of the solution to the mucosa but was impermeable for the solution to leave the administration site and for saliva to enter and dilute or wash out the solution. Following the application of the solution, CBD levels in plasma increased at constant rate and remained elevated until removal of the solution after 8 h. Interestingly, following removal of the solution from the administration site, the increase in blood levels continued with relatively the same kinetics. While elevation in blood levels was rather slow, it did reach clinically relevant plasma concentrations compared to those obtained for Sativex® in clinical setup.

The ability to obtain CBD plasma levels proves that it permeates oral mucosa. To be able to calculate CBD flux through the mucosa, the parameter of plasma concentrations at steady state is required (Eq. 10). Css was not reached during the exposure time. The inability to reach plasma levels steady state can be explained by inability to reach equilibrium between the incoming CBD rate which permeated the tissue (Jss) and the elimination rate (CL). While clearance is constant, theoretically starting from the first molecule arriving to the blood stream, the incoming rate requires time (referred to as lag-time) to reach constancy. During this time (and in case the incoming flux is faster than the elimination rate), increase in plasma levels can be observed. As the steady state was not obtained through 8 h in which the CBD solution was present on the mucosa, wherein continuous increase in plasma was detected, one can assume that the incoming rate did not reach equilibrium. This means that plasma levels were obtained by the ‘avant-garde’ of CBD dose, which slowly permeated the mucosa with the main quantity remaining in the tissue. This tissue accumulation further explains CBD plasma levels profile following removal of the solution. Upon removal, no additional CBD enters the mucosa, the already accumulated amount continues to be released into blood stream, which subsequently causes continued increase in CBD plasma levels at the same rate as before the removal. Accumulation of CBD in the tissue and its slow release into aqueous medium can be explained by its very high log P of ~6. The explanation is supported by several other works with different molecules. In addition, high systemic tissue accumulation following IV administration is evident by its steady state volume of distribution in humans, which is as high as 30 L/kg.

To further validate the results of the study, a novel cocktail approach was adopted using co-administering in CBD and TPH, TPH is a hydrophilic with log P ~0. The plateau in TPH plasma levels was reached after relatively long lag-time of 5 h, suggesting slow CL of TPH (0.017-0.04 L/h/kg) that prevents reaching fast equilibrium with high incoming TPH rate due to high membrane permeability. Considering the molar dose administered to the mucosa, TPH steady state levels are 300-fold higher than the highest reached by CBD at the last time point of 12 h. These high TPH plasma levels can be explained both, by slower CL (~50-fold slower than CBD) and by high rate of entering the blood stream following fast permeation through the mucosa. As opposed to CBD, apparently the hydrophilic TPH does not accumulate in the tissue but enters the blood circulation rapidly following its penetration into the tissue from the delivery medium. Further, following removal of the solution from the mucosa, the plasma TPH levels remained almost at plateau for additional 4 h, suggesting on one hand, a low TPH CL that did not lead to the reduction of plasma levels, and on the other hand, an impediment of TPH entering the tissue. As no plasma elevation is obtained, the logical conclusion is that no more TPH is entering the blood stream. This is stands in contrast to the results with CBD. TPH, being hydrophilic, apparently does not accumulate in the tissue, and thus, with removal of the solution there is no more source of TPH to deliver to the circulation.

Results presented in this work show that CBD can permeate the oral mucosa. However, due to its slow permeability rate, the exposure time needs to be longer. In contrast with the Sativex® spray, the exposure is rather short due to washout by the salivary flow. Further with Sativex®, partial amounts of CBD that manage to enter the oral mucosa are prone to backwards release into the oral cavity and recurring washout by the saliva. The present method of concealing the delivered drug solution inside a mucoadhesive ‘shell’ reduces the washout effect and enables prolonged delivery of CBD. This method and its clinical applicability to treatments with CBD, and other cannabinoids, should be further investigated, especially in view of current lack of any controlled delivery systems for cannabinoids in the therapeutic arsenal. When developing a controlled release tool for systemic administration of cannabinoids through oral mucosa, and CBD in particular, one should take notice of the specific absorption profile of CBD, its very slow permeability rate through the oral mucosa and its high accumulation in the mucosal tissue with its subsequent prolonged sustained release.

Oral mucosa presents a considerable and distinct barrier with subsequent unusual absorption profile. When developing controlled release delivery device this absorption profile needs to be taken into consideration, and the device should be adapted accordingly.

### ADDITIIONAL EMBODIMENTS

In one of its main aspects the invention provides a bio-adhesive solid sticker for attaching to tissues within the oral cavity comprising of at least one poly(carboxylic acid) and an alcoholic small molecule that provides the sticker flexibility.

In certain embodiments the sticker of the invention is a single-sided adhesive.

In other embodiments the sticker of the invention is a double-sided adhesive.

In certain embodiments the sticker of the invention has different roughness on each side.

In certain embodiments the sticker of the invention releases different agents from each side.

In numerous embodiments the sticker of the invention comprises a combination of at least one polyol and at least one poly(carboxylic acid).

In further embodiments the at least one polyol is selected from the group consisting of: hydroxy propyl cellulose, hydroxypropyl methyl cellulose and their corresponding amylose derivatives, polyvinyl alcohol, natural gums, and chitosan.

In other embodiments the at least one polyacrylic acid is selected from the group consisting of crosslinked poly acrylic acid and methacrylic acid and their copolymers, alginates, hyaluronic acid, carboxymethyl cellulose and their mixtures.

In certain embodiments the sticker of the invention comprises a crosslinked poly(acrylic acid) and hydroxypropyl cellulose.

In certain embodiments the relative content (% w/w) of the crosslinked poly(acrylic acid) is from 100 to 40%

In numerous embodiments the sticker of the invention further comprises at least one edible alcohol.

In certain embodiments the edible alcohol is selected from the group consisting of ethanol, isopropanol, glycerol, monoglycedires, ethyl lactate, polyethylene glycols, and tributyl citrate.

In numerous embodiments the ratio of the edible alcohol to the total weight is between 1% to 20% (w/w).

In numerous embodiments the sticker of the invention further comprises a releasable active ingredient present in or attached to the sticker.

In certain embodiments the active ingredient is selected from the group consisting of analgesics, anesthetics, antiseptics, antibacterial agents, antiviral agents, disinfectants, herbal extracts, anti-halitosis agents, anti-inflammatory agents, opioids, and antidepressants.

In certain embodiments the releasable active ingredient or agent is a cannabinoid.

In further embodiments the cannabinoid is CBD and/or THC.

In other embodiments the active agent is a biological active agent, including peptide, protein, probiotic or antibiotic bacteria, oligonucleotide, and polynucleotide.

In certain embodiments the sticker of the invention is double-sided for attaching a desired element into the oral cavity.

In certain embodiments the desired element is an active agent eluting element including agents loaded in nano or microparticles that comprise active agents.

In numerous embodiments the active agents are selected from the group consisting of drugs treating oral disorders and drugs intended for systemic delivery.

In other embodiments the active agents are selected from the group consisting of small and peptide drugs, herbal extracts, homeopathic agents and combination thereof.

In numerous embodiments the active agents are selected from the group consisting of analgesics, anesthetics, anti-inflammatory agents, antiseptics, antibacterial agents, antiviral agents, disinfectants, herbal extracts, anti-halitosis agents, anti-inflammatory agents, opioids, cardiovascular drugs, caffeine, caffeine, nicotine, stimulating agents and antidepressants.

In certain embodiments the active agent is at least one cannabinoid.

In further embodiments the cannabinoid is CBD and or THC.

In numerous embodiments the sticker of the invention can be placed on any surface or surfaces within the oral cavity.

In certain embodiments the sticker of the invention can be placed locally onto or around ulcers for treating ulcers.

In numerous embodiments the sticker of the invention can have any geometric shape, flat shape, ring shape, smooth or rough in one or both sides, single composition of different compositions throughout the sticker.

It is another objective of the invention to provide a formulation for use in a prolonged buccal delivery device, the formulation comprising at least one water-insoluble or hydrophobic or lipophilic active agent, at least one dispersant, at least one lipid component and at least one amphiphilic solvent.

In numerous embodiments the formulation of the invention is characterized in that upon dispersion in an aqueous media the formulation forms a dispersion of nanodroplets or nanovehicles comprising the at least one water-insoluble or hydrophobic or lipophilic active agent.

In certain embodiments the formulation of the invention is a pro-nanodispersion system (PNS).

In certain embodiments the formulation of the invention is characterized by a size of nanodroplets or nanovehicles in the range between about 1 nm and about 1,000 nm.

In numerous embodiments the formulation is characterized in that the solubility of the at least one water-insoluble or hydrophobic or lipophilic active agent is lower or equal to 10 mg/ml in a water of neutral pH.

In certain embodiments the at least one water-insoluble or hydrophobic or lipophilic active agent comprised in the formulation is selected from cannabinoids, curcumin, apomorphine, amphotericin B, cyclosporine and rapamycin.

In further embodiments the at least one water-insoluble or hydrophobic or lipophilic active agent comprised in the formulation is apomorphine.

In further embodiments the at least one water-insoluble or hydrophobic or lipophilic active agent is a cannabinoid having affinity to CB1 or CB2 receptors.

In numerous embodiments the cannabinoid comprised in the formulation of the invention is selected from (CBD), delta-9-tetrahydrocannabinol (THC), cannabichromene (CBC), cannabichromenic acid (CBCA), cannabichromevarin (CBCV), cannabichromevarinic acid (CBCVA), cannabicyclol (CBL), cannabicyclolic acid (CBLA), cannabicyclovarin (CBLV), cannabidiol monomethylether (CBDM), cannabidiolic acid (CBDA), cannabidiorcol (CBD-C1), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), cannabinodiol (CBND), cannabinodivarin (CBVD), cannabinol (CBN), cannabinol methylether (CBNM), cannabivarin (CBV), cannabitriol (CBT), cannabitriolvarin (CBTV), delta-8-tetrahydrocannabinol (Δ8-THC), delta-8-tetrahydrocannabinolic acid (Δ8-THCA), delta-9-tetrahydrocannabivarin (THCV), delta-9-tetrahydrocannabivarinic acid (THCVA) and cannbicitran (CBT).

In numerous embodiments the at least one dispersant comprised in the formulation of the invention is selected from Tween, Span, phospholipids, polyethylene glycol (PEG), PEG-PPG block copolymers, PEG conjugated fatty chain and PEGilated hydrogenated castor oil.

In other embodiments the at least one dispersant is cremophor H40.

In numerous embodiments the at least one lipid component comprised in the formulation of the invention is selected from mineral oils and fatty acid esters.

In further embodiments the mineral oils and fatty acids are selected from liquid or solid mono-, di- and triglycerides and waxes.

In numerous embodiments the amphiphilic solvent comprised in the formulation of the invention is an organic solvent miscible in water or an aqueous media.

In certain embodiments the amphiphilic solvent is selected from ethyl acetate, ethyl lactate, propylene glycol, ethanol, glycerol, isopropanol, N-methylpyrrolidone, and liquid polyethylene glycol (PEG).

In numerous embodiments the formulation of the invention is constructed as a fast dissolution patch.

In certain embodiments the fast dissolution patch is configured to dissolve in a short dissolution time while releasing the PNS with the active agent into the buccal mucosa or into the oral cavity.

In further embodiments the dissolution time is between about 1 minute and about 8 hours.

In other embodiments the dissolution time is shorter than 30 minutes.

In numerous embodiments the fast dissolution patch comprising at least on film-forming compound.

In certain embodiments the film-forming compound is selected from sodium alginate, polyvinyl pyrrolidone, acrylic polymers, hydroxyl propyl cellulose and cellulose hydrophilic derivatives.

In yet another aspect the invention provides a buccal device for transmucosal delivery of a water-insoluble, hydrophobic or lipophilic drug agent to the bloodstream or to the oral cavity of a subject, the device comprising at least one PNS-containing depot configured for association or adherence to a tissue region of the subject’s oral cavity and for permitting contact with the tissue region, wherein the PNS is the formulation described above.

In another aspect the invention provides a buccal device for transmucosal systemic delivery of a water-insoluble, hydrophobic or lipophilic drug agent to a subject, the device being in the form of an enclosure comprising a PNS-containing depot, the enclosure having a tissue facing end configured for direct (intimate) contact between the PNS in the depot with a mucosal tissue of the subject, wherein the PNS is the formulation described above.

In yet another aspect the invention provides a buccal device configured for systemic delivery of a water-insoluble, hydrophobic or lipophilic drug agent to a subject, the device comprising a PNS-containing depot configured for release of the PNS transmucosally over a predetermined period of time, wherein the PNS is the formulation described above.

In numerous embodiments the buccal devices as above are configured to adhere to a mucosal tissue in a subject’s oral cavity while maintaining intimate contact with the tissue, the device allowing efficient transfer of the active agent present in the PNS to and through the mucosal tissue to the bloodstream or to the oral cavity.

In numerous embodiments the buccal devices as above are characterized in the PNS-containing depot comprises PNS or PNS co-mixed in a medium.

In further embodiments the medium is selected from hydrogel, oleogel and emulsion.

In certain embodiments the medium is a hydrogel.

In numerous embodiments the hydrogel is an edible gel.

In numerous embodiments the hydrogel comprises a gelling polymer.

In certain embodiments the gelling polymer is selected from gelatin, alginate, natural gum, hydroxypropyl cellulose, hydroxypropyl mehylcellulose and hyaluronic acid.

In numerous embodiments the devices of the invention are in a form having at least one exposed surface that can be intimately adhered or associated with the buccal tissue.

In certain embodiments the devices are in a form of a mucoadhesive patch or a tablet.

In further embodiments the shape of the mucoadhesive patch or tablet is selected from circular shape, oval shape and rectangular shape.

In numerous embodiments the size of the exposed area is between about 1 and about 4 cm².

In numerous embodiments the buccal devices of the invention are characterized in that the PNS-containing depot is incorporated into a reservoir or reservoirs, each comprising a reservoir base.

In certain embodiments the reservoir base comprises polyacrylic acid and a gelling agent.

In certain embodiments the polyacrylic acid is a crosslinked polyacrylic acid.

In further embodiments the crosslinked polyacrylic acid is carbopol or carbomer.

In certain embodiments the gelling agent is selected from acacia, alginic acid, bentonite, carboxymethyl cellulose, ethylcellulose, gelatin, hydroxyethyl cellulose, hydroxypropyl cellulose (HPS), magnesium aluminum silicate, methylcellulose, poloxamers, polyvinyl alcohol, sodium alginate, tragacanth, xanthan gum, hydroxypropyl-methyl-callulose (HPMC), hydroxypropyl starch calcium alginate and sodium alginate.

The other embodiments the gelling agent is selected from hydroxypropyl cellulose (HPC), hydroxypropyl-methyl-callulose (HPMC), hydroxypropyl starch, calcium alginate and sodium alginate.

In numerous embodiments the reservoir base which is in contact with the PNS-containing depot is a smooth surface.

In certain embodiments the reservoir base contains pores or cavities.

In certain embodiments the PNS-containing depot is incorporated into the smooth surface of the reservoir base.

In certain embodiments the PNS-containing depot is incorporated into the pores or cavities of the reservoir base.

In certain embodiments the inner surface of the reservoir base is coated with hydrophobic layer.

In further embodiments the hydrophobic layer is selected from wax and ethyl cellulose.

In numerous embodiments the reservoir base and the PNS-containing depot are covered with a protective layer, configured to be removed prior to application.

The numerous embodiments the crosslinked polyacrylic acid is carbopol.

The certain embodiments the amount of carbopol is between about 50% w/w and about 95% w/w.

The numerous embodiments the buccal devices of the invention further comprise at least one alcohol.

The certain embodiments the alcohol is selected from ethanol, propylene glycol, glycerol and short polyethylene glycol.

The further embodiments the alcohol is glycerol.

The certain embodiments the alcohol is in an amount ranging from about 5% to about 25% w/w.

The other embodiments the glycerol is at an amount of about 10% w/w.

The certain embodiments the reservoir base further comprising at least one other polymeric component.

The certain embodiments the polymeric component is selected from linear or crosslinked polyvinyl pyrrolidone, carrageenan, pullulan, and natural gum.

It is another objective of the invention to provide a method of transmucosal delivery of at least one water-insoluble active agent to a subject, the method comprising applying to the outermost tissue of the oral cavity one of the devices as described above, thereby enabling permeation of the active agent to the bloodstream or to the oral cavity of the subject.

From yet another aspect the invention provides a method of treating a disease or disorder, the method comprising applying to the outermost tissue of the oral cavity of a subject the devices as described above, wherein the device enabling delivery of the at least one active agent to a site of action.

In another aspect the invention provides a method of treating a disease treatable by apomorphine, the method comprising applying to the outermost tissue of the oral cavity of a subject the devices as described above, the device comprising apomorphine as the active agent, and wherein the apomorphine is in an amount effective to treat a disease treatable by apomorphine.

In still another aspect the invention provides a method of treating a disease treatable by at least one cannabinoid, the method comprising applying to the outermost tissue of the oral cavity of the devices as described above, the device comprising at least one cannabinoid as the active agent, and wherein the at least one cannabinoid is in an amount effective to treat the disease treatable by the at least one cannabinoid.

### Relevant Definitions

The ‘PNS-containing depot’ refers to the PNS formulation of the invention when used as is in a device of the invention, wherein the PNS is contained or held in a compartment in the device, or where the PNS is contained or co-mixed or incorporated in a medium or a material (which may be a single material or a composition of materials) in a form of a hydrogel, an oleogel or an emulsion. The emulsion may be an oil in water (o/w) emulsion or a water in oil (w/o) emulsion. Where the PNS is incorporated or dispersed in a hydrophilic material, e.g., a gel or a hydrogel, the resulting combination is in a form of a solid or semisolid material. The hydrophilic material is generally an edible material that comprises a gelling polymer such as gelatin, alginate, natural gum, hydroxypropyl cellulose, hydroxypropyl mehylcellulose and hyaluronic acid.

The depot may be contained in a chamber or enclosure within the buccal device or maybe the device itself.

In some embodiments, the PNS-containing depot is incorporated into a reservoir or reservoirs, each comprising a reservoir base. The base aims at preventing leakage of the PNS into the oral cavity. The base also maintains the PNS-containing depot in continuous contact with the buccal tissue thus enabling a prolonged and effective absorption of the active agent into said tissue and then to the bloodstream. Continuous contact is achievable by adhesion of the device to buccal tissue. The reservoir base is usually composed of a mixture of polyacrylic acid and a gelling agent. In some embodiments, the polyacrylic acid is a crosslinked polyacrylic acid. In some embodiments, the crosslinked polyacrylic acid may be carbopol and/or carbomer.

Thus, in certain aspects the invention provides a device for transmucosal delivery of a water-insoluble, hydrophobic or lipophilic drug agent to the bloodstream or to the oral cavity of a subject, the device comprising at least one PNS-containing depot configured for association or adherence to a tissue region of the subject’s oral cavity and for permitting contact with the tissue region.

The invention further provides a device for transmucosal systemic delivery of a water-insoluble, hydrophobic or lipophilic drug agent to a subject, the device being in the form of an enclosure comprising a PNS-containing depot, the enclosure having a tissue facing end configured for direct (intimate) contact between the PNS in the depot with a mucosal tissue of the subject.

A further device is provided, said device being a buccal device configured for systemic delivery of a water-insoluble, hydrophobic or lipophilic drug agent to a subject, the device comprising a PNS-containing depot configured for release of the PNS transmucosally over a predetermined period of time.

The ‘gelling agent’ or ‘gelling polymer’ is a thickening agent capable of increasing the viscosity of the base or the PNS without substantially changing its other properties. In accordance with the invention, the “gelling agent” is a component of the reservoir base, and the “gelling polymer” is a component of PNS-containing depot. Despite their different uses, both may be selected independently as indicated below.

The gelling agent may form a gel, or in other words, it may be dispersed in the liquid phase as a colloid mixture which forms an internal structure which is held in place by weak cohesive forces.

Non-limiting examples of such gelling agents may include acacia, alginic acid, bentonite, carboxymethyl cellulose, ethylcellulose, gelatin, hydroxyethyl cellulose, hydroxypropyl cellulose (HPS), magnesium aluminum silicate, methylcellulose, poloxamers, polyvinyl alcohol, sodium alginate, tragacanth, xanthan gum, hydroxypropyl-methyl-callulose (HPMC), hydroxypropyl starch and calcium/sodium alginate.

In some embodiments, the gelling agent may comprise hydroxypropyl cellulose (HPC), hydroxypropyl-methyl-callulose (HPMC), hydroxypropyl starch and calcium/sodium alginate.

The gelling polymers may be selected among such polymers indicated above.

In some embodiments, the surface of the reservoir base which is in contact with the PNS-containing depot is a smooth surface. In some embodiments, cavities or pores may be formed in the reservoir base. The PNS containing depot may be incorporated into the cavities which are formed on the reservoir base, or it may also be incorporated into the smooth surface of the reservoir base. To avoid desorption of water from the PNS containing depot to the reservoir base, a hydrophobic layer can be utilized for that purpose. Thus, the cavities of the reservoir base or the smooth surface of the reservoir base may be coated with a hydrophobic layer. In some embodiments of the disclosure, the hydrophobic layer can be selected from wax and ethyl cellulose.

Since the device is open in one of its ends to enable the release of the active agent contained in the PNS, the open end is prone to obstruction and contamination. To prevent such obstruction or contamination, the reservoir base part and the PNS-containing depot can be provided with a protective layer which can be removed prior to application.

Some of the reservoir base properties such as adhesion strength and flexibility may be controllable by altering the amounts or concentration of the materials which the base is made of, or by adding other materials. Adhesion strength can be determined by the amount of the crosslinked polyacrylic acid that is added when the reservoir base is prepared.

In some embodiments, the crosslinked polyacrylic acid is selected from carbomer and carbopol. In some embodiments, the crosslinked polyacrylic acid is carbopol.

In some embodiments, the content of the carbopol is between about 50% w/w to about 95% w/w of the total weight of the buccal device. In some embodiments, the content of the carbopol is between about 55% w/w to about 90% w/w. In some embodiments, the content of the carbopol is between about 60% w/w to about 90% w/w. In some embodiments, the content of the carbopol is between about 65% w/w to about 90% w/w. In some embodiments, the content of the carbopol is between about 70% w/w to about 90% w/w.

The flexibility of the device may also be altered, for example, by the addition of at least one alcohol. These alcohols may be selected from ethanol, propylene glycol, glycerol and short polyethylene glycol. In some embodiments, the alcohol is glycerol. In some other embodiments, the glycerol is present in an amount ranging from 5% to 25% w/w. In some embodiments, the glycerol is at an amount of about 10% w/w.

Other polymeric components can also be incorporated into the reservoir base. In some embodiments, such polymeric components are or comprise linear or crosslinked polyvinyl pyrrolidone, carrageenan, pullulan, and natural gum.

The PNS-containing depot may also be configured or constructed as a fast-dissolving patch, which upon contact with the buccal mucosa tissue, dissolves in a short dissolution time while releasing the PNS with the active agent into the buccal mucosa or into the oral cavity. In some embodiments, the dissolution time is between about 1 min to about 8 h. In some other embodiments, the dissolution time is shorter than 30 min.

To obtain a fast dissolution profile, a film-forming compound may be utilized. The ‘film-forming compound’ may be any compound which upon contact with an aqueous solution forms a gel with strong hydrophilic properties. Such a gel exhibits fast dissolution features as disclosed herein. Examples of a film-forming compound may include, but are not limited to, sodium alginate, polyvinyl pyrrolidone, acrylic polymers, hydroxyl propyl cellulose and cellulose hydrophilic derivatives.

1-79. (canceled)

80. A solvent-free device for controlled delivery of at least one poorly water-soluble or lipophilic active through at least one tissue of the oral cavity of a subject, the device being a solid fast dissolving device comprising at least one water-soluble polymer and at least one edible alcohol; and at least one atomized poorly water-soluble or lipophilic active comprised in a solid pre-formulation with at least one dispersant, at least one lipid component and at least one amphiphilic material.

81. The device of claim 80, wherein the device adheres to at least one tissue of the oral cavity, or wherein the device is in a form of a tablet or a film compatible with the size of the oral cavity.

82. The device of claim 80 having a controlled adherence or adhesiveness to at least one tissue or organ of the oral cavity over a predetermined period of time.

83. The device of claim 80, wherein said at least one poorly water-insoluble or lipophilic active is selected from the group of analgesics, anesthetics, antiseptics, antibacterial agents, antiviral agents, disinfectants, herbal extracts, anti-halitosis agents, anti-inflammatory agents, opioids, cardiovascular drugs, caffeine, nicotine, mood stabilizing or stimulating agents and antidepressants.

84. The device of claim 80, wherein said at least one poorly water-insoluble or lipophilic active is a biological active selected from the group of peptides, proteins, enzymes, and single- or double-stranded nucleic acids.

85. The device of claim 80, wherein said at least one poorly water-insoluble or lipophilic active is selected from the group of small and peptide drugs, herbal and animal extracts, homeopathic agents, nutraceuticals, vitamins, probiotic bacteria, and combination thereof.

86. The device of claim 80, wherein said at least one poorly water-insoluble or lipophilic active is selected from the group of cannabinoids, curcumin, apomorphine, amphotericin B, cyclosporine, and rapamycin.

87. The device of claim 86, wherein said at least one poorly water-insoluble or lipophilic active is at least one cannabinoid having affinity to cannabinoid receptor type 1 (CB1) or cannabinoid receptor type 2 (CB2).

88. The device of claim 87, wherein the at least one cannabinoid is selected from the group of cannabidiol (CBD), delta-9-tetrahydrocannabinol (THC), cannabichromene (CBC), cannabichromenic acid (CBCA), cannabichromevarin (CBCV), cannabichromevarinic acid (CBCVA), cannabicyclol (CBL), cannabicyclolic acid (CBLA), cannabicyclovarin (CBLV), cannabidiol monomethylether (CBDM), cannabidiolic acid (CBDA), cannabidiorcol (CBD-C1), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), cannabinodiol (CBND), cannabinodivarin (CBVD), cannabinol (CBN), cannabinol methylether (CBNM), cannabivarin (CBV), cannabitriol (CBT), cannabitriolvarin (CBTV), delta-8-tetrahydrocannabinol (Δ8-THC), delta-8-tetrahydrocannabinolic acid (Δ8-THCA), delta-9-tetrahydrocannabivarin (THCV), delta-9-tetrahydrocannabivarinic acid (THCVA) and cannabicitran (CBT) or any combination thereof.

89. The device of claim 87, wherein the at least one cannabinoid is comprised in an extract of a cannabis plant or any part thereof.

90. The device of claim 87, wherein the at least one cannabinoid is THC and/or CBD.

91. The device of claim 80, wherein said at least one poorly water-soluble or lipophilic active comprised in the pre-formulation is atomized to a particle size in the range between about 1 nm and about 1000 nm or between 20 nm and 500 nm.

92. A solid fast dissolving dosage form for a controlled delivery of at least one poorly water-soluble or lipophilic active through at least one tissue in the oral cavity, comprising at least one poly(carboxylic acid) and at least one edible alcohol and the at least one poorly water-soluble or lipophilic active comprised in a pre-formulation with at least one dispersant, at least one lipid component and at least one amphiphilic solvent wherein said active is atomized,
the dosage form and the pre-formulation of the poorly water-soluble or lipophilic active(s) are solid and solvent free, and
the dosage form has a preferential solubility in the oral cavity, whereby the pre-formulation of the poorly water-soluble or lipophilic active(s) is released and delivered through the at least one tissue of the oral cavity.

93. A device of a claim 80 for use in treating at least one disorder, or at least one pre-clinical or clinical condition treatable by one or more cannabinoid(s).

94. A solvent-free device for prolonged delivery of at least one water-soluble or hydrophilic active through at least one tissue of the oral cavity of a subject, the device being a solid fast dissolving device comprising at least one water-soluble polymer and at least one edible alcohol and at least one water-soluble active.
